(19) United States
(12) Patent Application Publication
(10) Pub. No.: US 2025/0354156 A1
(43) Pub. Date: Nov. 20, 2025

(54) Compositions and Methods for Treating Cancer

(71) Applicant: bioAffinity Technologies, Inc., San Antonio, TX (US)

(72) Inventors: David J. Elzi, San Antonio, TX (US); William E. Bauta, San Antonio, TX (US); Vivienne I. Rebel, San Antonio, TX (US)

(73) Assignee: bioAffinity Technologies, Inc., San Antonio, TX (US)

(21) Appl. No.: 19/212,343

(22) Filed: May 19, 2025

Related U.S. Application Data
(63) Continuation of application No. 17/359,905, filed on Jun. 28, 2021, now Pat. No. 12,305,171.
Application No. 17/359,905 is a continuation-in-part of application No. PCT/US2019/068423, filed on Dec. 23, 2019.
(60) Provisional application No. 62/785,592, filed on Dec. 27, 2018.
(60) Provisional application No. 63/044,771, filed on Jun. 26, 2020.

(51) Int. Cl. C12N 15/113

(52) U.S. Cl. CPC C12N 15/1138; C12N 2310/14; C12N 2310/315; C12N 2320/30

(57) ABSTRACT

A method of inhibiting skin cancer by administering to a subject in need thereof a double stranded RNA interference (RNAi) agent comprising at least one of (i) a first double-stranded ribonucleic acid (dsRNA) for inhibiting the expression of a CD320 gene wherein the first dsRNA comprises a sense strand and an antisense strand forming a duplex, and (ii) a second dsRNA for inhibiting the expression of a LRP2 gene wherein the second dsRNA comprises a sense strand and an antisense strand forming a duplex, wherein the sense strand of the first dsRNA is at least substantially complementary to the antisense strand of the first dsRNA and the sense strand of the second dsRNA is at least substantially complementary to the antisense strand of the second dsRNA and the use of the RNAi agent as a pharmaceutical composition for the treatment of cancer in subjects in need of treatment.

### CROSS-REFERENCE TO RELATED APPLICATIONS

This application is a continuation application of U.S. patent application Ser. No. 17/359,905, filed on Jun. 28, 2021, titled “Compositions and Methods for Treating Cancer”, which is a Continuation-In-Part application of International Patent Application No. PCT/US2019/068423, filed on Dec. 23, 2019, titled “Compositions and Methods for Treating Cancer”, which claim priority to and the benefit of U.S. Provisional Patent Application No. 62/785,592, titled “Compositions and Methods for Treating Cancer”, filed on Dec. 27, 2018. U.S. patent application Ser. No. 17/359,905 also claims priority to and the benefit of the filing of U.S. Provisional Patent Application No. 63/044,771, filed on Jun. 26, 2020, titled “Compositions and Methods for Treating Cancer”. The specification and claims thereof are incorporated herein by reference.

### SEQUENCE LISTING

The instant application contains a Sequence Listing which has been submitted electronically in XML format and is hereby incorporated by reference in its entirety. Said XML copy, created on Jul. 1, 2025, is named Replacement_Compositions and Methods for Treating Cancer_Con.xml and is 2.12 MB in size.

### BACKGROUND

A variety of cancer therapies and treatments exist such as surgical resection of solid tumors, radiation, and chemotherapy. While surgical resection and radiation are used on localized tumors, chemotherapy is often delivered systemically and impacts both cancer and non-cancer cells, leading to severe and even life-threatening side effects. Older cancer drugs, including alkylators, nucleotide antimetabolites, and tubulin poisons, cause significant side effects because they are similarly toxic to normal cells as to cancer cells, especially those normal cells undergoing routine cell division in the intestine, scalp, and skin. For this reason, much of the effort in contemporary cancer drug discovery is devoted to finding targeted therapeutics which differentiate between cancer cells and normal cells (Neidle et al., (2014) Cancer Drug Design and Discovery). This has led to drugs which inhibit the function of oncolytic proteins that are mutated, overexpressed, or abnormally hyperactive in cancer but not in normal cells. Examples of such drugs include kinase inhibitors, histone deacetylase inhibitors, proteasome inhibitors, mTOR inhibitors, BCL2 inhibitors, and isocitrate dehydrogenase inhibitors. Significant effort has also been devoted to targeting cell surface antigens which are differentially expressed in cancer cells compared to normal cells. Monoclonal antibodies and antibody-drug conjugates targeting cancer cell surface antigens have thus been developed as cancer therapeutics (Beck et al., (2017) Nat Rev Drug Disc 16, 315-337). Another point of differentiation between cancer cells and normal cells is metabolism. It was discovered many years ago that many cancer cells utilize glucose fermentation to generate ATP as opposed to the process of oxidative phosphorylation used by normal cells. A drug targeting isocitrate dehydrogenase, involved in abnormal glucose metabolism in cancer cells, was recently approved by the FDA (Dhillon (2018) Drugs 78, 1509-1516). Abnormalities in one-carbon metabolism, which encompasses the folate and methionine cycles and affects nucleotide synthesis and DNA methylation as a way of controlling gene expression, are strongly associated with some cancers (Fanidi et al., (2019) Int J Cancer 145, 1499-1503; Yang (2018) Front Oncol 8, 493). In this connection, it has been known for a long time that certain synthetic analogs of folic acid (antifolates) can inhibit the growth of cancer cells. It is also known that some cancer cells are dependent for survival on the amino acid methionine. If methionine is restricted, the cancer cells die, while this has little effect on normal cells. In recent years, evidence has begun to emerge that some cancer cells might have an abnormal dependency on vitamin B12. The nature of this dependency is not understood but might, in part, involve the use of vitamin B12 as a catalytic cofactor by the enzyme methionine synthase in one-carbon metabolism.

Vitamin B12 (cobalamin) is an essential micronutrient in the human diet. It is a cofactor for the metabolic enzymes methionine synthase and methylmalonyl-CoA mutase (Fedosov et al., (2012) Water Soluble Vitamins (book) 56, 347-367). After oral ingestion and transport through the intestine, cobalamin is almost completely protein bound in plasma to the chaperone proteins transcobalamin 1 (TCN1, haptocorrin, R-binder) (TCO1_HUMAN) and transcobalamin 2 (TCN2) (TCO2_HUMAN). The TCN2-cobalamin complex (TCN2-Cbl) is taken up by most cells using the process of receptor-mediated endocytosis and has a plasma half-life of 1-15 h. TCN2 has a high affinity and specificity for cobalamin in its various dietary and nutritional supplement forms, such as methyl cobalamin, adenosyl cobalamin and cyanocobalamin (Fedosov et al., (2007) Biochem 46, 6446-6458). TCN1 is a glycoprotein that exists in two different forms in plasma (Marzolo and Farfan (2011) Biol Res 44, 81-105). The most abundant form is sialylated and has a plasma half-life of about 10 days (Bor (2004) Clin Chem 50, 1043-1049). A less abundant form is desialylated and has a plasma half-life of a few minutes. Unlike TCN2-Cbl, which can be taken up by almost all cell types, the transcobalamin 1-cobalamin complex (TCN1-Cbl) is quickly taken up by certain liver cells, only in its desialylated form, by receptor-mediated endocytosis.

CD320 and LRP2 are two receptors involved in the uptake of cobalamin as TCN2-Cbl. CD320, a member of the low-density lipoprotein receptor (LDLR) family, is constitutively expressed in most cells and is the receptor primarily responsible for the uptake of cobalamin (Quadros (2013) Biochimie 95, 1008-1018). CD320 is overexpressed in some types of cancer (Sycel et al., (2013) Anticancer Res 33, 4203-4212; Amagasaki (1990) Blood 76, 1380-1386). There is also evidence that CD320 facilitates the transport of TCN2-Cbl through the blood-brain barrier into the brain (Lai et al.; (2013) FASEB 27, 2468-2475). LRP2 is another receptor in the LDLR family. It is expressed most highly in the kidney but also in other tissues. In addition to cobalamin, LRP2 also transports sundry proteins and small molecules, including albumin, insulin and vitamin D (Mazolo et al., (2011) Biol Res 44, 89-105). In the liver, the asialoglycoprotein receptor (ASGR) uptakes TCN1-Cbl by receptor-mediated endocytosis so long as TCN1 is in its desialylated form. Normal liver cells and liver cancer cells express very high levels of ASGR (˜50,000 receptors per cell), making this receptor attractive as a portal for delivering drugs to the liver (Luo et al., (2017) Biomedicine and Pharmacotherapy 88, 87-94; Stockert (1995) Physiological Rev 75, 595-609; Soda et al., Blood (1985) 65, 795-802).

After receptor mediated endocytosis, cobalamin is sequestered in the endosome, where the endosomal membrane prevents passive egress to the cytosol. A specialized protein (cbIF) facilitates the transport of cobalamin through the endosomal membrane to the cytosol (Banerjee et al., (2009) Curr Opin Chem Bio 13, 484-491).

### BRIEF SUMMARY OF THE INVENTION

One embodiment of the present invention provides for a double stranded RNA interference (RNAi) agent comprising at least one of (i) a first double-stranded ribonucleic acid (dsRNA) for inhibiting the expression of a CD320 gene wherein the first dsRNA comprises a sense strand and an antisense strand forming a duplex, (ii) a second dsRNA for inhibiting the expression of a LRP2 gene wherein the second dsRNA comprises a sense strand and an antisense strand forming a duplex, or (iii) a cocktail of (i) and (ii) and wherein the sense strand of the first dsRNA is at least substantially complementary to the antisense strand of the first dsRNA and the sense strand of the second dsRNA is at least substantially complementary to the antisense strand of the second dsRNA. For example, the antisense strand of (i) the first dsRNA includes a region of complementarity to a CD320 RNA transcript and for example the sense strand of (i) the first dsRNA is selected from Table 5. The antisense strand of (ii) the second dsRNA includes a region of complementarity to an LRP2 RNA transcript and the sense strand of (ii) the second dsRNA are selected from Table 6. In one example, (i) the first dsRNA or (ii) the second dsRNA comprises a duplex region which is 16-30 nucleotide pairs in length. In another example, (i) the first dsRNA or (ii) the second dsRNA comprises a duplex region which is 21-23 nucleotide pairs in length. In one embodiment, the double stranded RNAi agent includes at least one strand of: (i) the first dsRNA or (ii) the second dsRNA which comprises a 3′ overhang of at least 2 nucleotides. Further still, in one embodiment, the antisense strand of (i) the first dsRNA, comprises the nucleotide sequence selected from (5′→3′):

```
  (SEQ ID NO: 17)
CAGUUGCGCAGUUUCUUGUCAGUUCdTdT;
  (SEQ ID NO 18)
CAGUUGCGCAGUUUCUUGUCAGUUCdT*dT;
  (SEQ ID NO 19)
mCmAmGmUmUmGmCmGmCmAmGmUmUmUmCmUmUmGmUmCmAmGmUmUmCdT*dT;
  (SEQ ID NO 21)
mCmAmGmUmUmGmCmGmCmAmGmUmUmUmCmUmUmGmUmCmAmGmUmUmC;
  (SEQ ID NO 23)
mCmAmGmUmUmGmCmGmCmAmGmUmUmUmCmUmUmGmUmCmAmGmUmUmCdT*dT;
  (SEQ ID NO 24)
mC2fAmG2fUmU2fGmC2fGmC2fAmG2fUmU2fUmC2fUmU2fGmU2fCmA2fGmU2fUmCdT*dT;
  (SEQ ID NO 25)
mC2fAmG2fUmU2fGmC2fGmC2fAmG2fUmU2fUmC2fUmU2fGmU2fCmA2fGmU2fUmC;
  (SEQ ID NO 28)
2fCmA2fGmU2fUmG2fCmG2fCmA2fGmU2fUmU2fCmU2fUmG2fUmC2fAmG2fUmU2fCdT*dT;
  (SEQ ID NO 29)
2fCmA2fGmU2fUmG2fCmG2fCmA2fGmU2fUmU2fCmU2fUmG2fUmC2fAmG2fUmU2fC;
  (SEQ ID NO 30)
mC2fA2fG2fU2fU2fG2fC2fG2fC2fA2fG2fU2fU2fU2fC2fU2fU2fG2fU2fC2fA2fG2fU2fU
2fCdT*dT;
  (SEQ ID NO 32)
mC2fAmG2fUmU2fGmC2fGmC2fAmG2fUmU2fUmC2fUmU2fGmU2fCmA2fGmU2fUmCdT*dT;
  (SEQ ID NO 33)
mC2fAmG2fUmU2fGmC2fGmC2fAmG2fUmU2fUmC2fUmU2fGmU2fCmA2fGmU;
  (SEQ ID NO 34)
mC2fAmG2fUmU2fGmC2fGmC2fAmG2fUmU2fUmC2fU2fU2fG2fU2fC2fA2fG2fU;
wherein, mA, mC, mG, and mU are 2′-O-methyl adenosine, cytidine,
guanosine, or uridine, respectively; 2fA, 2fC, 2fG, and 2fU are 2′-fluoro
adenosine, cytidine, guanosine, or uridine, respectively; and * is a
phosphorothioate linkage; and the sense strand is at least substantially
complementary to the antisense strand.
```

Further still, in another embodiment, the double stranded RNAi agent includes the antisense strand of (i) the first dsRNA, that comprises the nucleotide sequence selected from (5′→3′)

```
  (SEQ ID NO 64)
AAGAGCUCAGGUCUCUGAGGGdTdT;
  (SEQ ID NO 65)
AAGAGCUCAGGUCUCUGAGGGdT*dT;
  (SEQ ID NO 66)
mAmAmGmAmGmCmUmCmAmGmGmUmCmUmCmUmGmAmGmGmGdT*dT;
  (SEQ ID NO 68)
mAmAmGmAmGmCmUmCmAmGmGmUmCmUmCmUmGmAmGmGmG;
  (SEQ ID NO 71)
mA2fAmG2fAmG2fCmU2fCmA2fGmG2fUmC2fUmC2fUmG2fAmG2fGmGdT*dT;
  (SEQ ID NO 72)
mA2fAmG2fAmG2fCmU2fCmA2fGmG2fUmC2fUmC2fUmG2fAmG2fGmG;
  (SEQ ID NO 75)
2fAmA2fGmA2fGmC2fUmC2fAmG2fGmU2fCmU2fCmU2fGmA2fGmG2fGdT*dT;
  (SEQ ID NO 76)
2fAmA2fGmA2fGmC2fUmC2fAmG2fGmU2fCmU2fCmU2fGmA2fGmG2fG;
  (SEQ ID NO 77)
mA2fA2fGmA2fGmC2fUmC2fAmG2fGmU2fCmU2fCmU2fGmA2fGmG2fG;
  (SEQ ID NO 78)
mA2fA2fGmA2fGmC2fUmC2fAmG2fGmU2fCmU2fCmU2fGmA2fGmG2fGdT*dT;
  (SEQ ID NO 79)
2fAmA2fGmA2fGmC2fUmC2fAmG2fGmU2fCmU2fCmU2fGmA2fGmG2fGdT*dT;
  (SEQ ID NO 81)
2fAmA2fGmA2fGmC2fUmC2fAmG2fGmU2fCmU2fC2fU2fG2fA2fG2fG2fG;
wherein, mA, mC, mG, and mU are 2′-O-methyl adenosine, cytidine,
guanosine, or uridine, respectively; 2fA, 2fC, 2fG, and 2fU are
2′-fluoro adenosine, cytidine, guanosine, or uridine, respectively;
and * is a phosphorothioate linkage; and the sense strand is
at least substantially complementary to the antisense strand.
```

In another embodiment the double stranded RNAI agent of (i) the second deRNA comprises the nucleotide sequence selected from (5′→3)

```
  (SEQ ID NO: 417)
UUUGAUAGCACCAAACCUAGAGCCCdTdT;
  (SEQ ID NO: 418)
UUUGAUAGCACCAAACCUAGAGCCCdT*dT;
  (SEQ ID NO: 419)
mUm[mUmGmAmUmAmGmCmAmCmCmAmAmAmCmCmUmAmGmAmGmCmCmCdT*dT;
  (SEQ ID NO: 421)
mUmUmUmGmAmUmAmGmCmAmCmCmAmAmAmCmCmUmAmGmAmGmCmCmC;
(SEQ ID NO: 424)
mU2fUmU2fGmA2fUmA2fGmC2fAmC2fCmA2fAmA2fCmC2fUmA2fGmA2fGmC2fCmCdT*dT];
  (SEQ ID NO: 425)
mU2fUmU2fGmA2fUmA2fGmC2fAmC2fCmA2fAmA2fCmC2fUmA2fGmA2fGmC2fCmC;
  (SEQ ID NO: 429)
mU2fAmU2fCmA2fAmA2fCmC2fUmC2fGmA2fUmA2fGmC2fAmA2fCmA2fCmC2fGmC;
  (SEQ ID NO: 430)
mU2fU2fU2fG2fA2fU2fA2fG2fC2fA2fC2fC2fA2fA2fA2fC2fC2fU2fA2fG2fA2fG2fC2fC2fCdT*dT;
  (SEQ ID NO: 432)
mU2fUmU2fGmA2fUmA2fGmC2fAmC2fCmA2fAmA2fCmC2fUmA2fGmA2fGmC2fCmCdT*dT;
  (SEQ ID NO: 433)
mU2fUmU2fGmA2fUmA2fGmC2fAmC2fCmA2fAmA2fCmC2fUmA2fGmA2fGmC;
and
  (SEQ ID NO: 434)
mU2fUmU2fGmA2fUmA2fGmC2fAmC2fCmA2fAmA2fC2fC2fU2fA2fG2fA2fG2fC
wherein, mA, mC, mG, and mU are 2′-O-methyl adenosine, cytidine,
guanosine, or uridine, respectively; 2fA, 2fC, 2fG, and 2fU are
2′-fluoro adenosine, cytidine, guanosine, or uridine, respectively;
and * is a phosphorothioate linkage; and the sense strand is at
least substantially complementary to the antisense strand.
```

In a further embodiment, the double stranded RNAI agent antisense strand of (i) the second deRNA comprises the nucleotide sequence selected from (5′→3)

```
(SEQ ID NO: 448)
UUUGCAAUGACUCUCCUAUCAGUCCdTdT;
  (SEQ ID NO: 449)
UUUGCAAUGACUCUCCUAUCAGUCCdT*dT;
(SEQ ID NO: 450)
mUmUmUmGmCmAmAmUmGmAmCmUmCmUmCmCmUmAmUmCmAmGmUmCmCdT*dT;
(SEQ ID NO: 452)
mUmUmUmGmCmAmAmUmGmAmCmUmCmUmCmCmUmAmUmCmAmGmUmCmC;
(SEQ ID NO: 455)
mU2fUmU2fGmC2fAmA2fUmG2fAmC2fUmC2fUmC2fCmU2fAmU2fCmA2fGmU2fCmCdT*dT;
(SEQ ID NO: 456)
mU2fUmU2fGmC2fAmA2fUmG2fAmC2fUmC2fUmC2fCmU2fAmU2fCmA2fGmU2fCmC;
  (SEQ ID NO: 458)
mU2fUmU2fGmC2fAmA2fUmG2fAmC2fUmC2fUmC2fCmU2fAmU2fCmA2fCmU2fCmC;
(SEQ ID NO: 459)
2fUmU2fUmG2fCmA2fAmU2fGmA2fCmU2fCmU2fCmC2fUmA2fUmC2fAmG2fUmC2fCdT*dT;
(SEQ ID NO: 460)
mU2fAmU2fCmC2fUmA2fAmG2fUmC2fAmC2fAmC2fGmU2fUmU2fGmA2fCmU2fGmC;
(SEQ ID NO: 461)
mU2fU2fU2fG2fC2fA2fA2fU2fG2fA2fC2fU2fC2fU2fC2fC2fU2fA2fU2fC2fA2fG2fU2fC2fCdT*dT ;
  (SEQ ID NO: 463)
mU2fUmU2fGmC2fAmA2fUmG2fAmC2fUmC2fUmC2fCmU2fAmU2fCmA2fGmU2fCmCdT*dT;
  (SEQ ID NO: 464)
mU2fUmU2fGmC2fAmA2fUmG2fAmC2fUmC2fUmC2fCmU2fAmU2fCmA2fGmU;
  (SEQ ID NO: 465)
mU2fUmU2fGmC2fAmA2fUmG2fAmC2fUmC2fUmC2fC2fU2fA2fU2fC2fA2fG2fU
wherein, mA, mC, mG, and mU are 2′-O-methyl adenosine, cytidine, guanosine,
or uridine, respectively; 2fA, 2fC, 2fG, and 2fU are 2′-fluoro adenosine,
cytidine, guanosine, or uridine, respectively; and * is a phosphorothioate
linkage; and the sense strand is at least substantially complementary to the
antisense strand.
```

For example, when the RNAi agent comprises (iii) the combination of (i) the first dsRNA and (ii) the second dsRNA, the antisense strand of (i) the first dsRNA is selected from

(SEQ ID NO: 17)
CAGUUGCGCAGUUUCUUGUCAGUUCdTdT;
(SEQ ID NO 18)
CAGUUGCGCAGUUUCUUGUCAGUUCdT*dT
(SEQ ID NO 64)
AAGAGCUCAGGUCUCUGAGGGdTdT;
and
(SEQ ID NO 65)
AAGAGCUCAGGUCUCUGAGGGdT*dT;
and
the antisense strand of (ii) the second dsRNA is
selected from
(SEQ ID NO: 417)
UUUGAUAGCACCAAACCUAGAGCCCdTdT;
(SEQ ID NO: 418)
UUUGAUAGCACCAAACCUAGAGCCCdT*dT;
(SEQ ID NO: 448)
UUUGCAAUGACUCUCCUAUCAGUCCdTdT;
and
(SEQ ID NO: 449)
UUUGCAAUGACUCUCCUAUCAGUCCdT*dT;
wherein * is a phosphorothioate linkage; and
the sense strand is at least substantially
complementary to the antisense strand.

In one embodiment, (i) the first dsRNA has the duplex structure of (SEQ ID NOS: 17 and 110) or (SEQ ID NOs: 18 and 111). In another (ii) the second dsRNA has the duplex structure of (SEQ ID NOs: 417 and 808) or (SEQ ID NOs: 448 and 822).

Another embodiment provides for an isolated cell comprising a double stranded RNAi gent of (i), (ii) or (iii).

For example, the sense strand of (i) the first dsRNA is no more than 30 nucleotides in length, and the antisense strand of (i) the first dsRNA is no more than 30 nucleotides in length. For example, the sense strand of (ii) the second dsRNA is no more than 30 nucleotides in length, and the antisense strand is no more than 30 nucleotides in length.

Yet another embodiment provides a pharmaceutical composition for inhibiting expression of a CD320 gene, the pharmaceutical composition comprising a double stranded RNAi agent (i) or (iii). Further the pharmaceutical composition may include an excipient.

Yet another embodiment provides a pharmaceutical composition for inhibiting expression of an LRP2 gene, the composition comprising a double stranded RNAi agent (ii) or (iii). Further the pharmaceutical composition may include an excipient.

Another embodiment of the present invention provides a method for inhibiting proliferation of a cancer cell (CC) comprising contacting of the CC with an inhibitor of CD320 add/or LRP2 in an amount effective to inhibit proliferation of the CC. For example, the CC may express CD320 and/or LRP2 or both.

Another embodiment of the present invention provides a method for treating a therapeutically-resistant cancer in a subject who has previously received a therapy, comprising administering to the subject an inhibitor of CD320 add/or LRP2 in an amount effective to inhibit or kill cancer cells (CCs) present in the therapeutically-resistant cancer.

Another embodiment of the present invention provides a method for treating cancer in a subject who has recurring or relapsed cancer comprising administering to a subject an inhibitor of CD320 add/or LRP2 in an amount effective to inhibit or kill CCs in the cancer.

The CC is from a cancer selected from melanoma, glioblastoma, lung carcinoma, breast carcinoma, triple negative breast carcinoma, hepatocellular carcinoma, renal carcinoma, pancreatic carcinoma, ovarian carcinoma and prostate carcinoma.

The CD320 inhibitor is selected from an antibody that binds CD320, a small molecule inhibitor of CD320, and a RNAi agent that hybridizes to a nucleic acid sequence encoding CD320.

Further, the method of inhibiting proliferation of a CC, treating a therapeutically resistive cancer in a subject or has a recurring or relapsed cancer comprises administering a cancer therapeutic in combination with an RNAi agent that hybridizes to an mRNA encoding for CD320 or an RNAi agent that hybridizes to an mRNA encoding for LRP2. For example, the cancer therapeutic is selected from the antifolate class, epigenetic modulatory class, or a small molecule or protein inhibitor of CD320 function or LRP2 function, such as an antibody for CD320 or an antibody for LRP2. Further still, the method further comprises administering metformin. For example, the RNAi agent comprises an antisense strand of Table 5 or of Table 6.

The inhibitor is selected from the group consisting of an antibody that binds LRP2, a small molecule inhibitor of LRP2, and an RNAi agent that hybridizes to a nucleic acid sequence encoding LRP2. For example, the method further comprises administering a cancer therapeutic selected from the antifolate class, epigenetic modulatory class, or the small molecule or protein inhibitor of LRP2 function, such as an antibody, in combination with an RNAi agent that hybridizes to an mRNA encoding for LRP2.

The method further comprises administering a cancer therapeutic in combination with an RNAi agent that hybridizes to an mRNA encoding for LRP2.

One embodiment of the present invention provides for a method for inhibiting proliferation of a cancer cell (CC) comprising contacting of a CC with a composition comprising an inhibitor of CD320 and an inhibitor of LRP2 in an amount effective to inhibit proliferation of the CC. For example, the composition is a cocktail comprising i) the CD320 inhibitor selected from an antibody that binds CD320, a small molecule inhibitor of CD320, and a RNAi agent that hybridizes to a nucleic acid encoding CD320 and any combination thereof, and ii) the LRP2 inhibitor selected from an antibody that binds LRP2, a small molecule inhibitor of LRP2, and a RNAi agent that hybridizes to a nucleic acid sequence encoding LRP2 and any combination thereof. Further, the method further comprises administering a cancer therapeutic selected from the antifolate class and epigenetic modulatory class. For example, the RNAi agent that hybridizes to the mRNA encoding for CD320 comprises a first double-stranded ribonucleic acid (dsRNA) for inhibiting expression of CD320, wherein the first dsRNA comprises a sense strand and an antisense strand, the antisense strand comprising a region of complementarity to a CD320 RNA transcript and the RNAi agent that hybridizes to the mRNA encoding for LRP2 comprises a second dsRNA for inhibiting expression of LRP2, wherein the second dsRNA comprises a sense strand and an antisense strand, the antisense strand comprising a region of complementarity to an LRP2 RNA transcript. In a further example, the antisense strand that is complementary to CD320 RNA transcript is selected from Table 5 and the antisense strand that is complementary to the RNA transcript for LRP2 is selected from Table 6. The method further comprises administering a cancer therapeutic selected from the antifolate class and epigenetic modulatory class. The method further comprises administering a cancer therapeutic selected from the immunomodulatory class. Further still, the method further comprises administering metformin.

One aspect of one embodiment of the present invention provides a method for the inhibition of CD320 and LRP2 protein expression, such that the levels of these proteins are reduced in treated cells compared to their endogenous levels in untreated cells; this inhibition may also be referred to as the knockdown of CD320 and LRP2 expression. The method entails the use of a cocktail of small interfering RNA molecules, otherwise known as siRNAs, which guide the mRNA sequences encoding for either CD320 or LRP2 into an enzymatic complex which leads to targeted destruction of these mRNAs.

Another aspect of the present invention provides a method for the individual or concurrent inhibition of LRP2 and CD320 protein expression, which inhibits the growth of many cancer cells as compared to non-cancer (normal) cells. In some instances, CD320 or LRP2 protein knockdown alone is sufficient to severely inhibit cancer cell proliferation compared to normal cells.

Another aspect of the present invention provides for inhibition of cancer cell proliferation by inhibiting LRP2 receptor expression.

Mechanistic investigations into the selectivity of porphyrin uptake by cancer cells led to several nonobvious compounds and methods of using the compound(s). It was discovered that the knockdown of the expression of either CD320 gene or LRP2 gene or the simultaneous knockdown of the expression of CD320 gene and LRP2 gene caused cell death or inhibition of cell growth in a panel of lung cancer cell lines, compared to normal fibroblasts. The experimental outline is illustrated in FIG. 1. In these experiments, cells were plated on day 0. The next day (day 1), virus particles encoding short hairpin RNAs (shRNAs) directed to the CD320 gene and the LRP2 gene or an irrelevant shRNA control were added to the cell culture together with protamine sulfate, a reagent that facilitates cell entry of the virus particles.

Further investigations revealed that knockdown of the expression of either the CD320 gene or LRP2 gene or the simultaneous knockdown of the expression of CD320 and LRP2 genes using small interfering RNAs (siRNAs) caused cell death or inhibition of cell growth in a panel of cancer cell lines that included lung cancer, prostate cancer, breast cancer, glioblastoma and melanoma, compared to normal fibroblasts (FIG. 9-10). It was also found that that knockdown of one gene, either CD320 or LRP2, led to increased expression of the other in some cancer cell lines.

One aspect of the present invention provides for the knockdown of the CD320 receptor, the LRP2 receptor or the simultaneous knockdown of both in vivo and in vitro cancer cells that express CD320 mRNA and/or LRP2 mRNA.

Another aspect of the present invention is a method to inhibit cell growth or cause cell death of cancer cells treated with a compound as described herein, while leaving normal cells unaffected or inhibiting cell growth to a lesser degree or producing less cell death as compared to a cancer cell treated with the same amount of the compound.

Another aspect of a first compound and method of use is a selective therapy which inhibits proliferation of cancer cells and/or kills cancer cells with an inhibition of LRP2 Receptor while leaving normal cells unharmed.

Another aspect of a second compound and method of use is a selective therapy which inhibits proliferation of cancer cells and/or kills cancer cells with an inhibition of CD320 Receptor while leaving normal cells unharmed.

Another aspect of the present invention provides for treating a cancer by administering a therapy to selectively inhibit proliferation of a cancer cell(s) and/or kill a cancer cell(s) with one or more of the following, a first compound that is an inhibitor of CD320 receptor, a second compound that is an inhibitor of LRP2 receptor or a combination thereof.

### DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 illustrates an experimental design for knocking down CD320 and LRP2 in a cell. Cells were plated on day 0. The next day (day 1), virus particles encoding short hairpin RNAs (shRNAs) directed at the CD320 and LRP2 mRNA or a non-targeting shRNA control were added to the cell culture together with protamine sulfate, a reagent that facilitates cell entry of the virus particles. Table 1 shows the sequences that were used. Each shRNA coding sequence was also combined with a unique drug resistance gene, which would allow for selecting those cells that had taken up the shRNA; cells that had not taken up the shRNA would not survive. On day 2, drug selection was started. On day 3, the cells were harvested and plated in a new dish. Only the cells with a drug resistance gene, i.e., those cells that had taken up shRNA virus particles would survive this re-plating procedure. From day 4 on, each culture was closely observed for cell growth. Cells infected with the non-targeting negative control shRNA continued growing-data not shown. The results for the cell lines that expressed the CD320+LRP2 shRNAs are shown in Table 1.

FIGS. 2A-C illustrate sensitivity of cancer cell lines to knockdown of CD320 and LRP2. Normal cells (GM05659 fibroblasts) or cancer cells were infected with lentiviruses expressing shRNAs to control sequences or to shCD320 and shLRP2 as described in FIG. 1. The cells were grown as described in FIG. 1. On the ninth day after transfection with the lentiviruses, pictures of the cells were taken. The solid oval indicates healthy growth of normal fibroblast infected with shRNAs to CD320 and LRP2. The broken line ovals indicate unhealthy dying cancer cells infected with shRNAs targeting CD320 and LRP2 (FIG. 2A). The fields of cells in FIG. 2A were counted and quantified and illustrated in FIG. 2B. The data in FIG. 2B were normalized to the number of control cells and illustrated in FIG. 2C. FIG. 2C. shows that the cultures of cells infected with lentivirus encoding the shRNAs against CD320 and LRP2 (white bars) contain far fewer cells than the cultures of cells exposed to the shRNA control (black bar).

FIGS. 3A-F illustrate graphs of protein levels resulting from transfection of HEK293, MDA-MB-435S and MDA-MB-231 cells with siRNA to LRP2 and CD320. HEK293, MDA-MB-435S and MDA-MB-231 cells were transfected with 20 nM of indicated siRNAs and incubated for 48 hours. siRNAs targeting CD320 are designated OSC17 and OSC47. siRNAs targeting LRP2 are designated OSL245, OSL47, OSL104, OSL90 and OSL119. Whole cell lysates were prepared and immunoblotted for CD320 and LRP2 protein levels. The protein levels were normalized to a housekeeping control gene unaffected by the siRNA transfection. The graphs FIG. 3A-F represent the fold change of protein levels compared to siScramble (OSS1 or OSS2). (Average+/−SEM is shown, n=3).

FIGS. 4A-F illustrate a graph of cells after transfection of LnCAP, MCF-7 and U251 cells with siRNA to LRP2 and CD320. LnCAP, MCF-7 and U251 cells were transfected with 20 nM of indicated siRNAs and incubated for 48 hours. siRNAs targeting CD320 are designated OSC17 and OSC47. SiRNAs targeting LRP2 are designated OSL245, OSL47, OSL104, OSL90 and OSL119). Whole cell lysates were prepared and immunoblotted for CD320 and LRP2 protein levels. The protein levels were normalized to a housekeeping control gene unaffected by the siRNA transfection. The graphs FIG. 4A-F represent the fold change of protein levels compared to siScramble (OSS2).

FIGS. 5A-C illustrate graphs of protein levels after transfection of A172, DU145 and GM05659 cells with siRNA to LRP2 and CD320. A172, DU145 and GM05659 cells were transfected with 20 nM of indicated siRNAs and incubated for 48 hours. siRNAs targeting CD320 are designated OSC17 and OSC47. siRNAs targeting LRP2 are designated OSL245, OSL47, OSL104, OSL90 and OSL119). Whole cell lysates were prepared and immunoblotted for CD320. The protein levels were normalized to a housekeeping control gene unaffected by the siRNA transfection. The graphs FIG. 5A-C represent the fold change of protein levels compared to siScramble (OSS2).

FIG. 6 illustrates a graph of relative LRP2 protein expression in various cell lines-Lysates were made from the cell lines indicated on the x-axis, and western blot was performed to determine LRP2 protein levels. The results represent the averages+/−SEM of three independent lysates.

FIGS. 7A-B illustrates graphs of the effect of doxorubicin treatment on cell viability, as measured by the CTG assay. A172 and HCC15 cells were plated at 1200 cells/well in a 96 well plate. The next day, cells were treated with doxorubicin at the indicated concentrations. Four days after doxorubicin treatment was initiated, the cells were assayed for viability using the CTG assay. The dashed line indicates the non-linear fitting of the data to calculate an IC₅₀ value.

FIG. 8 is a schematic overview of the functional assay for screening siRNA effects on cell proliferation to facilitate quantification of the effects of knocking down CD320 and LRP2 on cell proliferation. Cells were plated in a 24-well plate. The next day, the cells were transfected with siRNAs targeting CD320 (OSC17, OSC47) and/or targeting LRP2 (OSL231, OSL245), or a control siRNA (OSS2). The cell lines may require repeated transfections and/or time for efficient toxicity (cell line dependent). In this experimental set-up there is room for repeat infection should some cell lines require that for efficient toxicity. In addition, in a small subset of the wells, cells were only treated with doxorubicin as a positive control for toxicity. At the end of the study, the cell lines are analyzed for cell growth by the CTG assay.

FIGS. 9A-E illustrate graphs of the percent cell survival of siCD320 and siLRP2 on cell proliferation-Cell lines representative of several types of cancers (lung, brain) or normal fibroblasts were transfected with individual or combinations of siRNAs targeting CD320 (OSC17, OSC47) or LRP2 (OSL231, OSL245), individually at 20 nM or in combination (10 nM each), or a negative control siRNA (OSS2) (20 nM) as indicated. Cells were repeatedly transfected as outlined in Table 9 for efficient toxicity, then assayed for viability by the CTG assay. Doxorubicin-treated cells served as a positive control for cell toxicity in our assays (Table 8).

FIGS. 10A-E illustrate graphs of the effects of siCD320 and siLRP2 on cell proliferation-Cell lines representative of several types of cancers (breast, prostate, skin) were transfected with individual or combinations of siRNAs targeting CD320 (OSC17, OSC47) or LRP2 (OSL231, OSL245) as indicated. Cells were repeatedly transfected as outlined in Table 9 for efficient toxicity, then assayed for viability by the CTG assay. Doxorubicin-treated cells served as a positive control for cell toxicity in our assays (Table 8).

FIGS. 11A-B illustrate the effects of siCD320 and siLRP2 molar proportions on cell proliferation with different molar proportions of siRNA targeting CD320 and siRNA targeting LRP2. Cell lines representative of two types of cancers (breast, prostate) were transfected with different proportions of siRNAs targeting CD320 (OSC17) or LRP2 (OSL245) (0-20 nM) or a negative control siRNA (OSS2) as indicated. Cells were repeatedly transfected for efficient toxicity then assayed for viability by the CTG assay. Doxorubicin-treated cells served as a positive control for cell toxicity in our assays (Table 8).

FIGS. 12A-B illustrate graphs of the duration of the knockdown effect for siCD320 and siLRP2 on MDA-MD-231 cells. A representative breast cancer cell line (MDA-MD-231) was transfected on Day 0 with 20 nM of an siRNA targeting CD320 (OSC17) or an siRNA targeting LRP2 (OSL245) or a negative control siRNA (OSS2) and the percentage of protein knockdown was analyzed daily over a period of five days by western blot. Protein levels were normalized to the negative control (OSS2).

FIG. 13 is a schematic of polyethylenimine (PEI) and siRNA complexation. PEI and siRNAs are mixed together. Subsequently, polyplexes (a nanoparticle, broadly speaking) of the PEI-siRNA complex form, which are able to enter the cell.

FIG. 14 is a schematic that illustrates that siRNAs are short RNA duplexes of generally 16 to 30 nucleotides; the guide sequence of the siRNA is complementary to a mRNA expressed in the cell. Exogenous siRNA duplexes are introduced into the cell via a method of transfection. The siRNA duplexes are separated via the RISC/AGO (RNA-induced silencing complex) complex, whereby the guide strand of the siRNA hybridizes with its complementary mRNA molecule. The mRNA is degraded by the RISC/AGO complex, which has RNAse activity, resulting in mRNA degradation, and the protein encoded by the mRNA is not produced. This causes the “knockdown” effect or reduced protein levels of the gene targeted by the siRNA compared to control treated cells.

FIGS. 15A-B illustrate graphs of A172 cell line or MDA-MD-435S cell lines treated with control siRNA (OSS1, OSS2) and siRNA directed to CD320 mRNA (OSC17, OSC47) and siRNA directed to LRP2 mRNA (OSL231, OSL245) to determine the effectiveness of INTERFERin, a polyethanolamine transfection reagent, in delivering siRNAs to cancer cells.

FIGS. 16A-D illustrate plated cells showing the effects of siCD320 and siLRP2 on four cell lines. Cell lines representative of four types of cancers (breast, two prostate, skin) were transfected with siRNAs targeting CD320 (OSC17) or LRP2 (OSL245) individually at 20 nM or in combination (10 nM each) or a negative control siRNA (OSS2) (20 nM) as indicated. Cells were repeatedly transfected for efficient toxicity as in Table 9 and then analyzed by microscopy as indicated.

FIG. 17 illustrates a graphical depiction of CD320 mRNA. UTR references the untranslated region, and the CDS references the protein coding sequence.

FIG. 18 illustrates a graphical depiction of LRP2 mRNA UTR references the untranslated region, and the CDS references the protein coding sequence.

FIGS. 19A-G illustrates the structures for unnatural nucleotides which may be incorporated within the sequence of an RNAi. “B” represents a natural (G, C, A, U) RNA nucleobase, a DNA nucleobase, or an unnatural nucleobase. FIG. 19A shows certain chemical modifications to the ribose 2′-position and phosphate moieties. FIG. 19B-D shows skeletal modifications to the ribose moiety that comprise bridging groups. FIG. 19E shows a deletion of the C2′-C3′ bond. FIG. 19F-G shows other skeletal modifications to the ribose moiety wherein a six-membered ring replaces the five-membered ring.

FIG. 20 illustrates a schematic for the in vivo murine xenograft model for breast cancer. MDA-MB-231 cells were implanted into the flank of NSG mice and grown to a volume of 70 mm³ after which siRNAs targeting CD320 (OSC17) and LRP2 (OSL245) were injected intratumorally once every fourth day.

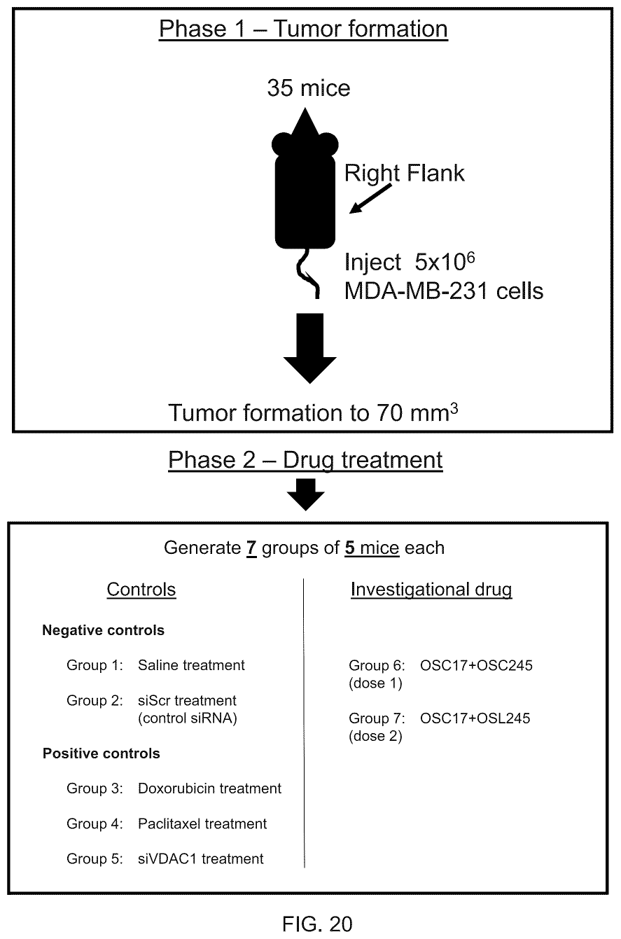

### DETAILED DESCRIPTION OF THE INVENTION

One or more embodiment of the present invention provides methods and RNAi compounds for modulating the expression of a CD320 gene and/or an LRP2 gene in a cell. In certain embodiments, expression of a CD320 gene and/or a LRP2 gene is reduced or inhibited using an CD320 and/or LRP2 specific RNAi. Such inhibition can be useful in treating disorders such as cancer and/or creating cell lines that are useful for screening drugs that treat cancer

The present invention also relates to a method for knocking down (partially or completely) the targeted genes.

One embodiment of the method of producing knockdown cells and organisms comprises introducing into a cell or organism in which a gene (referred to as a targeted gene) to be knocked down, an siRNA of about 16 to about 30 nucleotides (nt) that targets the gene and maintaining the resulting cell or organism under conditions under which RNAi occurs, resulting in degradation of the mRNA of the targeted gene, thereby producing knockdown cells or organisms. Knockdown cells and organisms produced by the present method are also the subject of embodiment of the present invention.

An embodiment of the present invention also relates to a method of examining or assessing the function of a gene in a cell or organism. In one embodiment, RNA of about 16 to about 30 nt which targets mRNA of the gene for degradation is introduced into a cell or organism in which RNAi occurs. The cell or organism is referred to as a test cell or organism. The cell or organism is referred to as a test cell organism. The test cell or organism is maintained under conditions under which degradation of mRNA of the gene occurs. The phenotype of the test cell or organism is then observed and compared to that of an appropriate control cell or organism, such as a corresponding cell or organism that is treated in the same manner except that the gene is not targeted. A 16 to 30 nt RNA that does not target the mRNA for degradation can be introduced into the control cell or organism in place of the siRNA introduced into the test cell or organism, although it is not necessary to do so. A difference between the phenotypes of the test and control cells or organisms provides information about the function of the degraded mRNA.

The RNA of about 16 to about 30 nucleotides is isolated or synthesized and then introduced into a cell or organism in which RNAi occurs (test cell or test organism). The test cell or test organism is maintained under conditions under which degradation of the mRNA occurs. The phenotype of the test cell or organism is then observed and compared to that of an appropriate control, such as a corresponding cell or organism that is treated in the same manner as the test cell or organism except that the targeted gene is not targeted. A difference between the phenotypes of the test and control cells or organisms provides information about the function of the targeted gene. The information provided may be sufficient to identify (define) the function of the gene or may be used in conjunction with information obtained from other assays or analyses to do so.

An embodiment of the present invention also encompasses a method of treating a disease or condition associated with the presence of a protein in an individual, comprising administering to the individual RNA of from about 16 to about 30 nucleotides which targets the mRNA of the protein (the mRNA that encodes the protein) for degradation. As a result, the protein is not produced or is not produced to the extent it would be in the absence of the treatment.

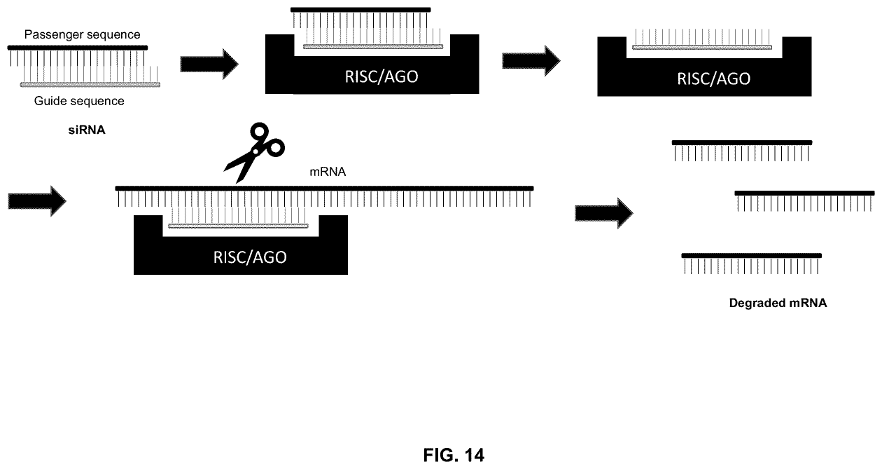

FIG. 14 shows that siRNAs are short RNA duplexes of generally 16 to 30 nucleotides; the sequence of the siRNA is complementary to a mRNA expressed in the cell. Exogenous siRNA duplexes are introduced into the cell via a method of transfection. The siRNA duplexes are unwound via the RNA-induced silencing complex (RISC), whereby the guide strand of the siRNA hybridizes with its complementary mRNA molecule. The mRNA is degraded by the RISC/AGO complex, which has RNAse cleave activity. The end result is that the mRNA targeted by the siRNA is degraded, and the protein encoded by the mRNA is not produced. This causes the “knockdown” effect or reduced protein levels of the gene targeted by the siRNA compared to control treated cells.

In one embodiment, at least one strand of the RNA molecule has a 3′ overhang from about 1 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length or, for example, the overhang can be up to 14 nucleotides if the guide strand were a 27-mer. In one embodiment the RNA molecule is double stranded, one strand has a 3′ overhang and the other strand can be blunt-ended or have an overhang. In the embodiment in which the RNA molecule is double stranded and both strands comprise an overhang, the length of the overhangs may be the same or different for each strand. In a particular embodiment, the RNA of the present invention comprises 21-27 nucleotide strands which are Watson-Crick paired and which have overhangs of from about 1 to about 3, particularly about 2, nucleotides on both 3′ ends of the RNA. In order to further enhance the stability of the RNA of the present invention, the 3′ overhangs can be stabilized against degradation. In one embodiment, the RNA is stabilized by including purine nucleotides, such as adenosine or guanosine nucleotides. Alternatively, substitution of pyrimidine nucleotides by unnatural nucleotides, e.g., substitution of uridine 2 nucleotide 3′ overhangs by 2′-deoxythymidine, is tolerated and does not affect the efficiency of RNAi. The absence of a 2′ hydroxyl significantly enhances the nuclease resistance of the overhang in tissue culture medium. The 3′-overhangs can be further stabilized by introduction of phosphorothioate groups in place of the phosphodiesters.

The 16-30 nt RNA molecules of the present invention can be obtained using a number of techniques known to those of skill in the art. For example, the RNA can be chemically synthesized or recombinantly produced using methods known in the art.

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

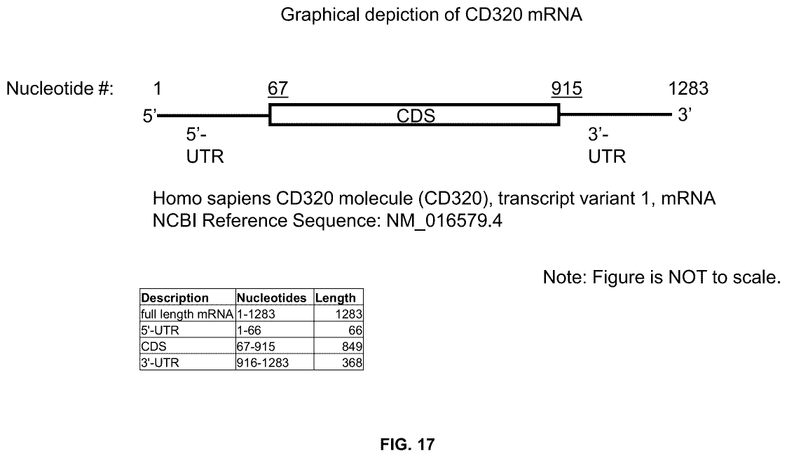

As used herein, “CD320” refers to the gene or protein. CD320 is also known as 8D6 antigen, CD320 antigen, 8D6A, transcobalamin receptor, FDC-SM-8D6, FDC-Signaling Molecule 8D6, 8D6, TCBLR, TCbIR, TCN2R. The term CD320 includes human CD320, the amino acid and nucleotide sequence of which may be found in, for example, GenBank Accession No. NM_016579.4 and NM_001165895.2; mouse CD320, the amino acid and nucleotide sequence of which may be found in, for example, GenBank Accession No. NM_019421.3; rat CD320, the amino acid and nucleotide sequence of which may be found in, for example, GenBank Accession No. NM_001014201.1. Additional examples of CD320 mRNA sequences are readily available using, e.g., GenBank. Additional information is found at FIG. 17.

The CD320 DNA sequence from Homo sapiens is as follows: >NM_016579.4 Homo sapiens CD320 molecule (CD320), transcript variant 1, DNA

(SEQ ID NO. 935)
GTGCGCGTGCGCAGGGATAAGAGAGCGGTCTGGACAGCGCGTGGCCGGC
GCCGCTGTGGGGACAGCATGAGCGGCGGTTGGATGGCGCAGGTTGGAGC
GTGGCGAACAGGGGCTCTGGGCCTGGCGCTGCTGCTGCTGCTCGGCCTC
GGACTAGGCCTGGAGGCCGCCGCGAGCCCGCTTTCCACCCCGACCTCTG
CCCAGGCCGCAGGCCCCAGCTCAGGCTCGTGCCCACCCACCAAGTTCCA
GTGCCGCACCAGTGGCTTATGCGTGCCCCTCACCTGGCGCTGCGACAGG
GACTTGGACTGCAGCGATGGCAGCGATGAGGAGGAGTGCAGGATTGAGC
CATGTACCCAGAAAGGGCAATGCCCACCGCCCCCTGGCCTCCCCTGCCC
CTGCACCGGCGTCAGTGACTGCTCTGGGGGAACTGACAAGAAACTGCGC
AACTGCAGCCGCCTGGCCTGCCTAGCAGGCGAGCTCCGTTGCACGCTGA
GCGATGACTGCATTCCACTCACGTGGCGCTGCGACGGCCACCCAGACTG
TCCCGACTCCAGCGACGAGCTCGGCTGTGGAACCAATGAGATCCTCCCG
GAAGGGGATGCCACAACCATGGGGCCCCCTGTGACCCTGGAGAGTGTCA
CCTCTCTCAGGAATGCCACAACCATGGGGCCCCCTGTGACCCTGGAGAG
TGTCCCCTCTGTCGGGAATGCCACATCCTCCTCTGCCGGAGACCAGTCT
GGAAGCCCAACTGCCTATGGGGTTATTGCAGCTGCTGCGGTGCTCAGTG
CAAGCCTGGTCACCGCCACCCTCCTCCTTTTGTCCTGGCTCCGAGCCCA
GGAGCGCCTCCGCCCACTGGGGTTACTGGTGGCCATGAAGGAGTCCCTG
CTGCTGTCAGAACAGAAGACCTCGCTGCCCTGAGGACAAGCACTTGCCA
CCACCGTCACTCAGCCCTGGGCGTAGCCGGACAGGAGGAGAGCAGTGAT
GCGGATGGGTACCCGGGCACACCAGCCCTCAGAGACCTGAGCTCTTCTG
GCCACGTGGAACCTCGAACCCGAGCTCCTGCAGAAGTGGCCCTGGAGAT
TGAGGGTCCCTGGACACTCCCTATGGAGATCCGGGGAGCTAGGATGGGG
AACCTGCCACAGCCAGAACTGAGGGGCTGGCCCCAGGCAGCTCCCAGGG
GGTAGAACGGCCCTGTGCTTAAGACACTCCTGCTGCCCCGTCTGAGGGT
GGCGATTAAAGTTGCTTCACATCCTCAAAAAAAAAAAAAAAAAAAAAAA
AAAAAAAAA.

A protein sequence from CD320 derived from the mRNA sequence above is as follows:

(SEQ ID NO. 936)
>sp|Q9NPF0|CD320_HUMAN CD320 antigen OS = Homo
sapiens OX = 9606 GN = CD320 PE = 1 SV = 1
MSGGWMAQVGAWRTGALGLALLLLLGLGLGLEAAASPLSTPTSAQAAGP
SSGSCPPTKFQCRTSGLCVPLTWRCDRDLDCSDGSDEEECRIEPCTQKG
QCPPPPGLPCPCTGVSDCSGGTDKKLRNCSRLACLAGELRCTLSDDCIP
LTWRCDGHPDCPDSSDELGCGTNEILPEGDATTMGPPVTLESVTSLRNA
TTMGPPVTLESVPSVGNATSSSAGDQSGSPTAYGVIAAAAVLSASLVTA
TLLLLSWLRAQERLRPLGLLVAMKESLLLSEQKTSLP

The CD320 DNA sequence from Homo sapiens is as follows: >NM_001165895.2 Homo sapiens CD320 molecule (CD320), transcript variant 2, DNA

(SEQ ID NO. 937)
GCGTGCGCGTGCGCAGGGATAAGAGAGCGGTCTGGACAGCGCGTGGCCG
GCGCCGCTGTGGGGACAGCATGAGCGGCGGTTGGATGGCGCAGGTTGGA
GCGTGGCGAACAGGGGCTCTGGGCCTGGCGCTGCTGCTGCTGCTCGGCC
TCGGACTAGGCCTGGAGGCCGCCGCGAGCCCGCTTTCCACCCCGACCTC
TGCCCAGGCCGCAGGGATTGAGCCATGTACCCAGAAAGGGCAATGCCCA
CCGCCCCCTGGCCTCCCCTGCCCCTGCACCGGCGTCAGTGACTGCTCTG
GGGGAACTGACAAGAAACTGCGCAACTGCAGCCGCCTGGCCTGCCTAGC
AGGCGAGCTCCGTTGCACGCTGAGCGATGACTGCATTCCACTCACGTGG
CGCTGCGACGGCCACCCAGACTGTCCCGACTCCAGCGACGAGCTCGGCT
GTGGAACCAATGAGATCCTCCCGGAAGGGGATGCCACAACCATGGGGCC
CCCTGTGACCCTGGAGAGTGTCACCTCTCTCAGGAATGCCACAACCATG
GGGCCCCCTGTGACCCTGGAGAGTGTCCCCTCTGTCGGGAATGCCACAT
CCTCCTCTGCCGGAGACCAGTCTGGAAGCCCAACTGCCTATGGGGTTAT
TGCAGCTGCTGCGGTGCTCAGTGCAAGCCTGGTCACCGCCACCCTCCTC
CTTTTGTCCTGGCTCCGAGCCCAGGAGCGCCTCCGCCCACTGGGGTTAC
TGGTGGCCATGAAGGAGTCCCTGCTGCTGTCAGAACAGAAGACCTCGCT
GCCCTGAGGACAAGCACTTGCCACCACCGTCACTCAGCCCTGGGCGTAG
CCGGACAGGAGGAGAGCAGTGATGCGGATGGGTACCCGGGCACACCAGC
CCTCAGAGACCTGAGCTCTTCTGGCCACGTGGAACCTCGAACCCGAGCT
CCTGCAGAAGTGGCCCTGGAGATTGAGGGTCCCTGGACACTCCCTATGG
AGATCCGGGGAGCTAGGATGGGGAACCTGCCACAGCCAGAACTGAGGGG
CTGGCCCCAGGCAGCTCCCAGGGGGTAGAACGGCCCTGTGCTTAAGACA
CTCCTGCTGCCCCGTCTGAGGGTGGCAATTAAAGTTGCTTCACATCCTC

A protein sequence from CD320 derived from the DNA sequence above is as follows:

(SEQ ID NO. 938)
>sp|Q9NPF0-2|CD320_HUMAN Isoform 2 of CD320
antigen OS = Homo sapiens OX = 9606 GN = CD320
MSGGWMAQVGAWRTGALGLALLLLLGLGLGLEAAASPLSTPTSAQAAGI
EPCTQKGQCPPPPGLPCPCTGVSDCSGGTDKKLRNCSRLACLAGELRCT
LSDDCIPLTWRCDGHPDCPDSSDELGCGTNEILPEGDATTMGPPVTLES
VTSLRNATTMGPPVTLESVPSVGNATSSSAGDQSGSPTAYGVIAAAAVL
SASLVTATLLLLSWLRAQERLRPLGLLVAMKESLLLSEQKTSLP

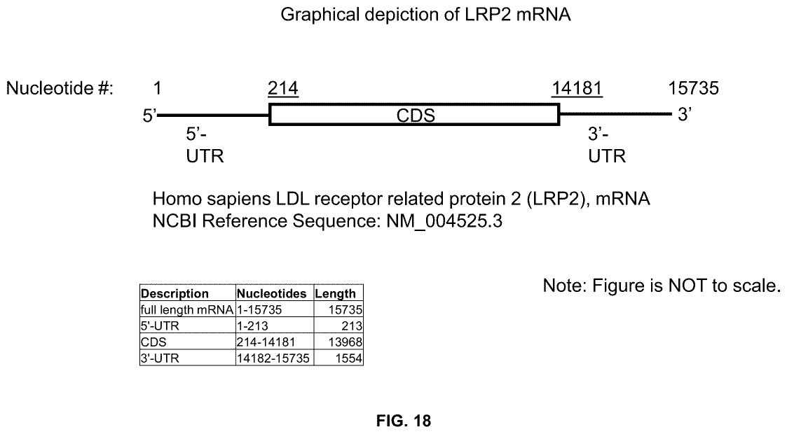

Further, as used herein, “LRP2” refers to the gene or protein. LRP2 is also known as megalin, LRP-2, Glycoprotein 330, DBS, GP330, Gp330, Calcium Sensor Protein, Heymann Nephritis Antigen Homolog, Low-Density Lipoprotein Receptor-Related Protein 2, EC 1.1.2.3, EC 3.4.21.9, LDL receptor related protein 2. The term LRP2 includes human LRP2, the amino acid and nucleotide sequence of which may be found in, for example, GenBank Accession No. NM_004525.3; mouse LRP2, the amino acid and nucleotide sequence of which may be found in, for example, GenBank Accession No. NM_001081088.2; rat LRP2, the amino acid and nucleotide sequence of which may be found in, for example, GenBank Accession No. NM_030827.1. Additional examples of LRP2 mRNA sequences are readily available using, e.g., GenBank. Additional information is found at FIG. 18.

One example of LRP2 is: >NM_004525.3 Homo sapiens LDL receptor related protein 2 (LRP2), DNA:

(SEQ ID NO. 939)
GGTCTAAAGGGCTTTATGCACTGTCTGGAGGGTGGGGACTGGCGCGGGTAGAAAACGGGATGCCTCGGGC
GTGGGGGCAGGCTTTTGGCCACTAGGAGCTGGCGGAGGTGCAGACCTAAAGGAGCGTTCGCTAGCAGAGG
CGCTGCCGGTGCGGTGTGCTACGCGCGCCCACCTCCCGGGGAAGGAACGGCGAGGCCGGGGACCGTCGCG
GAGATGGATCGCGGGCCGGCAGCAGTGGCGTGCACGCTGCTCCTGGCTCTCGTCGCCTGCCTAGCGCCGG
CCAGTGGCCAAGAATGTGACAGTGCGCATTTTCGCTGTGGAAGTGGGCATTGCATCCCTGCAGACTGGAG
GTGTGATGGGACCAAAGACTGTTCAGATGACGCGGATGAAATTGGCTGCGCTGTTGTGACCTGCCAGCAG
GGCTATTTCAAGTGCCAGAGTGAGGGACAATGCATCCCCAACTCCTGGGTGTGTGACCAAGATCAAGACT
GTGATGATGGCTCAGATGAACGTCAAGATTGCTCACAAAGTACATGCTCAAGTCATCAGATAACATGCTC
CAATGGTCAGTGTATCCCAAGTGAATACAGGTGCGACCACGTCAGAGACTGCCCCGATGGAGCTGATGAG
AATGACTGCCAGTACCCAACATGTGAGCAGCTTACTTGTGACAATGGGGCCTGCTATAACACCAGTCAGA
AGTGTGATTGGAAAGTTGATTGCAGGGACTCCTCAGATGAAATCAACTGCACTGAGATATGCTTGCACAA
TGAGTTTTCATGTGGCAATGGAGAGTGTATCCCTCGTGCTTATGTCTGTGACCATGACAATGATTGCCAA
GACGGCAGTGACGAACATGCTTGCAACTATCCGACCTGCGGTGGTTACCAGTTCACTTGCCCCAGTGGCC
GATGCATTTATCAAAACTGGGTTTGTGATGGAGAAGATGACTGTAAAGATAATGGAGATGAAGATGGATG
TGAAAGCGGTCCTCATGATGTTCATAAATGTTCCCCAAGAGAATGGTCTTGCCCAGAGTCGGGACGATGC
ATCTCCATTTATAAAGTTTGTGATGGGATTTTAGATTGCCCAGGAAGAGAAGATGAAAACAACACTAGTA
CCGGAAAATACTGTAGTATGACTCTGTGCTCTGCCTTGAACTGCCAGTACCAGTGCCATGAGACGCCGTA
TGGAGGAGCGTGTTTTTGTCCCCCAGGTTATATCATCAACCACAATGACAGCCGTACCTGTGTTGAGTTT
GATGATTGCCAGATATGGGGAATTTGTGACCAGAAGTGTGAAAGCCGACCTGGCCGTCACCTGTGCCACT
GTGAAGAAGGGTATATCTTGGAGCGTGGACAGTATTGCAAAGCTAATGATTCCTTTGGCGAGGCCTCCAT
TATCTTCTCCAATGGTCGGGATTTGTTAATTGGTGATATTCATGGAAGGAGCTTCCGGATCCTAGTGGAG
TCTCAGAATCGTGGAGTGGCCGTGGGTGTGGCTTTCCACTATCACCTGCAAAGAGTTTTTTGGACAGACA
CCGTGCAAAATAAGGTTTTTTCAGTTGACATTAATGGTTTAAATATCCAAGAGGTTCTCAATGTTTCTGT
TGAAACCCCAGAGAACCTGGCTGTGGACTGGGTTAATAATAAAATCTATCTAGTGGAAACCAAGGTCAAC
CGCATAGATATGGTAAATTTGGATGGAAGCTATCGGGTTACCCTTATAACTGAAAACTTGGGGCATCCTA
GAGGAATTGCCGTGGACCCAACTGTTGGTTATTTATTTTTCTCAGATTGGGAGAGCCTTTCTGGGGAACC
TAAGCTGGAAAGGGCATTCATGGATGGCAGCAACCGTAAAGACTTGGTGAAAACAAAGCTGGGATGGCCT
GCTGGGGTAACTCTGGATATGATATCGAAGCGTGTTTACTGGGTTGACTCTCGGTTTGATTACATTGAAA
CTGTAACTTATGATGGAATTCAAAGGAAGACTGTAGTTCATGGAGGCTCCCTCATTCCTCATCCCTTTGG
AGTAAGCTTATTTGAAGGTCAGGTGTTCTTTACAGATTGGACAAAGATGGCCGTGCTGAAGGCAAACAAG
TTCACAGAGACCAACCCACAAGTGTACTACCAGGCTTCCCTGAGGCCCTATGGAGTGACTGTTTACCATT
CCCTCAGACAGCCCTATGCTACCAATCCGTGTAAAGATAACAATGGGGGCTGTGAGCAGGTCTGTGTCCT
CAGCCACAGAACAGATAATGATGGTTTGGGTTTCCGTTGCAAGTGCACATTCGGCTTCCAACTGGATACA
GATGAGCGCCACTGCATTGCTGTTCAGAATTTCCTCATTTTTTCATCCCAAGTTGCTATTCGTGGGATCC
CGTTCACCTTGTCTACCCAGGAAGATGTCATGGTTCCAGTTTCGGGGAATCCTTCTTTCTTTGTCGGGAT
TGATTTTGACGCCCAGGACAGCACTATCTTTTTTTCAGATATGTCAAAACACATGATTTTTAAGCAAAAG
ATTGATGGCACAGGAAGAGAAATTCTCGCAGCTAACAGGGTGGAAAATGTTGAAAGTTTGGCTTTTGATT
GGATTTCAAAGAATCTCTATTGGACAGACTCTCATTACAAGAGTATCAGTGTCATGAGGCTAGCTGATAA
AACGAGACGCACAGTAGTTCAGTATTTAAATAACCCACGGTCGGTGGTAGTTCATCCTTTTGCCGGGTAT
CTATTCTTCACTGATTGGTTCCGTCCTGCTAAAATTATGAGAGCATGGAGTGACGGATCTCACCTCTTGC
CTGTAATAAACACTACTCTTGGATGGCCCAATGGCTTGGCCATCGATTGGGCTGCTTCACGATTGTACTG
GGTAGATGCCTATTTTGATAAAATTGAGCACAGCACCTTTGATGGTTTAGACAGAAGAAGACTGGGCCAT
ATAGAGCAGATGACACATCCGTTTGGACTTGCCATCTTTGGAGAGCATTTATTTTTTACTGACTGGAGAC
TGGGTGCCATTATTCGAGTCAGGAAAGCAGATGGTGGAGAAATGACAGTTATCCGAAGTGGCATTGCTTA
CATACTGCATTTGAAATCGTATGATGTCAACATCCAGACTGGTTCTAACGCCTGTAATCAACCCACGCAT
CCTAACGGTGACTGCAGCCACTTCTGCTTCCCGGTGCCAAATTTCCAGCGAGTGTGTGGGTGCCCTTATG
GAATGAGGCTGGCTTCCAATCACTTGACATGCGAGGGGGACCCAACCAATGAACCACCCACAGAGCAGTG
TGGCTTATTTTCCTTCCCCTGTAAAAATGGCAGATGTGTGCCCAATTACTATCTCTGTGATGGAGTCGAT
GATTGTCATGATAACAGTGATGAGCAACTATGTGGCACACTTAATAATACCTGTTCATCTTCGGCGTTCA
CCTGTGGCCATGGGGAGTGCATTCCTGCACACTGGCGCTGTGACAAACGCAACGACTGTGTGGATGGCAG
TGATGAGCACAACTGCCCCACCCACGCACCTGCTTCCTGCCTTGACACCCAATACACCTGTGATAATCAC
CAGTGTATCTCAAAGAACTGGGTCTGTGACACAGACAATGATTGTGGGGATGGATCTGATGAAAAGAACT
GCAATTCGACAGAGACATGCCAACCTAGTCAGTTTAATTGCCCCAATCATCGATGTATTGACCTATCGTT
TGTCTGTGATGGTGACAAGGATTGTGTTGATGGATCTGATGAGGTTGGTTGTGTATTAAACTGTACTGCT
TCTCAATTCAAGTGTGCCAGTGGGGATAAATGTATTGGCGTCACAAATCGTTGTGATGGTGTTTTTGATT
GCAGTGACAACTCGGATGAAGCAGGCTGTCCAACCAGGCCTCCTGGTATGTGCCACTCAGATGAATTTCA
GTGCCAAGAAGATGGTATCTGCATCCCGAACTTCTGGGAATGTGATGGGCATCCAGACTGCCTCTATGGA
TCTGATGAGCACAATGCCTGTGTCCCCAAGACTTGCCCTTCATCATATTTCCACTGTGACAACGGAAACT
GCATCCACAGGGCATGGCTCTGTGATCGGGACAATGACTGCGGGGATATGAGTGATGAGAAGGACTGCCC
TACTCAGCCCTTTCGCTGTCCTAGTTGGCAATGGCAGTGTCTTGGCCATAACATCTGTGTGAATCTGAGT
GTAGTGTGTGATGGCATCTTTGACTGCCCCAATGGGACAGATGAGTCCCCACTTTGCAATGGGAACAGCT
GCTCAGATTTCAATGGTGGTTGTACTCACGAGTGTGTTCAAGAGCCCTTTGGGGCTAAATGCCTATGTCC
ATTGGGATTCTTACTTGCCAATGATTCTAAGACCTGTGAAGACATAGATGAATGTGATATTCTAGGCTCT
TGTAGCCAGCACTGTTACAATATGAGAGGTTCTTTCCGGTGCTCGTGTGATACAGGCTACATGTTAGAAA
GTGATGGGAGGACTTGCAAAGTTACAGCATCTGAGAGTCTGCTGTTACTTGTGGCAAGTCAGAACAAAAT
TATTGCCGACAGTGTCACCTCCCAGGTCCACAATATCTATTCATTGGTCGAGAATGGTTCTTACATTGTA
GCTGTTGATTTTGATTCAATTAGTGGTCGTATCTTTTGGTCTGATGCAACTCAGGGTAAAACCTGGAGTG
CGTTTCAAAATGGAACGGACAGAAGAGTGGTATTTGACAGTAGCATCATCTTGACTGAAACTATTGCAAT
AGATTGGGTAGGTCGTAATCTTTACTGGACAGACTATGCTCTGGAAACAATTGAAGTCTCCAAAATTGAT
GGGAGCCACAGGACTGTGCTGATTAGTAAAAACCTAACAAATCCAAGAGGACTAGCATTAGATCCCAGAA
TGAATGAGCATCTACTGTTCTGGTCTGACTGGGGCCACCACCCTCGCATCGAGCGAGCCAGCATGGACGG
CAGCATGCGCACTGTCATTGTCCAGGACAAGATCTTCTGGCCCTGCGGCTTAACTATTGACTACCCCAAC
AGACTGCTCTACTTCATGGACTCCTATCTTGATTACATGGACTTTTGTGATTATAATGGACACCATCGGA
GACAGGTGATAGCCAGTGATTTGATTATACGGCACCCCTATGCCCTAACTCTCTTTGAAGACTCTGTGTA
CTGGACTGACCGTGCTACTCGTCGGGTTATGCGAGCCAACAAGTGGCATGGAGGGAACCAGTCAGTTGTA
ATGTATAATATTCAATGGCCCCTTGGGATTGTTGCGGTTCATCCTTCGAAACAACCAAATTCCGTGAATC
CATGTGCCTTTTCCCGCTGCAGCCATCTCTGCCTGCTTTCCTCACAGGGGCCTCATTTTTACTCCTGTGT
TTGTCCTTCAGGATGGAGTCTGTCTCCTGATCTCCTGAATTGCTTGAGAGATGATCAACCTTTCTTAATA
ACTGTAAGGCAACATATAATTTTTGGAATCTCCCTTAATCCTGAGGTGAAGAGCAATGATGCTATGGTCC
CCATAGCAGGGATACAGAATGGTTTAGATGTTGAATTTGATGATGCTGAGCAATACATCTATTGGGTTGA
AAATCCAGGTGAAATTCACAGAGTGAAGACAGATGGCACCAACAGGACAGTATTTGCTTCTATATCTATG
GTGGGGCCTTCTATGAACCTGGCCTTAGATTGGATTTCAAGAAACCTTTATTCTACCAATCCTAGAACTC
AGTCAATCGAGGTTTTGACACTCCACGGAGATATCAGATACAGAAAAACATTGATTGCCAATGATGGGAC
AGCTCTTGGAGTTGGCTTTCCAATTGGCATAACTGTTGATCCTGCTCGTGGGAAGCTGTACTGGTCAGAC
CAAGGAACTGACAGTGGGGTTCCTGCCAAGATCGCCAGTGCTAACATGGATGGCACATCTGTGAAAACTC
TCTTTACTGGGAACCTCGAACACCTGGAGTGTGTCACTCTTGACATCGAAGAGCAGAAACTCTACTGGGC
AGTCACTGGAAGAGGAGTGATTGAAAGAGGAAACGTGGATGGAACAGATCGAATGATCCTGGTACACCAG
CTTTCCCACCCCTGGGGAATTGCAGTCCATGATTCTTTCCTTTATTATACTGATGAACAGTATGAGGTCA
TTGAAAGAGTTGATAAGGCCACTGGGGCCAACAAAATAGTCTTGAGAGATAATGTTCCAAATCTGAGGGG
TCTTCAAGTTTATCACAGACGCAATGCCGCCGAATCCTCAAATGGCTGTAGCAACAACATGAATGCCTGT
CAGCAGATTTGCCTGCCTGTACCAGGAGGATTGTTTTCCTGCGCCTGTGCCACTGGATTTAAACTCAATC
CTGATAATCGGTCCTGCTCTCCATATAACTCTTTCATTGTTGTTTCAATGCTGTCTGCAATCAGAGGCTT
TAGCTTGGAATTGTCAGATCATTCAGAAACCATGGTGCCGGTGGCAGGCCAAGGACGAAACGCACTGCAT
GTGGATGTGGATGTGTCCTCTGGCTTTATTTATTGGTGTGATTTTAGCAGCTCAGTGGCATCTGATAATG
CGATCCGTAGAATTAAACCAGATGGATCTTCTCTGATGAACATTGTGACACATGGAATAGGAGAAAATGG
AGTCCGGGGTATTGCAGTGGATTGGGTAGCAGGAAATCTTTATTTCACCAATGCCTTTGTTTCTGAAACA
CTGATAGAAGTTCTGCGGATCAATACTACTTACCGCCGTGTTCTTCTTAAAGTCACAGTGGACATGCCTA
GGCATATTGTTGTAGATCCCAAGAACAGATACCTCTTCTGGGCTGACTATGGGCAGAGACCAAAGATTGA
GCGTTCTTTCCTTGACTGTACCAATCGAACAGTGCTTGTGTCAGAGGGCATTGTCACACCACGGGGCTTG
GCAGTGGACCGAAGTGATGGCTACGTTTATTGGGTTGATGATTCTTTAGATATAATTGCAAGGATTCGTA
TCAATGGAGAGAACTCTGAAGTGATTCGTTATGGCAGTCGTTACCCAACTCCTTATGGCATCACTGTTTT
TGAAAATTCTATCATATGGGTAGATAGGAATTTGAAAAAGATCTTCCAAGCCAGCAAGGAACCAGAGAAC
ACAGAGCCACCCACAGTGATAAGAGACAATATCAACTGGCTAAGAGATGTGACCATCTTTGACAAGCAAG
TCCAGCCCCGGTCACCAGCAGAGGTCAACAACAACCCTTGCTTGGAAAACAATGGTGGGTGCTCTCATCT
CTGCTTTGCTCTGCCTGGATTGCACACCCCAAAATGTGACTGTGCCTTTGGGACCCTGCAAAGTGATGGC
AAGAATTGTGCCATTTCAACAGAAAATTTCCTCATCTTTGCCTTGTCTAATTCCTTGAGAAGCTTACACT
TGGACCCTGAAAACCATAGCCCACCTTTCCAAACAATAAATGTGGAAAGAACTGTCATGTCTCTAGACTA
TGACAGTGTAAGTGATAGAATCTACTTCACACAAAATTTAGCCTCTGGAGTTGGACAGATTTCCTATGCC
ACCCTGTCTTCAGGGATCCATACTCCAACTGTCATTGCTTCAGGTATAGGGACTGCTGATGGCATTGCCT
TTGACTGGATTACTAGAAGAATTTATTACAGTGACTACCTCAACCAGATGATTAATTCCATGGCTGAAGA
TGGGTCTAACCGCACTGTGATAGCCCGCGTTCCAAAACCAAGAGCAATTGTGTTAGATCCCTGCCAAGGG
TACCTGTACTGGGCTGACTGGGATACACATGCCAAAATCGAGAGAGCCACATTGGGAGGAAACTTCCGCG
TACCCATTGTGAACAGCAGTCTGGTCATGCCCAGTGGGCTGACTCTGGACTATGAAGAGGACCTTCTCTA
CTGGGTGGATGCTAGTCTGCAGAGGATTGAACGCAGCACTCTGACGGGCGTGGATCGTGAAGTCATTGTC
AATGCAGCCGTTCATGCTTTTGGCTTGACTCTCTATGGCCAGTATATTTACTGGACTGACTTGTACACAC
AAAGAATTTACCGAGCTAACAAATATGACGGGTCAGGTCAGATTGCAATGACCACAAATTTGCTCTCCCA
GCCCAGGGGAATCAACACTGTTGTGAAGAACCAGAAACAACAGTGTAACAATCCTTGTGAACAGTTTAAT
GGGGGCTGCAGCCATATCTGTGCACCAGGTCCAAATGGTGCCGAGTGCCAGTGTCCACATGAGGGCAACT
GGTATTTGGCCAACAACAGGAAGCACTGCATTGTGGACAATGGTGAACGATGTGGTGCATCTTCCTTCAC
CTGCTCCAATGGGCGCTGCATCTCGGAAGAGTGGAAGTGTGATAATGACAACGACTGTGGGGATGGCAGT
GATGAGATGGAAAGTGTCTGTGCACTTCACACCTGCTCACCGACAGCCTTCACCTGTGCCAATGGGCGAT
GTGTCCAATACTCTTACCGCTGTGATTACTACAATGACTGTGGTGATGGCAGTGATGAGGCAGGGTGCCT
GTTCAGGGACTGCAATGCCACCACGGAGTTTATGTGCAATAACAGAAGGTGCATACCTCGTGAGTTTATC
TGCAATGGTGTAGACAACTGCCATGATAATAACACTTCAGATGAGAAAAATTGCCCTGATCGCACTTGCC
AGTCTGGATACACAAAATGTCATAATTCAAATATTTGTATTCCTCGCGTTTATTTGTGTGACGGAGACAA
TGACTGTGGAGATAACAGTGATGAAAACCCTACTTATTGCACCACTCACACGTGCAGCAGCAGTGAGTTC
CAATGCGCATCTGGGCGCTGTATTCCTCAACATTGGTATTGTGATCAAGAAACAGATTGTTTTGATGCCT
CTGATGAACCTGCCTCTTGTGGTCACTCTGAGCGAACATGCCTAGCTGATGAGTTCAAGTGTGATGGTGG
GAGGTGCATCCCAAGCGAATGGATCTGTGACGGTGATAATGACTGTGGGGATATGAGTGACGAGGATAAA
AGGCACCAGTGTCAGAATCAAAACTGCTCGGATTCCGAGTTTCTCTGTGTAAATGACAGACCTCCGGACA
GGAGGTGCATTCCCCAGTCTTGGGTCTGTGATGGCGATGTGGATTGTACTGACGGCTACGATGAGAATCA
GAATTGCACCAGGAGAACTTGCTCTGAAAATGAATTCACCTGTGGTTACGGACTGTGTATCCCAAAGATA
TTCAGGTGTGACCGGCACAATGACTGTGGTGACTATAGCGACGAGAGGGGCTGCTTATACCAGACTTGCC
AACAGAATCAGTTTACCTGTCAGAACGGGCGCTGCATTAGTAAAACCTTCGTCTGTGATGAGGATAATGA
CTGTGGAGACGGATCTGATGAGCTGATGCACCTGTGCCACACCCCAGAACCCACGTGTCCACCTCACGAG
TTCAAGTGTGACAATGGGCGCTGCATCGAGATGATGAAACTCTGCAACCACCTAGATGACTGTTTGGACA
ACAGCGATGAGAAAGGCTGTGGCATTAATGAATGCCATGACCCTTCAATCAGTGGCTGCGATCACAACTG
CACAGACACCTTAACCAGTTTCTATTGTTCCTGTCGTCCTGGTTACAAGCTCATGTCTGACAAGCGGACT
TGTGTTGATATTGATGAATGCACAGAGATGCCTTTTGTCTGTAGCCAGAAGTGTGAGAATGTAATAGGCT
CCTACATCTGTAAGTGTGCCCCAGGCTACCTCCGAGAACCAGATGGAAAGACCTGCCGGCAAAACAGTAA
CATCGAACCCTATCTCATTTTTAGCAACCGTTACTATTTGAGAAATTTAACTATAGATGGCTATTTTTAC
TCCCTCATCTTGGAAGGACTGGACAATGTTGTGGCATTAGATTTTGACCGAGTAGAGAAGAGATTGTATT
GGATTGATACACAGAGGCAAGTCATTGAGAGAATGTTTCTGAATAAGACAAACAAGGAGACAATCATAAA
CCACAGACTACCAGCTGCAGAAAGTCTGGCTGTAGACTGGGTTTCCAGAAAGCTCTACTGGTTGGATGCC
CGCCTGGATGGCCTCTTTGTCTCTGACCTCAATGGTGGACACCGCCGCATGCTGGCCCAGCACTGTGTGG
ATGCCAACAACACCTTCTGCTTTGATAATCCCAGAGGACTTGCCCTTCACCCTCAATATGGGTACCTCTA
CTGGGCAGACTGGGGTCACCGCGCATACATTGGGAGAGTAGGCATGGATGGAACCAACAAGTCTGTGATA
ATCTCCACCAAGTTAGAGTGGCCTAATGGCATCACCATTGATTACACCAATGATCTACTCTACTGGGCAG
ATGCCCACCTGGGTTACATAGAGTACTCTGATTTGGAGGGCCACCATCGACACACGGTGTATGATGGGGC
ACTGCCTCACCCTTTCGCTATTACCATTTTTGAAGACACTATTTATTGGACAGATTGGAATACAAGGACA
GTGGAAAAGGGAAACAAATATGATGGATCAAATAGACAGACACTGGTGAACACAACACACAGACCATTTG
ACATCCATGTGTACCATCCATATAGGCAGCCCATTGTGAGCAATCCCTGTGGTACCAACAATGGTGGCTG
TTCTCATCTCTGCCTCATCAAGCCAGGAGGAAAAGGGTTCACTTGCGAGTGTCCAGATGACTTCCGCACC
CTTCAGCTGAGTGGCAGCACCTACTGCATGCCCATGTGCTCCAGCACCCAGTTCCTGTGCGCTAACAATG
AAAAGTGCATTCCTATCTGGTGGAAATGTGATGGACAGAAAGACTGCTCAGATGGCTCTGATGAACTGGC
CCTTTGCCCGCAGCGCTTCTGCCGACTGGGACAGTTCCAGTGCAGTGACGGCAACTGCACCAGCCCGCAG
ACTTTATGCAATGCTCACCAAAATTGCCCTGATGGGTCTGATGAAGACCGTCTTCTTTGTGAGAATCACC
ACTGTGACTCCAATGAATGGCAGTGCGCCAACAAACGTTGCATCCCAGAATCCTGGCAGTGTGACACATT
TAACGACTGTGAGGATAACTCAGATGAAGACAGTTCCCACTGTGCCAGCAGGACCTGCCGGCCGGGCCAG
TTTCGGTGTGCTAATGGCCGCTGCATCCCGCAGGCCTGGAAGTGTGATGTGGATAATGATTGTGGAGACC
ACTCGGATGAGCCCATTGAAGAATGCATGAGCTCTGCCCATCTCTGTGACAACTTCACAGAATTCAGCTG
CAAAACAAATTACCGCTGCATCCCAAAGTGGGCCGTGTGCAATGGTGTAGATGACTGCAGGGACAACAGT
GATGAGCAAGGCTGTGAGGAGAGGACATGCCATCCTGTGGGGGATTTCCGCTGTAAAAATCACCACTGCA
TCCCTCTTCGTTGGCAGTGTGATGGGCAAAATGACTGTGGAGATAACTCAGATGAGGAAAACTGTGCTCC
CCGGGAGTGCACAGAGAGCGAGTTTCGATGTGTCAATCAGCAGTGCATTCCCTCGCGATGGATCTGTGAC
CATTACAACGACTGTGGGGACAACTCAGATGAACGGGACTGTGAGATGAGGACCTGCCATCCTGAATATT
TTCAGTGTACAAGTGGACATTGTGTACACAGTGAACTGAAATGCGATGGATCCGCTGACTGTTTGGATGC
GTCTGATGAAGCTGATTGTCCCACACGCTTTCCTGATGGTGCATACTGCCAGGCTACTATGTTCGAATGC
AAAAACCATGTTTGTATCCCGCCATATTGGAAATGTGATGGCGATGATGACTGTGGCGATGGTTCAGATG
AAGAACTTCACCTGTGCTTGGATGTTCCCTGTAATTCACCAAACCGTTTCCGGTGTGACAACAATCGCTG
CATTTATAGTCATGAGGTGTGCAATGGTGTGGATGACTGTGGAGATGGAACTGATGAGACAGAGGAGCAC
TGTAGAAAACCGACCCCTAAACCTTGTACAGAATATGAATATAAGTGTGGCAATGGGCATTGCATTCCAC
ATGACAATGTGTGTGATGATGCCGATGACTGTGGTGACTGGTCCGATGAACTGGGTTGCAATAAAGGAAA
AGAAAGAACATGTGCTGAAAATATATGCGAGCAAAATTGTACCCAATTAAATGAAGGAGGATTTATCTGC
TCCTGTACAGCTGGGTTCGAAACCAATGTTTTTGACAGAACCTCCTGTCTAGATATCAATGAATGTGAAC
AATTTGGGACTTGTCCCCAGCACTGCAGAAATACCAAAGGAAGTTATGAGTGTGTCTGTGCTGATGGCTT
CACGTCTATGAGTGACCGCCCTGGAAAACGATGTGCAGCTGAGGGTAGCTCTCCTTTGTTGCTACTGCCT
GACAATGTCCGAATTCGAAAATATAATCTCTCATCTGAGAGGTTCTCAGAGTATCTTCAAGATGAGGAAT
ATATCCAAGCTGTTGATTATGATTGGGATCCCAAGGACATAGGCCTCAGTGTTGTGTATTACACTGTGCG
AGGGGAGGGCTCTAGGTTTGGTGCTATCAAACGTGCCTACATCCCCAACTTTGAATCCGGCCGCAATAAT
CTTGTGCAGGAAGTTGACCTGAAACTGAAATACGTAATGCAGCCAGATGGAATAGCAGTGGACTGGGTTG
GAAGGCATATTTACTGGTCAGATGTCAAGAATAAACGCATTGAGGTGGCTAAACTTGATGGAAGGTACAG
AAAGTGGCTGATTTCCACTGACCTGGACCAACCAGCTGCTATTGCTGTGAATCCCAAACTAGGGCTTATG
TTCTGGACTGACTGGGGAAAGGAACCTAAAATCGAGTCTGCCTGGATGAATGGAGAGGACCGCAACATCC
TGGTTTTCGAGGACCTTGGTTGGCCAACTGGCCTTTCTATCGATTATTTGAACAATGACCGAATCTACTG
GAGTGACTTCAAGGAGGACGTTATTGAAACCATAAAATATGATGGGACTGATAGGAGAGTCATTGCAAAG
GAAGCAATGAACCCTTACAGCCTGGACATCTTTGAAGACCAGTTATACTGGATATCTAAGGAAAAGGGAG
AAGTATGGAAACAAAATAAATTTGGGCAAGGAAAGAAAGAGAAAACGCTGGTAGTGAACCCTTGGCTCAC
TCAAGTTCGAATCTTTCATCAACTCAGATACAATAAGTCAGTGCCCAACCTTTGCAAACAGATCTGCAGC
CACCTCTGCCTTCTGAGACCTGGAGGATACAGCTGTGCCTGTCCCCAAGGCTCCAGCTTTATAGAGGGGA
GCACCACTGAGTGTGATGCAGCCATCGAACTGCCTATCAACCTGCCCCCCCCATGCAGGTGCATGCACGG
AGGAAATTGCTATTTTGATGAGACTGACCTCCCCAAATGCAAGTGTCCTAGCGGCTACACCGGAAAATAT
TGTGAAATGGCGTTTTCAAAAGGCATCTCTCCAGGAACAACCGCAGTAGCTGTGCTGTTGACAATCCTCT
TGATCGTCGTAATTGGAGCTCTGGCAATTGCAGGATTCTTCCACTATAGAAGGACCGGCTCCCTTTTGCC
TGCTCTGCCCAAGCTGCCAAGCTTAAGCAGTCTCGTCAAGCCCTCTGAAAATGGGAATGGGGTGACCTTC
AGATCAGGGGCAGATCTTAACATGGATATTGGAGTGTCTGGTTTTGGACCTGAGACTGCTATTGACAGGT
CAATGGCAATGAGTGAAGACTTTGTCATGGAAATGGGGAAGCAGCCCATAATATTTGAAAACCCAATGTA
CTCAGCCAGAGACAGTGCTGTCAAAGTGGTTCAGCCAATCCAGGTGACTGTATCTGAAAATGTGGATAAT
AAGAATTATGGAAGTCCCATAAACCCTTCTGAGATAGTTCCAGAGACAAACCCAACTTCACCAGCTGCTG
ATGGAACTCAGGTGACAAAATGGAATCTCTTCAAACGAAAATCTAAACAAACTACCAACTTTGAAAATCC
AATCTATGCACAGATGGAGAACGAGCAAAAGGAAAGTGTTGCTGCGACACCACCTCCATCACCTTCGCTC
CCTGCTAAGCCTAAGCCTCCTTCGAGAAGAGACCCAACTCCAACCTATTCTGCAACAGAAGACACTTTTA
AAGACACCGCAAATCTTGTTAAAGAAGACTCTGAAGTATAGCTATACCAGCTATTTAGGGAATAATTAGA
AACACACTTTTGCACATATATTTITTACAAACAGATGAAAAAAGTTAACATTCAGTACTTTATGAAAAAA
ATATATTTTTCCCTGTTTGCCTATAGTTGGAGGTATCCTGTGTGTCTTTTTTTACTTATGCCGTCTCATA
TTTTTACAAATAATTATCACAATGTACTATATGTATATCTTTGCACTGAAGTTGTCTGAAGGTAATACTA
TAAATATATTGTATATTTGTAAATTTTGGAAAGATTATCCTGTTACTGAATTTGCTAATAAAGATGTCTG
CTGATTTGGTTGGTGATCATTATAGTAAATGATCCAACAAGAAAAGGAATTGACTGGGGACCTTTAGCCG
TGTCTAAAGAAGAGGCACCACTCATATTTCCTATAAAATTATCTAGGAAAGGAATCCAGGCCCCGCTCTT
GGGTCCATTTTTACACATTAGCACTTAATTAATGTTCAATATTACATGTCAATTTGATTAATGGCTATGT
TGATAGGGGCCACTATGTGTTGTATAGACATCTGGACTTGACTGTAGACTCCTCAGATAATACAGAAGGT
AGGAAAAGCAATTCAGTTTGGCCCTTCTGTGTGTTGGCATTGTCTAACCAGAACTCTCTGTTTCATGTGT
GTTCTCTCACTAGCTGCCAAGACAACATTTTTATTTGTGATGTCTATGAGGAAATCCCATATCATTAAGT
GCCAGTGTCCTGCATTGAGTTTGTGGTTAATTAAATGAGCTCTTCTGCTGATGGACCCTGGAGCAATTTC
TCCCCTCACCTGACATTCAAGGTGGTCACCTGCCCTAGTAGTTGGAGCTCAGTAGCTGAATTTCTGAAAC
CAAATCTGTGTCTTCATAAAATAAGGTGCAAAAAAAAAAAATACCAGTTAAGTAAAGCCTCAACTGGGTT
TTTGTTTCTATGAAAATATCATTATAATCACTATTTATTTCCTAAGTTGAACCTGAATAGAAAGGGAAAC
CATTCTTATTAAGCTTTTTATTAGGCCCTGTGGCTAAATGTGTACATTTATATTAGAATGTACTGTACAG
TCCAGATCTTTTCTTTAATTCTTATTGGTTTTTTTTTTTTTAGAGATGGAGTCTTGCTATATTGCCAAGG
CTGATCTTGAAGTCCTGGGCTCAAGTGATCCTCCCACCTCAGCCTCCTGAGTGGTTGGGGTTACGGGCGT
GAGCCACTGTGCCTGGCTTCCAGCTCTCCTCTTAAATAGTGGGTATAGTCTGCACAACAGGAACCATGGC
AGGAATATACACTTTCCCATAGCAAATAGCATACCTGACTCTCTGTGCTAATATTGCACATTTGTTAAAC
AATGAATGAATGGATGGATGGATGGATGGATGAATGAATGAAACATATACTACTGATTATTTTATTCCAG
AGTTCTCAAAATATTTGTTGCTGATATTTTGAGTGCTGACTGTAATTACTTTGATTAGATAAACAACTGG
AAATAATGCTGCTGAAAAAGTTCTAATAAATGTGTATTTTATCAGA.

One example of a protein sequence from the above LRP2 DNA is:

>sp|P98164|LRP2_HUMAN Low-density lipoprotein receptor-related
protein 2 OS = Homo sapiens OX = 9606 GN = LRP2 PE = 1 SV = 3
(SEQ ID NO. 940)
MDRGPAAVACTLLLALVACLAPASGQECDSAHFRCGSGHCIPADWRCDGTKDCSDDADEI
GCAVVTCQQGYFKCQSEGQCIPNSWVCDQDQDCDDGSDERQDCSQSTCSSHQITCSNGQC
IPSEYRCDHVRDCPDGADENDCQYPTCEQLTCDNGACYNTSQKCDWKVDCRDSSDEINCT
EICLHNEFSCGNGECIPRAYVCDHDNDCQDGSDEHACNYPTCGGYQFTCPSGRCIYQNWV
CDGEDDCKDNGDEDGCESGPHDVHKCSPREWSCPESGRCISIYKVCDGILDCPGREDENN
TSTGKYCSMTLCSALNCQYQCHETPYGGACFCPPGYIINHNDSRTCVEFDDCQIWGICDQ
KCESRPGRHLCHCEEGYILERGQYCKANDSFGEASIIFSNGRDLLIGDIHGRSFRILVES
QNRGVAVGVAFHYHLQRVFWTDTVQNKVFSVDINGLNIQEVLNVSVETPENLAVDWVNNK
IYLVETKVNRIDMVNLDGSYRVTLITENLGHPRGIAVDPTVGYLFFSDWESLSGEPKLER
AFMDGSNRKDLVKTKLGWPAGVTLDMISKRVYWVDSRFDYIETVTYDGIQRKTVVHGGSL
IPHPFGVSLFEGQVFFTDWTKMAVLKANKFTETNPQVYYQASLRPYGVTVYHSLRQPYAT
NPCKDNNGGCEQVCVLSHRTDNDGLGFRCKCTFGFQLDTDERHCIAVQNFLIFSSQVAIR
GIPFTLSTQEDVMVPVSGNPSFFVGIDFDAQDSTIFFSDMSKHMIFKQKIDGTGREILAA
NRVENVESLAFDWISKNLYWTDSHYKSISVMRLADKTRRTVVQYLNNPRSVVVHPFAGYL
FFTDWFRPAKIMRAWSDGSHLLPVINTTLGWPNGLAIDWAASRLYWVDAYFDKIEHSTFD
GLDRRRLGHIEQMTHPFGLAIFGEHLFFTDWRLGAIIRVRKADGGEMTVIRSGIAYILHL
KSYDVNIQTGSNACNQPTHPNGDCSHFCFPVPNFQRVCGCPYGMRLASNHLTCEGDPTNE
PPTEQCGLFSFPCKNGRCVPNYYLCDGVDDCHDNSDEQLCGTLNNTCSSSAFTCGHGECI
PAHWRCDKRNDCVDGSDEHNCPTHAPASCLDTQYTCDNHQCISKNWVCDTDNDCGDGSDE
KNCNSTETCQPSQFNCPNHRCIDLSFVCDGDKDCVDGSDEVGCVLNCTASQFKCASGDKC
IGVTNRCDGVFDCSDNSDEAGCPTRPPGMCHSDEFQCQEDGICIPNFWECDGHPDCLYGS
DEHNACVPKTCPSSYFHCDNGNCIHRAWLCDRDNDCGDMSDEKDCPTQPFRCPSWQWQCL
GHNICVNLSVVCDGIFDCPNGTDESPLCNGNSCSDFNGGCTHECVQEPFGAKCLCPLGFL
LANDSKTCEDIDECDILGSCSQHCYNMRGSFRCSCDTGYMLESDGRTCKVTASESLLLLV
ASQNKIIADSVTSQVHNIYSLVENGSYIVAVDFDSISGRIFWSDATQGKTWSAFQNGTDR
RVVFDSSIILTETIAIDWVGRNLYWTDYALETIEVSKIDGSHRTVLISKNLTNPRGLALD
PRMNEHLLFWSDWGHHPRIERASMDGSMRTVIVQDKIFWPCGLTIDYPNRLLYFMDSYLD
YMDFCDYNGHHRRQVIASDLIIRHPYALTLFEDSVYWTDRATRRVMRANKWHGGNQSVVM
YNIQWPLGIVAVHPSKQPNSVNPCAFSRCSHLCLLSSQGPHFYSCVCPSGWSLSPDLLNC
LRDDQPFLITVRQHIIFGISLNPEVKSNDAMVPIAGIQNGLDVEFDDAEQYIYWVENPGE
IHRVKTDGTNRTVFASISMVGPSMNLALDWISRNLYSTNPRTQSIEVLTLHGDIRYRKTL
IANDGTALGVGFPIGITVDPARGKLYWSDQGTDSGVPAKIASANMDGTSVKTLFTGNLEH
LECVTLDIEEQKLYWAVTGRGVIERGNVDGTDRMILVHQLSHPWGIAVHDSFLYYTDEQY
EVIERVDKATGANKIVLRDNVPNLRGLQVYHRRNAAESSNGCSNNMNACQQICLPVPGGL
FSCACATGFKLNPDNRSCSPYNSFIVVSMLSAIRGFSLELSDHSETMVPVAGQGRNALHV
DVDVSSGFIYWCDFSSSVASDNAIRRIKPDGSSLMNIVTHGIGENGVRGIAVDWVAGNLY
FTNAFVSETLIEVLRINTTYRRVLLKVTVDMPRHIVVDPKNRYLFWADYGQRPKIERSFL
DCTNRTVLVSEGIVTPRGLAVDRSDGYVYWVDDSLDIIARIRINGENSEVIRYGSRYPTP
YGITVFENSIIWVDRNLKKIFQASKEPENTEPPTVIRDNINWLRDVTIFDKQVQPRSPAE
VNNNPCLENNGGCSHLCFALPGLHTPKCDCAFGTLQSDGKNCAISTENFLIFALSNSLRS
LHLDPENHSPPFQTINVERTVMSLDYDSVSDRIYFTQNLASGVGQISYATLSSGIHTPTV
IASGIGTADGIAFDWITRRIYYSDYLNQMINSMAEDGSNRTVIARVPKPRAIVLDPCQGY
LYWADWDTHAKIERATLGGNFRVPIVNSSLVMPSGLTLDYEEDLLYWVDASLQRIERSTL
TGVDREVIVNAAVHAFGLTLYGQYIYWTDLYTQRIYRANKYDGSGQIAMTTNLLSQPRGI
NTVVKNQKQQCNNPCEQFNGGCSHICAPGPNGAECQCPHEGNWYLANNRKHCIVDNGERC
GASSFTCSNGRCISEEWKCDNDNDCGDGSDEMESVCALHTCSPTAFTCANGRCVQYSYRC
DYYNDCGDGSDEAGCLFRDCNATTEFMCNNRRCIPREFICNGVDNCHDNNTSDEKNCPDR
TCQSGYTKCHNSNICIPRVYLCDGDNDCGDNSDENPTYCTTHTCSSSEFQCASGRCIPQH
WYCDQETDCFDASDEPASCGHSERTCLADEFKCDGGRCIPSEWICDGDNDCGDMSDEDKR
HQCQNQNCSDSEFLCVNDRPPDRRCIPQSWVCDGDVDCTDGYDENQNCTRRTCSENEFTC
GYGLCIPKIFRCDRHNDCGDYSDERGCLYQTCQQNQFTCQNGRCISKTFVCDEDNDCGDG
SDELMHLCHTPEPTCPPHEFKCDNGRCIEMMKLCNHLDDCLDNSDEKGCGINECHDPSIS
GCDHNCTDTLTSFYCSCRPGYKLMSDKRTCVDIDECTEMPFVCSQKCENVIGSYICKCAP
GYLREPDGKTCRONSNIEPYLIFSNRYYLRNLTIDGYFYSLILEGLDNVVALDFDRVEKR
LYWIDTQRQVIERMFLNKTNKETIINHRLPAAESLAVDWVSRKLYWLDARLDGLFVSDLN
GGHRRMLAQHCVDANNTFCFDNPRGLALHPQYGYLYWADWGHRAYIGRVGMDGTNKSVII
STKLEWPNGITIDYTNDLLYWADAHLGYIEYSDLEGHHRHTVYDGALPHPFAITIFEDTI
YWTDWNTRTVEKGNKYDGSNRQTLVNTTHRPFDIHVYHPYRQPIVSNPCGTNNGGCSHLC
LIKPGGKGFTCECPDDFRTLQLSGSTYCMPMCSSTQFLCANNEKCIPIWWKCDGQKDCSD
GSDELALCPQRFCRLGQFQCSDGNCTSPQTLCNAHQNCPDGSDEDRLLCENHHCDSNEWQ
CANKRCIPESWQCDTFNDCEDNSDEDSSHCASRTCRPGQFRCANGRCIPQAWKCDVDNDC
GDHSDEPIEECMSSAHLCDNFTEFSCKTNYRCIPKWAVCNGVDDCRDNSDEQGCEERTCH
PVGDFRCKNHHCIPLRWQCDGQNDCGDNSDEENCAPRECTESEFRCVNQQCIPSRWICDH
YNDCGDNSDERDCEMRTCHPEYFQCTSGHCVHSELKCDGSADCLDASDEADCPTRFPDGA
YCQATMFECKNHVCIPPYWKCDGDDDCGDGSDEELHLCLDVPCNSPNRFRCDNNRCIYSH
EVCNGVDDCGDGTDETEEHCRKPTPKPCTEYEYKCGNGHCIPHDNVCDDADDCGDWSDEL
GCNKGKERTCAENICEQNCTQLNEGGFICSCTAGFETNVFDRTSCLDINECEQFGTCPQH
CRNTKGSYECVCADGFTSMSDRPGKRCAAEGSSPLLLLPDNVRIRKYNLSSERFSEYLQD
EEYIQAVDYDWDPKDIGLSVVYYTVRGEGSRFGAIKRAYIPNFESGRNNLVQEVDLKLKY
VMQPDGIAVDWVGRHIYWSDVKNKRIEVAKLDGRYRKWLISTDLDQPAAIAVNPKLGLMF
WTDWGKEPKIESAWMNGEDRNILVFEDLGWPTGLSIDYLNNDRIYWSDFKEDVIETIKYD
GTDRRVIAKEAMNPYSLDIFEDQLYWISKEKGEVWKONKFGQGKKEKTLVVNPWLTQVRI
FHQLRYNKSVPNLCKQICSHLCLLRPGGYSCACPQGSSFIEGSTTECDAAIELPINLPPP
CRCMHGGNCYFDETDLPKCKCPSGYTGKYCEMAFSKGISPGTTAVAVLLTILLIVVIGAL
AIAGFFHYRRTGSLLPALPKLPSLSSLVKPSENGNGVTFRSGADLNMDIGVSGFGPETAI
DRSMAMSEDFVMEMGKQPIIFENPMYSARDSAVKVVQPIQVTVSENVDNKNYGSPINPSE
IVPETNPTSPAADGTQVTKWNLFKRKSKQTTNFENPIYAQMENEQKESVAATPPPSPSLP
AKPKPPSRRDPTPTYSATEDTFKDTANLVKEDSEV.

As used herein, “target sequence” refers to a contiguous portion of the nucleotide sequence of an mRNA molecule formed during the transcription of a gene of interest for example a CD320 gene or an LRP2 gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

“G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine, 2′-deoxythymidine or thymidine. However, it will be understood that the term “ribonucleotide” or “nucleotide” or “deoxyribonucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

The term “siRNA” refers to a compound, cocktail, composition or agent that contains RNA as that term is defined herein, and which mediates the targeted cleavage of an RNA transcript via the RISC/AGO (RNA-induced silencing complex) complex, whereby the guide strand of the siRNA hybridizes with its complementary mRNA molecule. The mRNA is degraded by the RISC/AGO complex, which has RNAse cleave activity, resulting in mRNA degradation and the protein encoded by the mRNA is not produced or is produced at a reduced level as compared to untreated cell. This causes the “knockdown” effect or reduced protein levels of the gene targeted by the siRNA compared to control treated cells. The siRNA modulates, e.g., inhibits, the expression of CD320 in a cell or LRP2 in a cell, e.g., a cell within a subject, such as a mammalian subject.

In one embodiment, an RNAi agent of the invention includes a single stranded RNA that interacts with a target RNA sequence, e.g., a CD320 or LRP2 target mRNA sequence, to direct the cleavage of the target RNA. Without wishing to be bound by theory, it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair (bp) short interfering RNAs with characteristic two base 3′ overhangs (Bernstein, et al., (2001) Nature 409:363). Initially, the siRNAs may consist of two RNA strands, an antisense (or guide) strand and a sense (or passenger) strand, which form a duplex that varies in length from 10-80 bp in length with or without a 3′ nucleotide overhang. A dsRNA can include one or more single-stranded overhang(s) of one or more nucleotides. In one embodiment, at least one end of the dsRNA has a single-stranded nucleotide overhang of 1 to 4, generally 1 or 2 nucleotides. In another embodiment, the antisense strand of the dsRNA has 1-10 nucleotide overhangs each at the 3′ end and the 5′ end over the sense strand. In further embodiments, the sense strand of the dsRNA has 1-10 nucleotide overhangs each at the 3′ end and the 5′ end over the antisense strand.

The siRNA are then incorporated into an RNA-induced silencing complex (RISC) where one or more helicases unwind the siRNA duplex, enabling the complementary antisense (guide) strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., a CD320 or LRP2 gene. Accordingly, the term “siRNA” is also used herein to refer to an RNAi as described above.

In another embodiment, the RNAi agent may be a single-stranded siRNA that is introduced into a cell or organism to inhibit a target mRNA. Single-stranded RNAi agents bind to the RISC endonuclease Argonaute 2, which then cleaves the target mRNA. The single-stranded siRNAs are generally 15-80 nucleotides and may be chemically modified to improve metabolic stability and activity; wherein one or multiple pyrimidine nucleotides could be modified as 2′-deoxy-2′-fluoronucleotides, one or more purine nucleotides could be modified as 2′-deoxypurine nucleotides and, moreover, wherein terminal cap modifications could be present at the 3′ or 5′ ends; particularly by the introduction of one or more 2′-deoxythymidine nucleotides, or by the introduction of one or more phosphorothioate groups linking any nucleotides in the sequence but especially at the 3′ and 5′ end. In addition, a 3′-terminal phosphate or vinylphosphonate group could be introduced. Examples of such modifications would include but not be limited to modifications to the ribose moieties of the nucleotides such as: 2′-deoxy, 2′-deoxyfluoro, 2′-methoxy (2′-O-methyl) (Hutvanger et al., (2004) PLOS Biol 2, 0465-0475; Janas et al., (2019) Nuc Acid Res 47, 3306-3320; Jackson et al., (2006) RNA 12, 1197-1205), and 2′-methoxyethyl, wherein it is understood that the stereochemistry of the 2′-substituent could be in the ribo- or arabino-orientation. Another modification could be 2′-trifluoromethoxy. Other modifications to the ribose moieties could include bridging modifications such that the 2′-carbon of the sugar moiety is covalently linked to the 4′-carbon of the sugar moiety by a methylene or methoxymethylene group to afford bridged nucleotides described in the art as LNA and (S)-cET, respectively (Corey et al., (2018) Nuc Acid Res 46; 1584-1600). In addition, the sugar moiety could be modified by removal of the bond between carbons C2′ and C3′ to afford “open” chain nucleotides analogous to those described in WO 2011/139843 A2. The ribose moiety of the RNA nucleotides could also be replaced by a morpholino group to afford PMO nucleotides. Modifications to the phosphate diester moieties of the nucleotides are also possible and could include but not be limited to replacement of the phosphodiester group by phosphorothioate and thio-phosphoramidate (Eckstein et al., (2014) Nuc Acid Therapeutics 24, 374-387). The ends of the strand could be modified with 2′-deoxynucleotides such as dT and, further, the dT nucleotides could be modified by phosphorothioate groups in place of diphosphate esters. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150; 883-894.

In another embodiment, an “RNAi” for use in the compositions, uses, and methods of the invention is a double-stranded RNA and is referred to herein as a “double stranded RNAi agent,” “double-stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” (passenger) and “antisense” (guide) orientations with respect to a target RNA, i.e., a CD320 gene or LRP2 gene. In some embodiments of the invention, a double-stranded RNA (dsRNA) triggers the degradation of a target RNA, e.g., an mRNA, through a post-transcriptional gene-silencing mechanism referred to herein as RNA interference or RNAi.

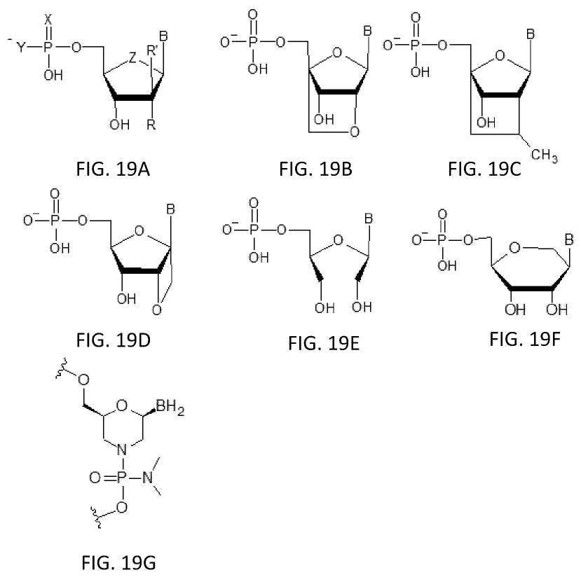

In general, the majority of nucleotides of each strand of a dsRNA molecule are ribonucleotides, but as described in detail herein, each or both strands can also include one or more non-ribonucleotides, e.g., a deoxyribonucleotide and/or a modified nucleotide. In addition, as used in this specification, an “RNAi agent” may include ribonucleotides with chemical modifications (Corey et al., (2018) Nuc Acid Res 46; 1584-1600); an RNAi agent may include substantial modifications at multiple nucleotides or at a single nucleotide. Such modifications may include all types of modifications disclosed herein or known in the art. Any such modifications, as used in a siRNA type molecule, are encompassed by “RNAi agent” for the purposes of this specification and claims. Examples of such modifications would include but not be limited to modifications to the ribose moieties of the nucleotides such as: 2′-deoxy, 2′-deoxyfluoro, 2′-methoxy (2′-O-methyl) (Hutvanger et al., (2004) PLOS Biol 2, 0465-0475; Janas et al., (2019) Nuc Acid Res 47, 3306-3320; Jackson et al., (2006) RNA 12, 1197-1205), and 2′-methoxyethyl, wherein it is understood that the stereochemistry of the 2′-substituent could be in the ribo- or arabino-orientation. Another modification could be 2′-trifluoromethoxy. Other modifications to the ribose moieties could include bridging modifications such that the 2′-carbon of the sugar moiety is covalently linked to the 4′-carbon of the sugar moiety by a methylene or methoxymethylene group to afford bridged nucleotides described in the art as LNA and (S)-cET, respectively (Corey et al., (2018) Nuc Acid Res 46; 1584-1600). In addition, the sugar moiety could be modified by removal of the bond between carbons C2′ and C3′ to afford “open” chain nucleotides analogous to those described in WO 2011/139843 A2. The ribose moiety of the RNA nucleotides could also be replaced by a morpholino group to afford PMO nucleotides. Modifications to the phosphate diester moieties of the nucleotides are also possible and could include but not be limited to replacement of the phosphodiester group by phosphorothioate and thio-phosphoramidate (Eckstein et al., (2014) Nuc Acid Therapeutics 24, 374-387). The ends of the sense and antisense strands could be modified with 2′-deoxynucleotides such as dT and, further, the dT nucleotides could be modified by phosphorothioate groups in place of diphosphate esters (FIG. 19).

Chemical modifications to the ribonucleotides could be made at any individual or combination of nucleotides in the antisense and sense strands. In some cases, all the nucleotides in either the antisense or sense strand, or in both the antisense and sense strands are chemically modified (Allerson et al., (2005) J Med Chem 48, 901-904). In other cases, only some of the nucleotides in the antisense or sense strand, or in both the antisense and sense strands are chemically modified (Chiu et al., (2003) RNA 9, 1034-1048). In yet other cases, the modifications could follow a pattern of alternating 2′-methoxy and 2′-fluoro modifications to either or both strands of the siRNA and sometimes the complementary nucleotides of the antisense and sense strands could contain chemical modifications which are not identical, for example, where one member of a complementary nucleotide pair has a 2′-methoxy modification and the other member has a 2′-fluoro modification (Choung et al. (2006) Biochem Biophys Res Commun 342, 919-927; Hassler et al., (2018) Nucleic Acid Res 46, 2185-2196).

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi agent may comprise one or more nucleotide overhangs.

In one embodiment, an RNAi agent of the invention is a dsRNA of 20-30 nucleotides that interacts with a target RNA sequence, e.g., a CD320 target mRNA sequence or a LRP2 target mRNA sequence, to direct the cleavage of the target RNA.

The term “antisense strand” refers to the strand of a double stranded RNAi agent which includes a region that is substantially complementary to a target sequence (e.g., a human CD320 mRNA or a LRP2 mRNA). As used herein, the term “region complementary to part of an mRNA encoding CD320 or LRP2” refers to a region on the antisense strand that is substantially complementary to part of a mRNA sequence that codes for either CD320 or LRP2. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus. For example, substantially complementary can in certain embodiments mean that in a hybridized pair of nucleobase sequences, at least 85% but not all of the bases in a contiguous sequence of a first polynucleotide will hybridize with the same number of bases in a contiguous sequence of a second polynucleotide.

The term “sense strand,” as used herein, refers to the strand of a dsRNA that includes a region that is substantially complementary to a region of the antisense strand.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. For example, a complementary sequence is sufficient to allow the relevant function of the nucleic acid to proceed, e.g., RNAi. The skilled person will be able to determine the set of conditions most appropriate for a test of complementarity of two sequences in accordance with the ultimate application of the hybridized nucleotides.

Sequences can be “fully complementary” with respect to each when there is base-pairing of the nucleotides of the first nucleotide sequence with the nucleotides of the second nucleotide sequence over the entire length of the first and second nucleotide sequences. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs include, but are not limited to, G: U Wobble or Hoogstein base pairing.

The terms “complementary,” “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding CD320 or an mRNA encoding LRP2) including a 5′ UTR, an open reading frame (ORF), or a 3′ UTR. For example, a polynucleotide is complementary to at least a part of a CD320 mRNA or LRP2 mRNA if the sequence is substantially complementary to a non-interrupted portion of an mRNA encoding CD320 or LRP2.

The term “inhibiting,” as used herein, is used interchangeably with “reducing,” “silencing,” “downregulating,” “suppressing” and other similar terms, and includes any level of inhibition.

The phrase “inhibiting expression of a CD320,” “inhibiting expression of a LRP2” as used herein, includes inhibition of expression of any CD320 or LRP2 gene (such as the identified gene from, e.g., a mouse, a rat, a monkey, or a human) as well as variants, (e.g., naturally occurring variants), or mutants of the identified gene. Thus, the CD320 or LRP2 gene may be a wild-type CD320 or LRP2 gene, a mutant CD320 or LRP2 gene, or a transgenic CD320 or LRP2 gene in the context of a genetically manipulated cell, group of cells, or organism.

“Inhibiting expression of a CD320 gene” or “Inhibiting expression of a LRP2 gene” includes any level of inhibition of a CD320 gene or a LRP2 gene, e.g., at least partial suppression of the expression of a CD320 or LRP2 gene, such as an inhibition of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In a preferred embodiment the inhibition is assessed by expressing the level of CD320 or LRP2 protein in treated cells as a percentage of the level of mRNA in control cells, using the following formula:
Normalized protein level for treated cells/Normalized protein level for control cells. The control cells are the negative control siRNA. Normalized means the protein level is normalized to the level of a housekeeping protein.

The expression of a CD320 or LRP2 gene may be assessed based on the level of any variable associated with CD320 or LRP2 gene expression, e.g., CD320 or LRP2 mRNA level, CD320 or LRP2 protein level. Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

Contacting a cell with a RNAi agent, either ds or ss as used herein, includes contacting a cell by any possible means whether in vivo or in vitro. Contacting a cell with a RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

A “patient” or “subject,” as used herein, is intended to include either a human or non-human animal, preferably a mammal, e.g., a monkey. Most preferably, the subject or patient is a human.

A “CD320-associated disease,” as used herein, is intended to include any disease associated with a perturbation of the CD320 gene, or protein, polymorphisms, single nucleotide polymorphisms (SNPs) as well as epigenetic modifications of the CD320 gene. Such a disease may be caused, for example, by excess production of the CD320 protein, by CD320 gene mutations, by abnormal cleavage of the CD320 protein, by abnormal folding of the CD320 protein, by abnormal interactions between CD320 itself or with other proteins or other endogenous or exogenous substances. For example, cancer may be a CD320-associated disease. The degree of inhibition of protein expression may be measured by western blotting.

A “LRP2-associated disease,” as used herein, is intended to include any disease associated with a perturbation of the LRP2 gene, protein, polymorphisms, SNPs as well as epigenetic modifications of the CD320 gene. Such a disease may be caused, for example, by excess production of the LRP2 protein, by LRP2 gene mutations, by abnormal cleavage of the LRP2 protein, by abnormal folding of the LRP2 protein, by abnormal interactions between LRP2 molecules and other proteins or other endogenous or exogenous substances. For example, cancer may be a LRP2-associated disease. The degree of inhibition of protein expression may be measured by western blotting.

“Therapeutically effective amount,” as used herein, is intended to include the amount of an RNAi agent that, when administered to a cell or a patient for treating a CD320 associated disease or a LRP2 associated disease, is sufficient to effect treatment of the disease (e.g., by diminishing, ameliorating or maintaining the existing disease or one or more symptoms of disease or by preferentially causing the death of a disease cell as compared to a non-disease cell). The “therapeutically effective amount” may vary depending on the RNAi agent, how the agent is administered, the disease and its severity and the history, age, weight, family history, genetic makeup, stage of pathological processes mediated by CD320 or LRP2 expression, the types of preceding or concomitant treatments, if any, and other individual characteristics of the patient to be treated.

“Prophylactically effective amount,” as used herein, is intended to include the amount of an RNAi agent that, when administered to a subject who does not yet experience or display symptoms of a CD320 associated disease or a LRP2 associated disease, but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired local or systemic effect at a reasonable benefit/risk ratio applicable to any treatment. RNAi agents employed in the methods of the present invention may be administered in a sufficient amount to produce a reasonable benefit/risk ratio applicable to such treatment.

### Pharmaceutical Compositions

The methods described herein include administration of a LRP2 inhibiting composition and/or a CD320 inhibiting composition, e.g., a first siRNA targeting a CD320 gene and/or a second siRNA targeting a LRP2 gene. In some embodiments, the LRP2 inhibiting composition and/or the CD320 inhibiting composition is a pharmaceutical composition.

The methods described herein also include administration of one or multiple LRP2 inhibiting compositions and/or one or multiple CD320 inhibiting compositions, e.g., one or more siRNAs targeting a CD320 gene and/or one or more siRNAs targeting an LRP2 gene. It is understood that such compositions could be chemically modified in a variety of ways and that such modifications need not be identical in compositional mixtures. In some embodiments, the LRP2 inhibiting composition and/or the CD320 inhibiting composition is a pharmaceutical composition.

The pharmaceutical compositions of the present invention may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; or intracranial, e.g., intraparenchymal, intrathecal or intraventricular, administration.

The compositions can be delivered in a manner to target a particular tissue, such as the lung cells or breast cells or brain cells or bladder cells or uterine cells or cervix cells or prostate cells. Pharmaceutical compositions can be delivered by injection directly into the brain. The injection can be by stereotactic injection into a particular region of the brain (e.g., the substantia nigra, cortex, hippocampus, striatum, or globus pallidus), or the dsRNA can be delivered into multiple regions of the central nervous system (e.g., into multiple regions of the brain, and/or into the spinal cord). The dsRNA can also be delivered into diffuse regions of the brain (e.g., diffuse delivery to the cortex of the brain). In general siRNAs are administered 1) by intratumoral injection, 2) by systemic injection, 3) by slow release from an implanted polymer. Other tissue specificity could be achieved by antibody or small molecule conjugation, or by a tissue-specific delivery device (e.g., a catheter can be used to deliver to the bladder).

In one embodiment, an RNAi targeting either LRP2 or the CD320 can be delivered by way of a cannula or other delivery device having one end implanted in a tissue. The cannula can be connected to a reservoir of the RNAi composition. The flow or delivery can be mediated by a pump, e.g., an osmotic pump or minipump. In one embodiment, a pump and reservoir are implanted in an area distant from the tissue, e.g., in the abdomen, and delivery is affected by a conduit leading from the pump or reservoir to the site of release.

Accordingly, in some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described RNAi agents and one or more pharmaceutically acceptable excipients. Pharmaceutically acceptable excipients (excipients) are substances other than the Active Pharmaceutical Ingredient (API, therapeutic product, e.g., CD320 RNAi agent or LRP2 RNAi agent) that are intentionally included in the drug delivery system. Excipients do not exert or are not intended to exert a therapeutic effect at the intended dosage. Excipients can act to a) aid in processing of the drug delivery system during manufacture, b) protect, support, or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor® ELTM (BASF, Parsippany, N.J.) or phosphate buffered saline (PBS). The composition, understood to include formulations and drug delivery systems, should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

### Dosage and Timing

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the LRP2 inhibiting composition and or the CD320-inhibiting compositions encompassed by the invention can be made using conventional methodologies or on the basis of in vivo testing using an appropriate animal model, as described elsewhere herein.

In general, a suitable dose of a pharmaceutical composition of the LRP2 inhibiting composition and/or the CD320-inhibiting composition will be in the range of 0.01 to 300.0 milligrams per kilogram body weight of the recipient per day, generally in the range of 1 to 50 mg per kilogram body weight per day.

For example, the LRP2 inhibiting composition and/or the CD320-inhibiting composition can be an siRNA composition of one or more siRNAs, and can be administered at, 0.01 mg/kg, 0.05 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.628 mg/kg, 2 mg/kg, 3 mg/kg, 5.0 mg/kg, 10 mg/kg, 20 mg/kg, 30 mg/kg, 40 mg/kg, 50 mg/kg, 100 mg/kg, 200 mg/kg, 400 mg/kg per single dose. In another embodiment, the dosage is between 0.15 mg/kg and 0.3 mg/kg. For example, the LRP2 and/or the CD320-inhibiting composition can be administered at a dose of 0.15 mg/kg, 0.2 mg/kg, 0.25 mg/kg, or 0.3 mg/kg. In an embodiment, the LRP2 and/or the CD320-inhibiting composition is administered at a dose of 0.3 mg/kg.

The pharmaceutical composition may be administered once daily, or once or twice every 5, 10, 15, 20, 21, 22, 23, 24, 25, 26, 27, 28, 29, or 30 days. The dosage unit can be compounded for delivery over several days, e.g., using a conventional sustained release formulation which provides sustained release of the LRP2 inhibiting composition and/or the CD320-inhibiting composition over a several day period. Sustained release formulations are well known in the art and are particularly useful for delivery of agents at a particular site, such as could be used with the agents of the present invention.

In an embodiment, the LRP2-inhibiting composition and/or the CD320-inhibiting composition is dependent upon the tumor cell line, and the dosage is 0.3 mg/kg, and wherein the dose is administered once every 21 days. In another embodiment, the effective amount is 0.3 mg/kg and the effective amount is administered once every 21 days via a 70 minute infusion of 1 mL/min for 15 minutes followed by 3 mL/min for 55 minutes. In another embodiment, the effective amount is 0.3 mg/kg and the effective amount is administered at two doses every 21-28 days via a 60 minute infusion of 3.3 mL/min, or via a 70 minute infusion of 1.1 mL/min for 15 minutes followed by 3.3 mL/min for 55 minutes

A dosage of a LRP2-inhibiting composition and/or the CD320-inhibiting composition can be adjusted for treatment

A LRP2-inhibiting composition and/or the CD320-inhibiting composition can be administered in combination with other known agents effective in treatment of pathological processes mediated by target gene expression.

In another embodiment, the pharmaceutical composition is formulated for administration according to a dosage regimen described herein, e.g., not more than once every four weeks, not more than once every three weeks, not more than once every two weeks, or not more than once every week. In another embodiment, the administration of the pharmaceutical composition can be maintained for a month or longer, e.g., one, two, three, or six months, or one year or longer.

In embodiments of the pharmaceutical compositions described herein, the RNAi (e.g., dsRNA) is administered with a buffer solution. In embodiments, the buffer solution comprises acetate, citrate, prolamine, carbonate, or phosphate or any combination thereof. In embodiments, the buffer solution is phosphate buffered saline (PBS).

In embodiments of the pharmaceutical compositions described herein, the composition is administered intravenously.

In embodiments of the pharmaceutical compositions described herein, the composition is administered subcutaneously.

In certain embodiments, a pharmaceutical composition, e.g., a composition described herein, includes a lipid formulation. In embodiments, the composition is administered intravenously.

In some embodiments, a pharmaceutical composition, e.g., a composition described herein, includes a cationic polyamine formulation or nanoparticle (e.g., JetPEI). In some embodiments, the composition is administered intravenously.

In another embodiment, the pharmaceutical composition is formulated for administration according to a dosage regimen described herein, e.g., not more than once every four weeks, not more than once every three weeks, not more than once every two weeks, or not more than once every week. In another embodiment, the administration of the pharmaceutical composition can be maintained for a month or longer, e.g., one, two, three, or six months, or one year or longer.

In another embodiment, a composition containing an RNAi agent featured in the invention, e.g., a dsRNA targeting LRP2 or CD320, is administered with a non-RNAi therapeutic agent, such as an agent known to treat a cancer such as lung cancer. In another embodiment, a composition containing an RNAi agent featured in the invention, e.g., a dsRNA targeting LRP2 and/or CD320, is administered along with a non-RNAi therapeutic regimen, such as radiation, chemotherapy, immunotherapy, photodynamic therapy or a combination thereof.

In an aspect provided herein is a method of inhibiting LRP2 and/or CD320 expression in a cell, the method comprising: (a) introducing into the cell an RNAi agent (e.g., a dsRNA) described herein and (b) maintaining the cell of step (a) for a time sufficient to obtain degradation of the mRNA transcript of an LRP2 gene and/or CD320 gene, thereby inhibiting expression of the LRP2 gene and/or CD320 gene in the cell.

In an aspect provided herein is a method for reducing or inhibiting the expression of LRP2 gene and/or CD320 genes in a cell. The method includes: (a) introducing into the cell one or more complimentary double-stranded ribonucleic acid (dsRNA) molecules, in which one sequence is designated the sense strand and the other sequence the anti-sense strand, and wherein the anti-sense strand has significant complementarity to a portion of mRNA encoding for LRP2 or CD320. The complimentary region is 15-30 nucleotides in length, and generally 19-24 nucleotides in length, and the dsRNA, upon entering a cell expressing LRP2 and/or CD320, inhibits the expression of the LRP2 protein and/or CD320 protein by at least 10%, e.g., at least 20%, at least 30%, at least 40% or more; (b) single or repeated treatment of the cell with dsRNAs, as described in part (a), so as to maintain the inhibition of LRP2 and/or CD320 protein expression over a desired period of time by at least 10%, e.g., at least 20%, at least 30%, at least 40% or more.

In embodiments of the foregoing methods of inhibiting LRP2 and/or CD320 expression in a cell, the cell is treated ex vivo, in vitro, or in vivo. In embodiments, the cell is a melanoma, glioblastoma, lung carcinoma, triple negative breast carcinoma, renal carcinoma, pancreatic carcinoma, hepatocellular carcinoma, ovarian carcinoma and prostate carcinoma.

In some embodiments, the cell is present in a subject in need of treatment, prevention and/or management of a CD320-associated disease or a LRP2-associated disease.

In embodiments, the expression of LRP2 and/or CD320 is inhibited by at least 30%.

In embodiments, the RNAi (e.g., dsRNA) has an IC₅₀ in the range of 0.01-50 nM.

In embodiments, the RNAi (e.g., dsRNA) has an IC₅₀ in the range of 0.01-1 nM.

In certain embodiments, the cell is a mammalian cell (e.g., a human, non-human primate, or rodent cell).

In one embodiment, the cell is treated ex vivo, in vitro, or in vivo (e.g., the cell is present in a subject (e.g., a patient in need of treatment, prevention and/or management of a disorder related to LRP2 and/or CD320 expression).

In one embodiment, the subject is a mammal (e.g., a human) at risk, or diagnosed with a proliferation disorder.

In embodiments, the RNAi (e.g., dsRNA) is formulated as an lipid nanoparticle (LNP) polyplex (polyamine) formulation.

In embodiments, RNAi (e.g., dsRNA) is administered at a dose of 0.05001-500.01 mg/kg.

In embodiments, the RNAi (e.g., dsRNA) is administered at a concentration of 0.01 mg/kg-50.1 mg/kg bodyweight of the subject.

In embodiments, the RNAi (e.g., dsRNA) is formulated as an LNP formulation and is administered at a dose of 0.050.1-50.5 mg/kg.

In embodiments, the RNAi (e.g., dsRNA) has an IC₅₀ in the range of 0.01-10 nM.

In embodiments, the RNAi (e.g., dsRNA) or composition comprising the RNAi is administered according to a dosing regimen. In embodiments, the RNAi (e.g., dsRNA) or composition comprising the RNAi is administered as a single dose or at multiple doses, e.g., according to a dosing regimen.

The term “sample,” as used herein, includes a collection of fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from a tumor. In preferred embodiments, a “sample derived from a subject” refers to blood or plasma drawn from the subject. In further embodiments, a “sample derived from a subject” refers to tissue biopsy derived from the subject.

In one embodiment, an RNAi (e.g., a dsRNA) featured herein includes a first sequence of a dsRNA that is selected from the group consisting of the sense sequences of Table 1 and a second sequence that is selected from the group consisting of the corresponding antisense sequences of Table 1. It is understood that the suffix A (e.g., OSC17A) represents the antisense strand whereas the suffix S (e.g., OSC17S) represents the sense strand. In those instances when we refer to an siRNA with no suffix (e.g., OSC17), we mean that to indicate the dsRNA comprised of the antisense and sense strands corresponding to that number (e.g., OSC17A paired with OSC17S).

In some embodiments the RNAi is from about 15 to about 25 nucleotides in length, and in other embodiments the RNAi is from about 25 to about 30 nucleotides in length. An RNAi targeting CD320, upon contact with a cell expressing CD320, inhibits the expression of a CD320 gene by at least 10%, at least 20%, at least 25%, at least 30%, at least 35% or at least 40% or more, such as when assayed by a method as described herein. In one embodiment, the RNAi targeting CD320 is formulated in a stable nucleic acid lipid particle (SNALP).

In some embodiments the RNAi is from about 15 to about 25 nucleotides in length, and in other embodiments the RNAi is from about 25 to about 30 nucleotides in length. An RNAi targeting LRP2, upon contact with a cell expressing LRP2, inhibits the expression of a LRP2 gene by at least 10%, at least 20%, at least 25%, at least 30%, at least 35% or at least 40% or more, such as when assayed by a method as described herein. In one embodiment, the RNAi targeting LRP2 is formulated in a stable nucleic acid lipid particle (SNALP).

In some embodiments the RNAi is from about 15 to about 25 nucleotides in length, and in other embodiments the RNAi is from about 25 to about 30 nucleotides in length. An RNAi targeting CD320, upon contact with a cell expressing CD320, inhibits the expression of a CD320 gene by at least 10%, at least 20%, at least 25%, at least 30%, at least 35% or at least 40% or more, such as when assayed by a method as described herein. In one embodiment, the RNAi targeting CD320 is formulated as a complex, which may exist as a nanoparticle, with a cationic polyamine.

In some embodiments the RNAi is from about 15 to about 25 nucleotides in length, and in other embodiments the RNAi is from about 25 to about 30 nucleotides in length. An RNAi targeting LRP2, upon contact with a cell expressing LRP2, inhibits the expression of a LRP2 gene by at least 10%, at least 20%, at least 25%, at least 30%, at least 35% or at least 40% or more, such as when assayed by a method as described herein. In one embodiment, the RNAi targeting LRP2 is formulated as a complex, which may exist as a nanoparticle, with a cationic polyamine.

Referring now to Table 1-DNA sequences are illustrated, which are subsequently transcribed into shRNA, which hence targets the CD320 or LRP2 mRNA for destruction in the cell. shRNA sequences used in lentiviral vectors illustrates the sequences that were used to target the CD320 sequence coding for the CD320 protein and the LRP2 sequence coding for the LRP2 protein. The Each vector that carried a shRNA coding sequence also contained a unique drug resistance gene which would allow for selecting those cells that had taken up the shRNA as those cells that had not taken up the shRNA having the unique drug resistance gene would not survive. On day 2, drug selection was started. On day 3, the cells were harvested and plated in a new dish. Only the cells with a drug resistance gene, i.e., those cells that had taken up shRNA virus particles would survive this re-plating procedure. From day 4 on, each culture was closely observed for cell growth. The cells that were infected with the irrelevant control shRNA kept on growing as expected (since the shRNA was essentially a non-functional shRNA)—data not shown. The results for the cell lines that took up the CD320+LRP2 shRNAs are shown in Table 1.

TABLE 1

| | | | Anti-Sense | Location |
|---|---|---|---|---|
| Name | Target | Sense Sequence | Sequence | in DNA |
| shScramble | non-targeting | CCTAAGGTTAAGTC | CGAGGGCGACTTAAC | N/A |
| | control | GCCCTCG | CTTAGG | |
| | | (SEQ ID NO. 941) | (SEQ ID NO. 942) | |
| shCD320-#27 | CD320 | CCCTCAGAGACCTG | AAGAGCTCAGGTCTC | 1006- |
| | (NM_016579.3) | AGCTCTT | TGAGGG | 1026 |
| | | (SEQ ID NO. 943) | (SEQ ID NO. 944) | |
| shLRP2-#89 | LRP2 | CCTGTAATAAACAC | AAGAGTAGTGTTTAT | 2800- |
| | (NM_004525.2) | TACTCTT | TACAGG | 2820 |
| | | (SEQ ID NO. 945) | (SEQ ID NO. 946) | |

The preliminary studies show that cancer cells are selectively killed by CD320 and LRP2 knockdown, while normal cells remain unaffected (Table 2).

Table 2 shows the effect of simultaneous knockdown of CD320 and LRP2 on cell viability.

TABLE 2

| | | Outcome of CD320/LRP2 | |
|---|---|---|---|
| | | knockdown | |
| | | Cell arrest/death | Alive |
| Cancer (Lung) | HCC15 | √ | |
| | H157 | √ | |
| | H358 | √ | |
| | H1999 | √ | |
| Non-cancer | Normal fibroblast | | √ |
| | LDLR mutant fibroblast | | √ |

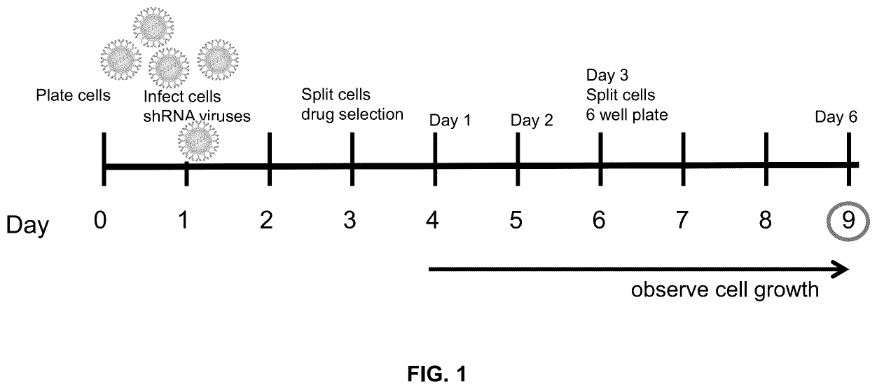

Additional cancer cell lines were also treated with the compounds described herein to determine whether cancer cell lines were more susceptible to growth inhibition and toxicity as compared to non-cancer cells of the same origin. Cell lines from skin, prostate, and brain cancers were screened similarly to the experimental outline in FIG. 1. Table 3 summarizes the effects of simultaneous knockdown of CD320 and LRP2 in cancer and normal cells.

TABLE 3

| | | | | shCD320 + | |
|---|---|---|---|---|---|
| Cell | shSCR | shCD320 | shLRP2 | shLRP2 | Comments |
| Normal cells | | | | | |
| GM05659 | +++ | +++ | +++ | +++ | no effect of knockdown |
| GM00701 | +++ | ++ | +++ | +++ | cells grow very slow; hard to determine |
| | | | | | if any affect of knockdown |
| SAEC | pending | | | | |
| Lung cells | | | | | |
| HCC15 | +++ | + | + | + | cells strongly affected by knockdown |
| H157 | +++ | + | ++ | + | senescent phenotype |
| H358 | +++ | ++ | ++ | ++ | morphology changes; cells rounded |
| H1993 | +++ | ++ | ++ | + | cells rounded; morphology change |
| Melanoma Cells | | | | | |
| MDA-MB-4353 | +++ | ++ | + | + | morphology change; cells strongly affected |
| | | | | | by double knockdown |
| Prostate cells | | | | | |
| LncAP | +++ | ++ | ++ | + | cells rounded; morphology change |
| PC3 | +++ | +++ | ++ | +++ | cells minimally to not affected by knockdown |
| DU-145 | +++ | ++ | + | ++ | cells modestly affected by knockdown |
| Glioblastoma | | | | | |
| A172 | +++ | + | + | 0 | cells strongly affected by knockdown |
| U251MG | +++ | + | ++ | 0 | cells strongly affected by knockdown |
| U343 | +++ | +++ | ++ | +++ | cells modestly affected by LRP2 knockdown |
| T98G | +++ | +++ | ++ | ++ | cells slightly affected by knockdown |

+++ cells unaffected compared to shSCR (control)
++ cells modestly affected compared to shSCR (control)
+ cells significantly affected compared to shSCR (control)
0 vast majority of cells killed compared to shSCR (control)

The screening results showed that lung, prostate, skin, and brain cancer cell lines were growth-inhibited or killed by the simultaneous knockdown (“double knockdown”) of CD320 and LRP2, while non-cancerous cells were unaffected.

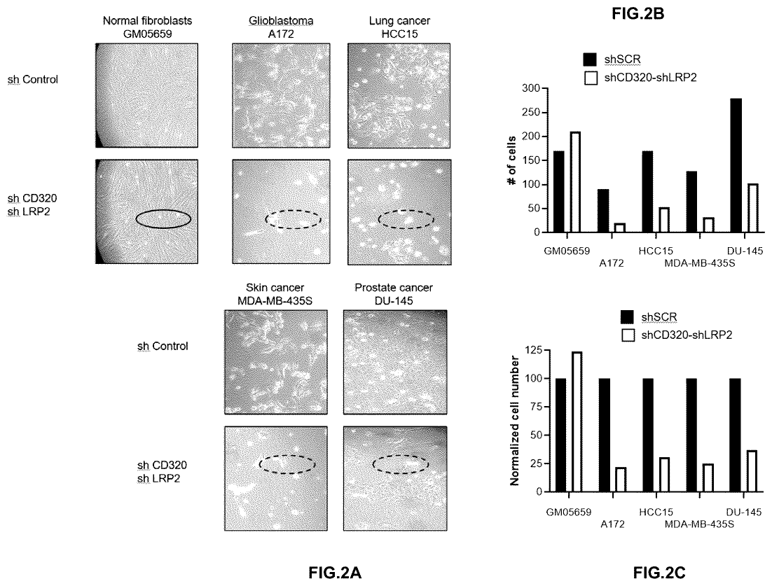

Referring now to FIG. 2, representative pictures of the cells were taken to record their phenotypes after the double knockdown of CD320 and LRP2 and to illustrate the sensitivity of cancer cell lines to knockdown of the expression of CD320 and LRP2 proteins.

Normal cells (GM05659 fibroblasts) or cancer cells were infected with lentiviruses expressing shRNAs to control sequences or to shCD320 and shLRP2 as described in FIG. 1. The cells were grown as described in FIG. 1. On the ninth day after transfection with the lentiviruses, pictures of the cells were taken. The solid line ovals indicate healthy growth of normal fibroblast infected with shRNAs to CD320 and LRP2. The broken line ovals indicate unhealthy dying cells of cancer cell lines infected with shRNAs to CD320 and LRP2.

These results support use of the compounds as therapeutics based upon decreasing expression of CD320 and LRP2 protein preferentially resulting in detrimental effects in cancer cells as compared to non-cancer cells. The original experiments were conducted using shRNAs delivered by lentiviral vectors. Short inhibitory RNAs (siRNAs), having a sequence complimentary to a portion of the CD320 protein and/or the LRP2 protein were designed. The siRNAs can be chemically modified to increase their stability and potency and reduce their immunogenicity, and multiple platforms exist for their delivery in clinical applications.

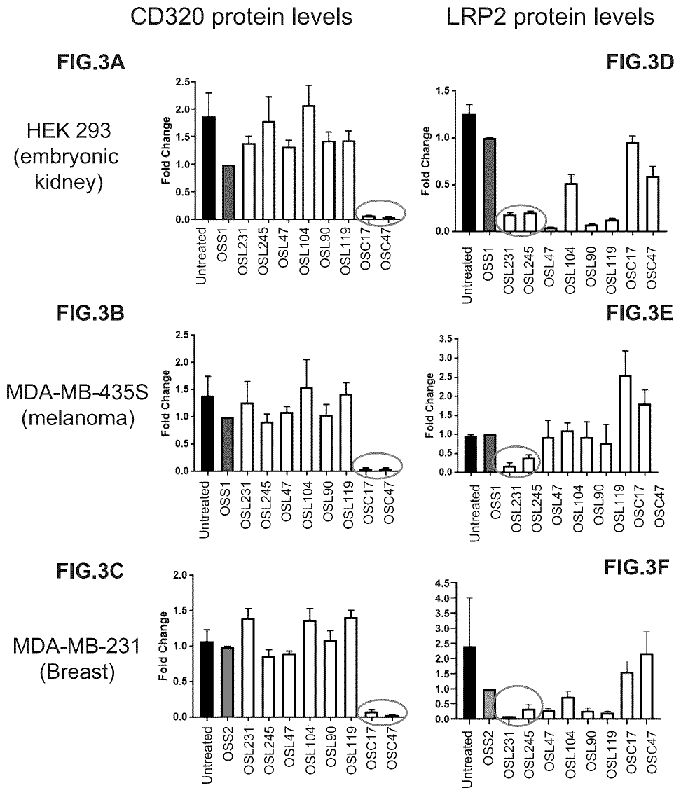

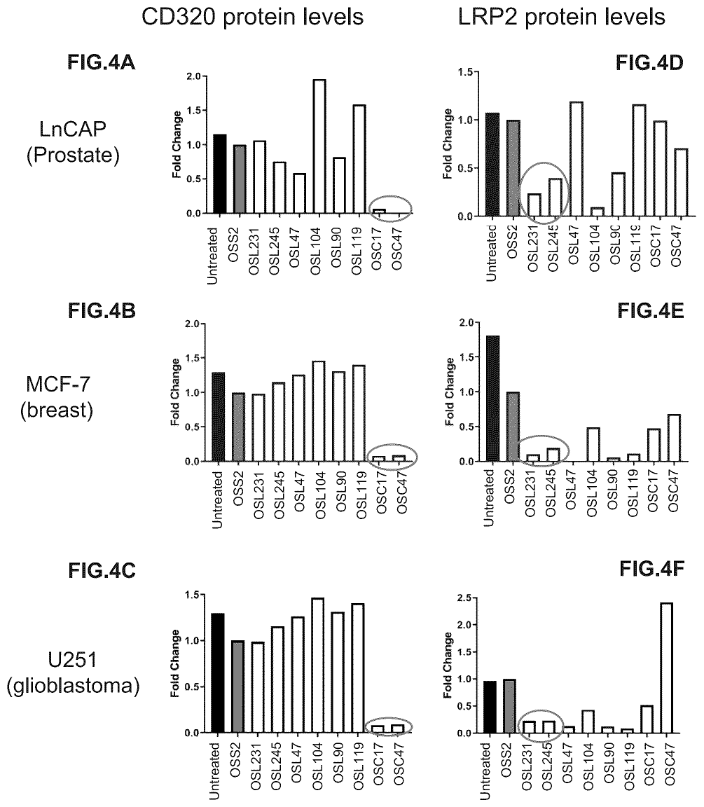

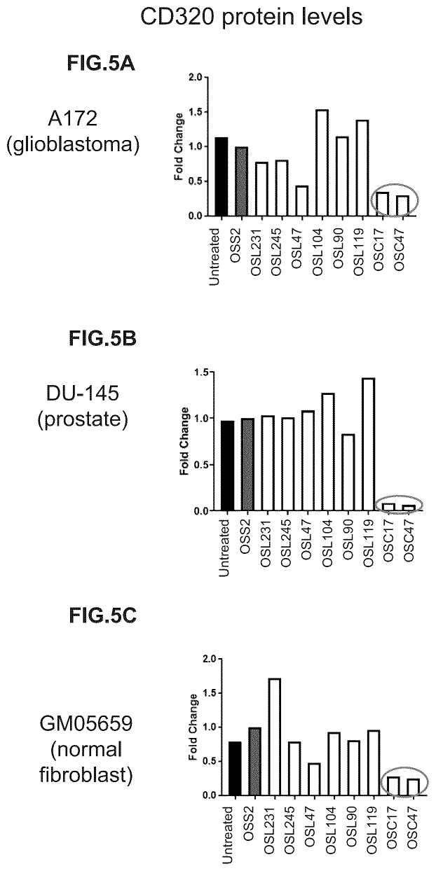

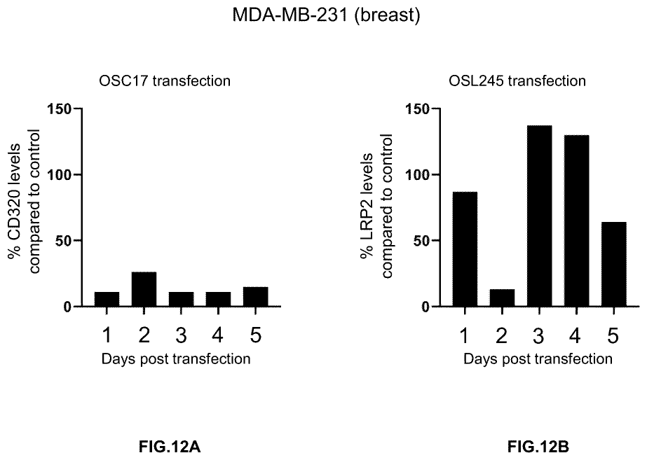

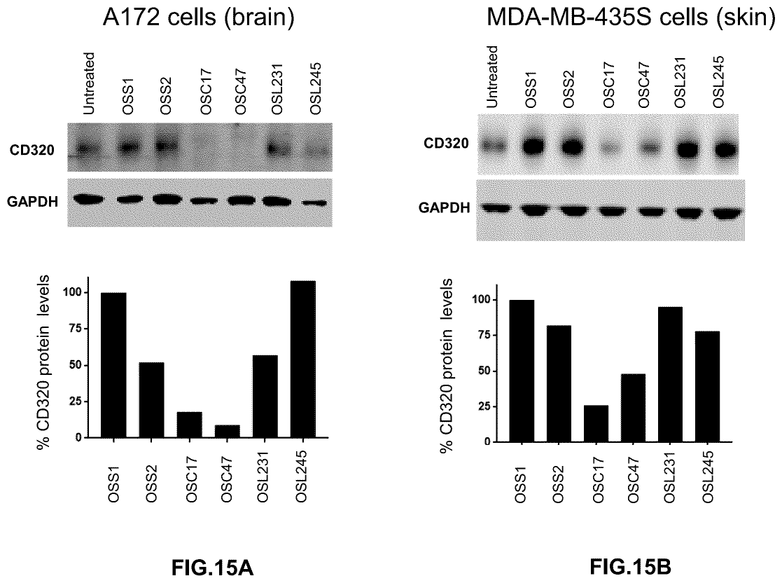

siRNA sequences that efficiently knock down the protein levels of LRP2 and/or CD320 were designed and identified. Table 4 is a list of siRNA sequences complementary to mRNA for CD320 or LRP2 that were tested for their ability to knock down CD320 or LRP2 protein, respectively (see FIG. 3, FIG. 4, FIG. 5, FIG. 12, and FIG. 15).

TABLE 4

| | | | | Nucleotide | |
|---|---|---|---|---|---|
| ID | Passenger Sequence | Target | Size | start site | Location |
| OSS1 | CCUAAGGUUAAGUCGCCCUCG | none | 21 | N/A | N/A |
| | (SEQ ID NO. 947) | | | | |
| OSS2 | UGGUUUACAUGUUGUGUGA | none | 19 | N/A | N/A |
| | (SEQ ID NO. 948) | | | | |
| OSL231 | GGGCUCUAGGUUUGGUGCUAU | LRP2 | 25 | 12537 | CDS |
| | CAAA (SEQ ID NO. 949) | NM_004525.2 | | | |
| OSL245 | GGACUGAUAGGAGAGU | LRP2 | 25 | 12995 | CDS |
| | CAUUGCAAA (SEQ ID NO. 950) | NM_004525.2 | | | |
| OSL47 | CCUGUAAUAAACACUACUCUU | LRP2 | 21 | 2800 | CDS |
| | (SEQ ID NO. 951) | NM_004525.2 | | | |
| OSL104 | CCUUCUAUGAACCUGGCCUUA | LRP2 | 21 | 5677 | CDS |
| | (SEQ ID NO. 952) | NM_004525.2 | | | |
| OSL90 | GUGAUUUGAUUAUACGGCA | LRP2 | 19 | 5126 | CDS |
| | (SEQ ID NO. 953) | NM_004525.2 | | | |
| OSL119 | CCUCAAAUGGCUGUAGCAA | LRP2 | 19 | 6266 | CDS |
| | (SEQ ID NO. 954) | NM_004525.2 | | | |
| OSC17 | GAACUGACAAGAAACUGCGCAACUG | CD320 | 25 | 422 | CDS |
| | (SEQ ID NO. 955) | NM_016579.3 | | | |
| OSC47 | CCCUCAGAGACCUGAGCUCUU | CD320 | 21 | 1006 | 3'-UTR |
| | (SEQ ID NO. 956) | NM_016579.3 | | | |

The list of all potential siRNA sequences is quite large. We have identified 340 potential SIRNA sequences to LRP2 and 59 potential siRNA sequences to CD320. (See Table 5 and Table 6 for the complete list and Table 5A and Table 6A identify the target position and sequence that is complementary for each antisense sequence identified). In addition, chemical modifications can be made to these siRNA sequences to improve their stability and reduce their off-target effects. siRNA molecules are vulnerable to metabolic degradation, for example by RNase or DNase enzymes. Chemical modification of siRNA molecules by incorporation of one or more unnatural, that is, manmade, nucleotides within the sequence can render siRNAs resistant to such metabolic degradation and increase their biological half-life in the cell or in plasma. Moreover, the inclusion of manmade nucleotides at strategic locations within the siRNA sequence can decrease the immunogenicity of the siRNA and improve the selectivity for the guide strand over the passenger strand. Modified siRNA molecules may incorporate manmade nucleotides of a single type or may include multiple manmade nucleotides of different types. Manmade nucleotides may include, but are not limited to, those which contain chemical modifications to the ribose moiety or to the phosphate moieties (FIG. 19 and Table 7). Examples of manmade nucleotides include, but are not limited to, the structures shown in the Table 7. Moreover, modification of multiple structural elements may be combined. In addition, modification may be made to the nucleobase B, which, in addition to the natural RNA nucleobases (G, C, A, U), may include unnatural bases, such as those containing a sulfur atom (e.g., thiouracil).

TABLE 7
Nucleotide modifications corresponding to FIG. 19A

| | | | | | | B |
|---|---|---|---|---|---|---|
| Designation^{a} | Y | X | Z | R | R′ | (nucleobase) |
| [2fN] | O | O | O | F | H | G, C, A, U, |
| | | | | | | other |
| 2′-FANA | O | O | O | H | F | G, C, A, U, |
| | | | | | | other |
| [mN] | O | O | O | OMe | H | G, C, A, U, |
| | | | | | | other |
| 2′-MOE | O | O | O | CH₂CH₂OMe | H | G, C, A, U, |
| | | | | | | other |
| 2′-EA | O | O | O | CH₂CH₂NH₂ | H | G, C, A, U, |
| | | | | | | other |
| 2′-DMEA | O | O | O | CH₂CH₂NMe₂ | H | G, C, A, U, |
| | | | | | | other |
| 2′-DMAP | O | O | O | CH₂CH₂CH₂NMe₂ | H | G, C, A, U, |
| | | | | | | other |
| * as in N1*N2 | O | S | O | OH | H | G, C, A, U, |
| | | | | | | other |
| ** as in N1**N2 | S | S | O | OH | H | G, C, A, U, |
| | | | | | | other |
| 2′-deoxy | O | O | O | H | H | G, C, A, U, |
| | | | | | | other |
| 4′-S | O | O | S | H | H | G, C, A, U, |
| | | | | | | other |
| F-SRNA | O | O | S | F | H | G, C, A, U, |
| | | | | | | other |
| Me-SRNA | O | O | S | OMe | H | G, C, A, U, |
| | | | | | | other |
| 4′-S-FANA | O | O | S | H | F | G, C, A, U, |
| | | | | | | other |

^{a}N designates an arbitrary ribonucleotide or deoxyribonucleotide or analogs thereof.

In some embodiments, chemical modification is made to the phosphodiester group which covalently connects two nucleotides, such that, for example, one or two oxygen atoms in that group are substituted with sulfur atoms, as indicated by a single or double asterisk between two nucleotides to represent the replacement of one or two oxygen atoms with sulfur in the phosphodiester (Table 7 and FIG. 19A). In some embodiments, the siRNA sequences may include other manmade nucleotides wherein further structural modifications have been made to the ribose moiety, such as the addition of bridging atoms that covalently link carbons 2′ and 5′ of the ribose moiety (FIG. 19B-C) or positions 1′ and 2′ of the ribose moiety (FIG. 19D), or alternatively, changes to the size of the sugar ring in a given nucleotide, for example, deletion of the bond between carbons 2′ and 3′ of the ribose moiety (FIG. 19E), or increasing the size of the sugar ring from five to six atoms (FIG. 19F-G).

TABLE 5
CD320

| OS ID | Antisense Strand (5′ TO 3′) | OSID | Sense Strand (5[′ TO 3′) |
|---|---|---|---|
| OSC1A | UCUUAUCCCUGCGCACGCGCA[dT][dT] (SEQ | OSC1S | UGCGCGUGCGCAGGGAUAAGA[dT][dT] |
| | ID NO 1) | | (SEQ ID NO: 94) |
| OSC2A | UCUCUUAUCCCUGCGCACGCG[dT][dT] (SEQ | OSC2S | CGCGUGCGCAGGGAUAAGAGA[dT][dT] |
| | ID NO 2) | | (SEQ ID NO: 95) |
| OSC3A | AUGCUGUCCCCACAGCGGCGC[dT][dT] (SEQ | OSC3S | GCGCCGCUGUGGGGACAGCAU[dT][dT] |
| | ID NO 3) | | (SEQ ID NO: 96) |
| OSC4A | AUCCAACCGCCGCUCAUGCUG[dT][dT] (SEQ | OSC4S | CAGCAUGAGCGGCGGUUGGAU[dT][dT] |
| | ID NO 4) | | (SEQ ID NO: 97) |
| OSC5A | UGGAAAGCGGGCUCGCGGCGG[dT][dT] (SEQ | OSC5S | CCGCCGCGAGCCCGCUUUCCA[dT][dT] |
| | ID NO 5) | | (SEQ ID NO: 98) |
| OSC6A | AACUUGGUGGGUGGGCACGAG[dT][dT] (SEQ | OSC6S | CUCGUGCCCACCCACCAAGUU[dT][dT] |
| | ID NO 6) | | (SEQ ID NO: 99) |
| OSC7A | UGGAACUUGGUGGGUGGGCAC[dT][dT] (SEQ | OSC7S | GUGCCCACCCACCAAGUUCCA[dT][dT] |
| | ID NO 7) | | (SEQ ID NO: 100) |
| OSC8A | ACUGGAACUUGGUGGGUGGGC[dT][dT] (SEQ | OSC8S | GCCCACCCACCAAGUUCCAGU[dT][dT] |
| | ID NO 8) | | (SEQ ID NO: 101) |
| OSC9A | UAAGCCACUGGUGCGGCACUG[dT][dT] (SEQ | OSC9S | CAGUGCCGCACCAGUGGCUUA[dT][dT] |
| | ID NO 9) | | (SEQ ID NO: 102) |
| OSC10A | ACGCAUAAGCCACUGGUGCGG[dT][dT] (SEQ | OSC10S | CCGCACCAGUGGCUUAUGCGU[dT][dT] |
| | ID NO 10) | | (SEQ ID NO: 103) |
| OSC11A | UCCAAGUCCCUGUCGCAGCGC[dT][dT] (SEQ | OSC11S | GCGCUGCGACAGGGACUUGGA[dT][dT] |
| | ID NO 11) | | (SEQ ID NO: 104) |
| OSC12A | UCCUCAUCGCUGCCAUCGCUG[dT][dT] (SEQ | OSC12S | CAGCGAUGGCAGCGAUGAGGA[dT][dT] |
| | ID NO 12) | | (SEQ ID NO: 105) |
| OSC13A | UCACUGACGCCGGUGCAGGGG[dT][dT] (SEQ | OSC13S | CCCCUGCACCGGCGUCAGUGA[dT][dT] |
| | ID NO 13) | | (SEQ ID NO: 106) |
| OSC14A | UUGUCAGUUCCCCCAGAGCAG[dT][dT] (SEQ | OSC14S | CUGCUCUGGGGGAACUGACAA[dT][dT] |
| | ID NO 14) | | (SEQ ID NO: 107) |
| OSC15A | UUCUUGUCAGUUCCCCCAGAG[dT][dT] (SEQ | OSC15S | CUCUGGGGGAACUGACAAGAA[dT][dT] |
| | ID NO 15) | | (SEQ ID NO: 108) |
| OSC16A | AGUUUCUUGUCAGUUCCCCCA[dT][dT] (SEQ | OSC16S | UGGGGGAACUGACAAGAAACU[dT][dT] |
| | ID NO 16) | | (SEQ ID NO: 109) |
| OSC17A- | CAGUUGCGCAGUUUCUUGUCAGUUC[dT][dT] | OSC17S- | GAACUGACAAGAAACUGCGCAACUG |
| 1 | (SEQ ID NO 17) | 1 | [dT][dT] (SEQ ID NO: 110) |
| OSC17A- | CAGUUGCGCAGUUUCUUGUCAGUUC[dT]*[dT] | OSC17S- | GAACUGACAAGAAACUGCGCAACUG[dT]* |
| 2 | (SEQ ID NO 18) | 2 | [dT] (SEQ ID NO: 111) |
| OSC17A- | [mC][mA][mG][mU][mU][mG][mC][mG][mC] | OSC17S- | [mG][mA][mA][mC][mU][mG][mA][mC] |
| 3 | [mA][mG][mU][mU][mU][mC][mU][mU][mG] | 3 | [mA][mA][mG][mA][mA][mA][mC][mU] |
| | [mU][mC][mA][mG][mU][mU][mC][dT]*[dT] | | [mG][mC][mG][mC][mA][mA][mC][mU][mG] |
| | (SEQ ID NO 19) | | [dT]*[dT] (SEQ ID NO: 112) |
| OSC17A- | [mC][mA][mG][mU][mU][mG][mC][mG][mC] | OSC17S- | [mG][mA][mA][mC][mU][mG][mA][mC] |
| 4 | [mA][mG][mU][mU][mU][mC][mU][mU][mG] | 4 | [mA][mA][mG][mA][mA][mA][mC][mU] |
| | [mU][mC][mA][mG][mU][mU][mC][dT]*[dT] | | [mG][mC][mG][mC][mA][mA][mC][mU][mG] |
| | (SEQ ID NO 20) | | (SEQ ID NO: 113) |
| OSC17A- | [mC][mA][mG][mU][mU][mG][mC][mG][mC] | OSC17S- | [mG][mA][mA][mC][mU][mG][mA][mC] |
| 5 | [mA][mG][mU][mU][mU][mC][mU][mU][mG] | 5 | [mA][mA][mG][mA][mA][mA][mC][mU] |
| | [mU][mC][mA][mG][mU][mU][mC] (SEQ ID | | [mG][mC][mG][mC][mA][mA][mC][mU][mG] |
| | NO 21) | | [dT]*[dT] (SEQ ID NO: 114) |
| OSC17A- | [mC][mA][mG][mU][mU][mG][mC][mG][mC] | OSC17S- | [mG][mA][mA][mC][mU][mG][mA][mC] |
| 6 | [mA][mG][mU][mU][mU][mC][mU][mU][mG] | 6 | [mA][mA][mG][mA][mA][mA][mC][mU] |
| | [mU][mC][mA][mG][mU][mU][mC] (SEQ ID | | [mG][mC][mG][mC][mA][mA][mC][mU][mG] |
| | NO 22) | | (SEQ ID NO: 115) |
| OSC17A- | [mC][mA][mG][mU][mU][mG][mC][mG][mC] | OSC17S- | GAACUGACAAGAAACUGCGCAACUG[dT]* |
| 7 | [mA][mG][mU][mU][mU][mC][mU][mU][mG] | 7 | [dT] (SEQ ID NO: 116) |
| | [mU][mC][mA][mG][mU][mU][mC] | | |
| | [dT]*[dT] (SEQ ID NO 23) | | |
| OSC17A- | [mC][2fA][mG][2fU][mU][2fG][mC] | OSC17S- | [2fG][mA][2fA][mC][2fU][mG][2fA][mC] |
| 8 | [2fG][mC][2fA][mG][2fU][mU][2fU][mC] | 8 | [2fA][mA][2fG][mA][2fA][mA][2fC][mU] |
| | [2fU][mU][2fG][mU][2fC][mA][2fG][mU] | | [2fG][mC][2fG][mC][2fA][mA][2fC][mU] |
| | [2fU][mC][dT]*[dT] (SEQ ID NO 24) | | [2fG][dT]*[[dT] (SEQ ID NO: 117) |
| OSC17A- | [mC][2fA][mG][2fU][mU][2fG][mC][2fG] | OSC17S- | [2fG][mA][2fA][mC][2fU][mG][2fA][mC] |
| 9 | [mC][2fA][mG][2fU][mU][2fU][mC] | 9 | [2fA][mA][2fG][mA][2fA][mA][2fC][mU] |
| | [2fU][mU][2fG][mU][2fC][mA][2fG][mU] | | [2fG][mC][2fG][mC][2fA][mA][2fC][mU] |
| | [2fU][mC] (SEQ ID NO 25) | | [2fG][dT]*[dT] (SEQ ID NO: 118) |
| OSC17A- | [mC][2fA][mG][2fU][mU][2fG][mC][2fG] | OSC17S- | [2fG][mA][2fA][mC][2fU][mG][2fA][mC] |
| 10 | [mC][2fA][mG][2fU][mU][2fU][mC][2fU] | 10 | [2fA][mA][2fG][mA][2fA][mA][2fC][mU] |
| | [mU][2fG][mU][2fC][mA][2fG][mU][2fU] | | [2fG][mC][2fG][mC][2fA][mA][2fC][mU] |
| | [mC][dT]*[dT] (SEQ ID NO 26) | | [2fG] (SEQ ID NO: 119) |
| OSC17A- | [mC][2fA][mG][2fU][mU][2fG][mC][2fG] | OSC17S- | [2fG][mA][2fA][mC][2fU][mG][2fA][mC] |
| 11 | [mC][2fA][mG][2fU][mU][2fU][mC] | 11 | [2fA][mA][2fG][mA][2fA][mA][2fC][mU] |
| | [2fU][mU][2fG][mU][2fC][mA][2fG][mU] | | [2fG][mC][2fG][mC][2fA][mA][2fC][mU] |
| | [2fU][mC] (SEQ ID NO 27) | | [2fG] (SEQ ID NO: 120) |
| OSC17A- | [2fC][mA][2fG][mU][2fU][mG][2fC][mG] | OSC17S- | [mG][2fA][mA][2fC][mU][2fG][mA][2fC] |
| 12 | [2fC][mA][2fG][mU][2fU][mU][2fC][mU] | 12 | [mA][2fA][mG][2fA][mA][2fA][mC][2fU] |
| | [2fU][mG][2fU][mC][2fA][mG][2fU][mU] | | [mG][2fC][mG][2fC][mA][2fA][mC][2fU] |
| | [2fC][dT]*[dT](SEQ ID NO 28) | | [mG][dT]*[dT] (SEQ ID NO: 121) |
| OSC17A- | [2fC][mA][2fG][mU][2fU][mG][2fC][mG] | OSC17S- | [mG][2fA][mA][2fC][mU][2fG][mA][2fC] |
| 13 | [2fC][mA][2fG][mU][2fU][mU][2fC][mU] | 13 | [mA][2fA][mG][2fA][mA][2fA][mC] |
| | [2fU][mG][2fU][mC][2fA][mG][2fU] | | [2fU][mG][2fC][mG][2fC][mA][2fA][mC] |
| | [mU][2fC](SEQ ID NO 29) | | [2fU][mG] (SEQ ID NO: 122) |
| OSC17A- | [mC][2fA][2fG][2fU][2fU][2fG][2fC][2fG] | OSC17S- | [2fU][mG][2fA][mC][2fA][mA][2fG][mA] |
| 14 | [2fC][2fA][2fG][2fU][2fU][2fU][2fC] | 14 | [2fA][mA][2fC][mU][2fG][mC][2fG][mC] |
| | [2fU][2fU][2fG][2fU][2fC][2fA][2fG] | | [2fA][mA][2fC][mU][2fG][dT]*[dT] |
| | [2fU][2fU][2fC][dT]*[dT] (SEQ ID NO 30) | | (SEQ ID NO: 123) |
| OSC17A- | [mC][2fA][2fG][2fU][2fU][2fG][2fC] | OSC17S- | [2fG][mA][2fA][mA][2fC][mU][2fG][mC] |
| 15 | [2fG][2fC][2fA][2fG][2fU][2fU][2fU] | 15 | [2fG][mC][2fA][mA][2fC][mU][2fG] |
| | [2fC][2fU][2fU][2fG][2fU][2fC][2fA] | | [dT]*[dT] (SEQ ID NO: 124) |
| | [2fG][2fU][2fU][2fC][dT]*[dT] | | |
| | (SEQ ID NO 31) | | |
| OSC17A- | [mC][2fA][mG][2fU][mU][2fG][mC][2fG] | OSC17S- | [2fG][mA][2fA][mA][2fC][mU][2fG][mC] |
| 16 | [mC][2fA][mG][2fU][mU][2fU][mC][2fU] | 16 | [2fG][mC][2fA][mA][2fC][mU][2fG] |
| | [mU][2fG][mU][2fC][mA][2fG][mU][2fU] | | [dT]*[dT] (SEQ ID NO: 125) |
| | [mC][dT]*[dT] (SEQ ID NO 32) | | |
| OSC17A- | [mC][2fA][mG][2fU][mU][2fG][mC] | OSC17S- | [2fG][mA][2fA][mA][2fC][mU][2fG][mC] |
| 17 | [2fG][mC][2fA][mG][2fU][mU][2fU][mC] | 17 | [2fG][mC][2fA][mA][2fC][mU][2fG] |
| | [2fU][mU][2fG][mU][2fC][mA][2fG] | | (SEQ ID NO: 126) |
| | [mU] (SEQ ID NO 33) | | |
| OSC17A- | [mC][2fA][mG][2fU][mU][2fG][mC][2fG] | OSC17S- | [2fG][mA][2fA][mA][2fC][mU][2fG][mC] |
| 18 | [mC][2fA][mG][2fU][mU][2fU][mC] | 18 | [2fG][mC][2fA][mA][2fC][mU][2fG] |
| | [2fU][2fU][2fG][2fU][2fC][2fA][2fG] | | (SEQ ID NO: 127) |
| | [2fU] (SEQ ID NO 34) | | |
| OSC18A | AUGCAGUCAUCGCUCAGCGUG[dT][dT] (SEQ | OSC18S | CACGCUGAGCGAUGACUGCAU[dT][dT] |
| | ID NO 35) | | (SEQ ID NO: 128) |
| OSC19A | AAUGCAGUCAUCGCUCAGCGU[dT][dT] (SEQ | OSC19S | ACGCUGAGCGAUGACUGCAUU[dT][dT] |
| | ID NO 36) | | (SEQ ID NO: 129) |
| OSC20A | UGGAAUGCAGUCAUCGCUCAG[dT][dT] (SEQ | OSC20S | CUGAGCGAUGACUGCAUUCCA[dT][dT] |
| | ID NO 37) | | (SEQ ID NO: 130) |
| OSC21A | AGUGGAAUGCAGUCAUCGCUC[dT][dT] (SEQ | OSC21S | GAGCGAUGACUGCAUUCCACU[dT][dT] |
| | ID NO 38) | | (SEQ ID NO: 131) |
| OSC22A | ACAGUCUGGGUGGCCGUCGCA[dT][dT] (SEQ | OSC22S | UGCGACGGCCACCCAGACUGU[dT][dT] |
| | ID NO 39) | | (SEQ ID NO: 132) |
| OSC23A | AUUGGUUCCACAGCCGAGCUC[dT][dT] (SEQ | OSC23S | GAGCUCGGCUGUGGAACCAAU[dT][dT] |
| | ID NO 40) | | (SEQ ID NO: 133) |
| OSC24A | UCUCAUUGGUUCCACAGCCGA[dT][dT] (SEQ | OSC24S | UCGGCUGUGGAACCAAUGAGA[dT][dT] |
| | ID NO 41) | | (SEQ ID NO: 134) |
| OSC25A | AUCUCAUUGGUUCCACAGCCG[dT][dT] (SEQ | OSC25S | CGGCUGUGGAACCAAUGAGAU[dT][dT] |
| | ID NO 42) | | (SEQ ID NO: 135) |
| OSC26A | AGGAUCUCAUUGGUUCCACAG[dT][dT] (SEQ | OSC26S | CUGUGGAACCAAUGAGAUCCU[dT][dT] |
| | ID NO 43) | | (SEQ ID NO: 136) |
| OSC27A | UGAGAGAGGUGACACUCUCCA[dT][dT] (SEQ | OSC27S | UGGAGAGUGUCACCUCUCUCA[dT][dT] |
| | ID NO 44) | | (SEQ ID NO: 137) |
| OSC28A | UGGUUGUGGCAUUCCUGAGAG[dT][dT] | OSC28S | CUCUCAGGAAUGCCACAACCA[dT][dT] |
| | (SEQ ID NO 45) | | (SEQ ID NO: 138) |
| OSC29A | UGGCAUUCCCGACAGAGGGGA[dT][dT] (SEQ | OSC29S | UCCCCUCUGUCGGGAAUGCCA[dT][dT] |
| | ID NO 46) | | (SEQ ID NO: 139) |
| OSC30A | AGGAUGUGGCAUUCCCGACAG[dT][dT] (SEQ | OSC30S | CUGUCGGGAAUGCCACAUCCU[dT][dT] |
| | ID NO 47) | | (SEQ ID NO: 140) |
| OSC31A | UUCCAGACUGGUCUCCGGCAG[dT][dT] (SEQ | OSC31S | CUGCCGGAGACCAGUCUGGAA[dT][dT] |
| | ID NO 48) | | (SEQ ID NO: 141) |
| OSC32A | AUAACCCCAUAGGCAGUUGGG[dT][dT] (SEQ | OSC32S | CCCAACUGCCUAUGGGGUUAU[dT][dT] |
| | ID NO 49) | | (SEQ ID NO: 142) |
| OSC33A | UUGCACUGAGCACCGCAGCAG[dT][dT] (SEQ | OSC33S | CUGCUGCGGUGCUCAGUGCAA[dT][dT] |
| | ID NO 50) | | (SEQ ID NO: 143) |
| OSC34A | AAAAGGAGGAGGGUGGCGGUG[dT][dT] (SEQ | OSC34S | CACCGCCACCCUCCUCCUUUU[dT][dT] |
| | ID NO 51) | | (SEQ ID NO: 144) |
| OSC35A | ACAAAAGGAGGAGGGUGGCGG[dT][dT] (SEQ | OSC35S | CCGCCACCCUCCUCCUUUUGU[dT][dT] |
| | ID NO 52) | | (SEQ ID NO: 145) |
| OSC36A | ACCAGUAACCCCAGUGGGCGG[dT][dT] (SEQ | OSC36S | CCGCCCACUGGGGUUACUGGU[dT][dT] |
| | ID NO 53) | | (SEQ ID NO: 146) |
| OSC37A | UUCAUGGCCACCAGUAACCCC[dT][dT] (SEQ | OSC37S | GGGGUUACUGGUGGCCAUGAA[dT][dT] |
| | ID NO 54) | | (SEQ ID NO: 147) |
| OSC38A | ACUCCUUCAUGGCCACCAGUA[dT][dT] (SEQ | OSC38S | UACUGGUGGCCAUGAAGGAGU[dT][dT] |
| | ID NO 55) | | (SEQ ID NO: 148) |
| OSC39A | UUCUGACAGCAGCAGGGACUC[dT][dT] (SEQ | OSC39S | GAGUCCCUGCUGCUGUCAGAA[dT][dT] |
| | ID NO 56) | | (SEQ ID NO: 149) |
| OSC40A | UCUGUUCUGACAGCAGCAGGG[dT][dT] (SEQ | OSC40S | GGCUGCUGCUGUCAGAACAGA[dT][dT] |
| | ID NO 57) | | (SEQ ID NO: 150) |
| OSC41A | UCUUCUGUUCUGACAGCAGCA[dT][dT] (SEQ | OSC41S | UGCUGCUGUCAGAACAGAAGA[dT][dT] |
| | ID NO 58) | | (SEQ ID NO: 151) |
| OSC42A | AGGUCUUCUGUUCUGACAGCA[dT][dT] (SEQ | OSC42S | UGCUGUCAGAACAGAAGACCU[dT][dT] |
| | ID NO 59) | | (SEQ ID NO: 152) |
| OSC43A | UUGUCCUCAGGGCAGCGAGGU[dT][dT] (SEQ | OSC43S | ACCUCGCUGCCCUGAGGACAA[dT][dT] |
| | ID NO 60) | | (SEQ ID NO: 153) |
| OSC44A | AAGUGCUUGUCCUCAGGGCAG[dT][dT] (SEQ | OSC44S | CUGCCCUGAGGACAAGCACUU[dT][dT] |
| | ID NO 61) | | (SEQ ID NO: 154) |
| OSC45A | UACCCAUCCGCAUCACUGCUC[dT][dT] (SEQ | OSC45S | GAGCAGUGAUGCGGAUGGGUA[dT][dT] |
| | ID NO 62) | | (SEQ ID NO: 155) |
| OSC46A | UCUCUGAGGGCUGGUGUGCCC[dT][dT] (SEQ | OSC46S | GGGCACACCAGCCCUCAGAGA[dT][dT] |
| | ID NO 63) | | (SEQ ID NO: 156) |
| OSC47A- | AAGAGCUCAGGUCUCUGAGGG[dT][dT] (SEQ | OSC47S- | CCCUCAGAGACCUGAGCUCUU[dT][dT] |
| 1 | ID NO 64) | 1 | (SEQ ID NO: 157) |
| OSC47A- | AAGAGCUCAGGUCUCUGAGGG[dT]*[dT] | OSC47S- | CCCUCAGAGACCUGAGCUCUU[dT]*[dT] |
| 2 | (SEQ ID NO 65) | 2 | (SEQ ID NO: 158) |
| OSC47A- | [mA][mA][mG][mA][mG][mC][mU][mC][mA] | OSC47S- | [mC][mC][mC][mU][mC][mA][mG][mA] |
| 3 | [mG][mG][mU][mC][mU][mC][mU][mG][mA] | 3 | [mG][mA][mC][mC][mU][mG][mA][mG] |
| | [mG][mG][mG][dT]*[dT] (SEQ ID NO 66) | | [mC][mU][mC][mU][mU][dT]*[dT] |
| | | | (SEQ ID NO: 159) |
| OSC47A- | [mA][mA][mG][mA][mG][mC][mU][mC][mA] | OSC47S- | [mC][mC][mC][mU][mC][mA][mG][mA] |
| 4 | [mG][mG][mU][mC][mU][mC][mU][mG][mA] | 4 | [mG][mA][mC][mC][mU][mG][mA][mG] |
| | [mG][mG][mG][dT]*[dT] (SEQ ID NO 67) | | [mC][mU][mC][mU][mU] |
| | | | (SEQ ID NO: 160) |
| OSC47A- | [mA][mA][mG][mA][mG][mC][mU][mC][mA] | OSC47S- | [mC][mC][mC][mU][mC][mA][mG][mA] |
| 5 | [mG][mG][mU][mC][mU][mC][mU][mG][mA] | 5 | [mG][mA][mC][mC][mU][mG][mA][mG] |
| | [mG][mG][mG] (SEQ ID NO 68) | | [mC][mU][mC][mU][mU][dT]*[dT] |
| | | | (SEQ ID NO: 161) |
| OSC47A- | [mA][mA][mG][mA][mG][mC][mU][mC][mA] | OSC47S- | [mC][mC][mC][mU][mC][mA][mG][mA] |
| 6 | [mG][mG][mU][mC][mU][mC][mU][mG][mA] | 6 | [mG][mA][mC][mC][mU][mG][mA][mG] |
| | [mG][mG][mG] (SEQ ID NO 69) | | [mC][mU][mC][mU][mU] |
| | | | (SEQ ID NO: 162) |
| OSC47A- | [mA][mA][mG][mA][mG][mC][mU][mC][mA] | OSC47S- | CCCUCAGAGACCUGAGCUCUU[dT]*[dT] |
| 7 | [mG][mG][mU][mC][mU][mC][mU][mG][mA] | 7 | (SEQ ID NO: 163) |
| | [mG][mG][mG][dT]*[dT] (SEQ ID NO 70) | | |
| OSC47A- | [mA][2fA][mG][2fA][mG][2fC][mU][2fC] | OSC47S- | [2fC][mC][2fC][mU][2fC][mA][2fG][mA] |
| 8 | [mA][2fG][mG][2fU][mC][2fU][mC] | 8 | [2fG][mA][2fC][mC][2fU][mG][2fA][mG] |
| | [2fU][mG][2fA][mG][2fG][mG][dT]* | | [2fC][mU][2fC][mU][2fU][dT]*[dT] |
| | [dT] (SEQ ID NO 71) | | (SEQ ID NO: 164) |
| OSC47A- | [mA][2fA][mG][2fA][mG][2fC][mU][2fC] | OSC47S- | [2fC][mC][2fC][mU][2fC][mA][2fG][mA] |
| 9 | [mA][2fG][mG][2fU][mC][2fU] | 9 | [2fG][mA][2fC][mC][2fU][mG][2fA][mG] |
| | [mC][2fU][mG][2fA][mG][2fG] | | [2fC][mU][2fC][mU][2fU][dT]*[dT] |
| | [mG] (SEQ ID NO 72) | | (SEQ ID NO: 165) |
| OSC47A- | [mA][2fA][mG][2fA][mG][2fC][mU][2fC] | OSC47S- | [2fC][mC][2fC][mU][2fC][mA][2fG][mA] |
| 10 | [mA][2fG][mG][2fU][mC][2fU][mC][2fU] | 10 | [2fG][mA][2fC][mC][2fU][mG][2fA][mG] |
| | [mG][2fA][mG][2fG][mG][dT]*[dT] | | [2fC][mU][2fC][mU][2fU] |
| | (SEQ ID NO 73) | | (SEQ ID NO: 166) |
| OSC47A- | [mA][2fA][mG][2fA][mG][2fC][mU][2fC] | OSC47S- | [2fC][mC][2fC][mU][2fC][mA][2fG] |
| 11 | [mA][2fG][mG][2fU][mC][2fU][mC][2fU] | 11 | [mA][2fG[mA][2fC][mC][2fU][mG][2fA] |
| | [mG][2fA][mG][2fG][mG] | | [mG][2fC][mU][2fC][mU][2fU] |
| | (SEQ ID NO 74) | | (SEQ ID NO: 167) |
| OSC47A- | [2fA][mA][2fG][mA][2fG][mC][2fU][mC] | OSC47S- | [mC][2fC][mC][2fU][mC][2fA][mG][2fA] |
| 12 | [2fA][mG][2fG][mU][2fC][mU][2fC] | 12 | [mG][2fA][mC][2fC][mU][2fG][mA][2fG] |
| | [mU][2fG][mA][2fG][mG][2fG][dT]*[dT] | | [mC][2fU][mC][2fU][mU][dT][dT]* |
| | (SEQ ID NO 75) | | (SEQ ID NO: 168) |
| OSC47A- | [2fA][mA][2fG][mA][2fG][mC][2fU][mC] | OSC47S- | [2fC][mC][2fC][mU][2fC][mA][2fG][mA] |
| 13 | [2fA][mG][2fG][mU][2fC][mU][2fC] | 13 | [2fG][mA][2fC][mC][2fU][mG][2fA][mG] |
| | [mU][2fG][mA][2fG][mG][2fG] | | [2fC][mU][2fC][mU][2fU]-LIG-LINKER |
| | (SEQ ID NO 76) | | (SEQ ID NO: 169) |
| OSC47A- | [mA][2fA][2fG][mA][2fG][mC][2fU][mC] | OSC47S- | [2fC][mC][2fC][mU][2fC][mA][2fG][mA] |
| 14 | [2fA][mG][2fG][mU][2fC][mU][2fC][mU] | 14 | [2fG][mA][2fC][mC][2fU][mG][2fA] |
| | [2fG][mA][2fG][mG][2fG] | | [mG][2fC][mU][2fC][mU][2fU][dT]*[dT] |
| | (SEQ ID NO 77) | | (SEQ ID NO: 170) |
| OSC47A- | [mA][2fA][2fG][mA][2fG][mC][2fU][mC] | OSC47S- | [2fG][mA][2fG][mA][2fC][mC][2fU][mG] |
| | [2fA][mG][2fG][mU][2fC][mU][2fC][mU] | 15 | [2fA][mG][2fC][mU][2fC][mU][2fU] |
| 15 | [2fG][mA][2fG][mG][2fG][dT]*[dT] | | [dT]*[dT] (SEQ ID NO: 171) |
| | (SEQ ID NO 78) | | |
| OSC47A- | [2fA][mA][2fG][mA][2fG][mC][2fU][mC] | OSC47S- | [2fG][mA][2fG][mA][2fC][mC][2fU][mG] |
| 16 | [2fA][mG][2fG][mU][2fC][mU][2fC][mU] | 16 | [2fA][mG][2fC][mU][2fC][mU][2fU] |
| | [2fG][mA][2fG][mG][2fG][dT]*[dT] | | [dT]*[dT] (SEQ ID NO: 172) |
| | (SEQ ID NO 79) | | |
| OSC47A- | [2fA][mA][2fG][mA][2fG][mC][2fU][mC] | OSC47S- | [2fG][mA][2fG][mA][2fC][mC][2fU][mG] |
| 17 | [2fA][mG][2fG][mU][2fC][mU][2fC][mU] | 17 | [2fA][mG][2fC][mU][2fC][mU][2fU] |
| | [2fG][mA][2fG][mG][2fG][dT]*[dT] | | (SEQ ID NO: 173) |
| | (SEQ ID NO 80) | | |
| OSC47A- | [2fA][mA][2fG][mA][2fG][mC][2fU] | OSC47S- | [2fG][mA][2fG][mA][2fC][mC][2fU][mG] |
| 18 | [mC][2fA][mG][2fG][mU][2fC][mU] | 18 | [2fA][mG][2fC][mU][2fC][mU][2fU] |
| | [2fC][2fU][2fG][2fA][2fG][2fG][2fG] | | (SEQ ID NO: 174) |
| | (SEQ ID NO 81) | | |
| OSC48A | AAGAGCUCAGGUCUCUGAGGG[dT][dT] (SEQ | OSC48S | CCCUCAGAGACCUGAGCUCUU[dT][dT] |
| | ID NO 82) | | (SEQ ID NO: 175) |
| OSC49A | AGAAGAGCUCAGGUCUCUGAG[dT][dT] (SEQ | OSC49S | CUCAGAGACCUGAGCUCUUCU[dT][dT] |
| | ID NO: 83) | | (SEQ ID NO: 176) |
| OSC50A | AUAGGGAGUGUCCAGGGACCC[dT][dT] (SEQ | OSC50S | GGGUCCCUGGACACUCCCUAU[dT][dT] |
| | ID NO: 84) | | (SEQ ID NO: 177) |
| OSC51A | UCCAUAGGGAGUGUCCAGGGA[dT][dT] (SEQ | OSC51S | UCCCUGGACACUCCCUAUGGA[dT][dT] |
| | ID NO: 85) | | (SEQ ID NO: 178) |
| OSC52A | AUCUCCAUAGGGAGUGUCCAG[dT][dT] (SEQ | OSC52S | CUGGACACUCCCUAUGGAGAU[dT][dT] |
| | ID NO: 86) | | (SEQ ID NO: 179) |
| OSC53A | UCAGUUCUGGCUGUGGCAGGU[dT][dT] (SEQ | OSC53S | ACCUGCCACAGCCAGAACUGA[dT][dT] |
| | ID NO: 87) | | (SEQ ID NO: 180) |
| OSC54A | UUCUACCCCCUGGGAGCUGCC[dT][dT] (SEQ | OSC54S | GGCAGCUCCCAGGGGGUAGAA[dT][dT] |
| | ID NO: 88) | | (SEQ ID NO: 181) |
| OSC55A | AAGCACAGGGCCGUUCUACCC[dT][dT] (SEQ | OSC55S | GGGUAGAACGGCCCUGUGCUU[dT][dT] |
| | ID NO: 89) | | (SEQ ID NO: 182) |
| OSC56A | UGUCUUAAGCACAGGGCCGUU[dT][dT] (SEQ | OSC56S | AACGGCCCUGUGCUUAAGACA[dT][dT] |
| | ID NO: 90) | | (SEQ ID NO: 183) |
| OSC57A | AGUGUCUUAAGCACAGGGCCG[dT][dT] (SEQ | OSC57S | CGGCCCUGUGCUUAAGACACU[dT][dT] |
| | ID NO: 91) | | (SEQ ID NO: 184) |
| OSC58A | UUUUUUGAGGAUGUGAAGCAA[dT][dT] | OSC58S | UUGCUUCACAUCCUCAAAAAA[dT][dT] |
| | (SEQ ID NO: 92) | | (SEQ ID NO: 185) |
| OSC59A | UUUUUUUGAGGAUGUGAAGCA[dT][dT] | OSC59S | UGCUUCACAUCCUCAAAAAAA[dT][dT] |
| | (SEQ ID NO: 93) | | (SEQ ID NO: 186) |

TABLE 6
LRP2

| OS ID | Antisense Strand (5′ TO 3′) | OS ID | Sense Strand (5′ TO 3′) |
|---|---|---|---|
| OSL1A | UACUUUGUGAGCAAUCUUGAC[dT][dT] (SEQ | OSL1S | GUCAAGAUUGCUCACAAAGUA[dT][dT] |
| | ID NO: 187) | | (SEQ ID NO: 561) |
| OSL2A | AUUCACUUGGGAUACACUGAC[dT][dT] (SEQ | OSL2S | GUCAGUGUAUCCCAAGUGAAU[dT][dT] |
| | ID NO: 188) | | (SEQ ID NO: 562) |
| OSL3A | ACAUGAAAACUCAUUGUGCAA[dT][dT] (SEQ | OSL3S | UUGCACAAUGAGUUUUCAUGU[dT][dT] |
| | ID NO: 189) | | (SEQ ID NO: 563) |
| OSL4A | UCUUUACAGUCAUCUUCUCCA[dT][dT] (SEQ | OSL4S | UGGAGAAGAUGACUGUAAAGAUA[dT][dT |
| | ID NO: 190) | | (SEQ ID NO: 564) |
| OSL5A | UAUCUUUACAGUCAUCUUCUC[dT][dT] (SEQ | OSL5S | GAGAAGAUGACUGUAAAGAUA[dT][dT] |
| | ID NO: 191) | | (SEQ ID NO: 565) |
| OSL6A | AACAUUUAUGAACAUCAUGAG[dT][dT] (SEQ | OSL6S | CUCAUGAUGUUCAUAAAUGUU[dT][dT] |
| | ID NO: 192) | | (SEQ ID NO: 566) |
| OSL7A | UCACAAACUUUAUAAAUGGAG[dT][dT] (SEQ | OSL7S | CUCCAUUUAUAAAGUUUGUGA[dT][dT] |
| | ID NO: 193) | | (SEQ ID NO: 567) |
| OSL8A | AUCACAAACUUUAUAAAUGGA[dT][dT] (SEQ | OSL8S | UCCAUUUAUAAAGUUUGUGAU[dT][dT] |
| | ID NO: 194) | | (SEQ ID NO: 568) |
| OSL9A | AUACUACAGUAUUUUCCGGUA[dT][dT] (SEQ | OSL9S | UACCGGAAAAUACUGUAGUAU[dT][dT] |
| | ID NO: 195) | | (SEQ ID NO: 569) |
| OSL10 | UCAUACUACAGUAUUUUCCGG[dT][dT] (SEQ | OSL10S | CCGGAAAAUACUGUAGUAUGA[dT][dT] |
| A | ID NO: 196) | | (SEQ ID NO: 570) |
| OSL11 | ACAAAUUCCCCAUAUCUGGCA[dT][dT] (SEQ | OSL11S | UGCCAGAUAUGGGGAAUUUGU[dT][dT] |
| A | ID NO: 197) | | (SEQ ID NO: 571) |
| OSL12 | AAGAUAUACCCUUCUUCACAG[dT][dT] (SEQ | OSL12S | CUGUGAAGAAGGGUAUAUCUU[dT][dT] |
| A | ID NO: 198) | | (SEQ ID NO: 572) |
| OSL13 | UUAGCUUUGCAAUACUGUCCA[dT][dT] (SEQ | OSL13S | UGGACAGUAUUGCAAAGCUAA[dT][dT] |
| A | ID NO: 199) | | (SEQ ID NO: 573) |
| OSL14 | AUCAUUAGCUUUGCAAUACUG[dT][dT] (SEQ | OSL14S | CAGUAUUGCAAAGCUAAUGAU[dT][dT] |
| A | ID NO: 200) | | (SEQ ID NO: 574) |
| OSL15 | AAAGGAAUCAUUAGCUUUGCA[dT][dT] (SEQ | OSL15S | UGCAAAGCUAAUGAUUCCUUU[dT][dT] |
| A | ID NO: 201) | | (SEQ ID NO: 575) |
| OSL16 | AUGAAUAUCACCAAUUAACAA[dT][dT] (SEQ | OSL16S | UUGUUAAUUGGUGAUAUUCAU[dT][dT] |
| A | ID NO: 202) | | (SEQ ID NO: 576) |
| OSL17 | AAAAAACCUUAUUUUGCACGG[dT][dT] (SEQ | OSL17S | CCGUGCAAAAUAAGGUUUUUU[dT][dT] |
| A | ID NO: 203) | | (SEQ ID NO: 577) |
| OSL18 | UGAAAAAACCUUAUUUUGCAC[dT][dT] (SEQ | OSL18S | GUGCAAAAUAAGGUUUUUUCA[dT][dT] |
| A | ID NO: 204) | | (SEQ ID NO: 578) |
| OSL19 | AAUGUCAACUGAAAAAACCUU[dT][dT] (SEQ | OSL19S | AAGGUUUUUUCAGUUGACAUU[dT][dT] |
| A | ID NO: 205) | | (SEQ ID NO: 579) |
| OSL20 | UAAUGUCAACUGAAAAAACCU[dT][dT] (SEQ | OSL20S | AGGUUUUUUCAGUUGACAUUA[dT][dT] |
| A | ID NO: 206) | | (SEQ ID NO: 580) |
| OSL21 | UUAAACCAUUAAUGUCAACUG[dT][dT] (SEQ | OSL21S | CAGUUGACAUUAAUGGUUUAA[dT][dT] |
| A | ID NO: 207) | | (SEQ ID NO: 581) |
| OSL22 | UAUUUAAACCAUUAAUGUCAA[dT][dT] (SEQ | OSL22S | UUGACAUUAAUGGUUUAAAUA[dT][dT] |
| A | ID NO: 208) | | (SEQ ID NO: 582) |
| OSL23 | UAGAUUUUAUUAUUAACCCAG[dT][dT] (SEQ | OSL23S | CUGGGUUAAUAAUAAAAUCUA[dT][dT] |
| A | ID NO: 209) | | (SEQ ID NO: 583) |
| OSL24 | AUAGAUUUUAUUAUUAACCCA[dT][dT] (SEQ | OSL24S | UGGGUUAAUAAUAAAAUCUAU[dT][dT] |
| A | ID NO: 210) | | (SEQ ID NO: 584) |
| OSL25 | AAAUUUACCAUAUCUAUGCGG[dT][dT] (SEQ | OSL25S | CCGCAUAGAUAUGGUAAAUUU[dT][dT] |
| A | ID NO: 211) | | (SEQ ID NO: 585) |
| OSL26 | AAGUUUUCAGUUAUAAGGGUA[dT][dT] (SEQ | OSL26S | UACCCUUAUAACUGAAAACUU[dT][dT] |
| A | ID NO: 212) | | (SEQ ID NO: 586) |
| OSL27 | AAUAAAUAACCAACAGUUGGG[dT][dT] (SEQ | OSL27S | CCCAACUGUUGGUUAUUUAUU[dT][dT] |
| A | ID NO: 213) | | (SEQ ID NO: 587) |
| OSL28 | AGAAAAAUAAAUAACCAACAG[dT][dT] (SEQ | OSL28S | CUGUUGGUUAUUUAUUUUUCU[dT][dT] |
| A | ID NO: 214) | | (SEQ ID NO: 588) |
| OSL29 | AUAUCAUAUCCAGAGUUACCC[dT][dT] (SEQ | OSL29S | GGGUAACUCUGGAUAUGAUAU[dT][dT] |
| A | ID NO: 215) | | (SEQ ID NO: 589) |
| OSL30 | AGUUUCAAUGUAAUCAAACCG[dT][dT] (SEQ | OSL30S | CGGUUUGAUUACAUUGAAACU[dT][dT] |
| A | ID NO: 216) | | (SEQ ID NO: 590) |
| OSL31 | UUACAGUUUCAAUGUAAUCAA[dT][dT] (SEQ | OSL31S | UUGAUUACAUUGAAACUGUAA[dT][dT] |
| A | ID NO: 217) | | (SEQ ID NO: 591) |
| OSL32 | AUAAGUUACAGUUUCAAUGUA[dT][dT] (SEQ | OSL32S | UACAUUGAAACUGUAACUUAU[dT][dT] |
| A | ID NO: 218) | | (SEQ ID NO: 592) |
| OSL33 | UCUUUACACGGAUUGGUAGCA[dT][dT] (SEQ | OSL33S | UGCUACCAAUCCGUGUAAAGA[dT][dT] |
| A | ID NO: 219) | | (SEQ ID NO: 593) |
| OSL34 | AAAAUCAAUCCCGACAAAGAA[dT][dT] (SEQ | OSL34S | UUCUUUGUCGGGAUUGAUUUU[dT][dT] |
| A | ID NO: 220) | | (SEQ ID NO: 594) |
| OSL35 | UCUGAAAAAAAGAUAGUGCUG[dT][dT] (SEQ | OSL35S | CAGCACUAUCUUUUUUUCAGA[dT][dT] |
| A | ID NO: 221) | | (SEQ ID NO: 595) |
| OSL36 | AUCUGAAAAAAAGAUAGUGCU[dT][dT] (SEQ | OSL36S | AGCACUAUCUUUUUUUCAGAU[dT][dT] |
| A | ID NO: 222) | | (SEQ ID NO: 596) |
| OSL37 | AAAAAUCAUGUGUUUUGACAU[dT][dT] (SEQ | OSL37S | AUGUCAAAACACAUGAUUUUU[dT][dT] |
| A | ID NO: 223) | | (SEQ ID NO: 597) |
| OSL38 | UUUGCUUAAAAAUCAUGUGUU[dT][dT] (SEQ | OSL38S | AACACAUGAUUUUUAAGCAAA[dT][dT] |
| A | ID NO: 224) | | (SEQ ID NO: 598) |
| OSL39 | AACUUUCAACAUUUUCCACCC[dT][dT] (SEQ | OSL39S | GGGUGGAAAAUGUUGAAAGUU[dT][dT] |
| A | ID NO: 225) | | (SEQ ID NO: 599) |
| OSL40 | UUGAAAUCCAAUCAAAAGCCA[dT][dT] (SEQ | OSL40S | UGGCUUUUGAUUGGAUUUCAA[dT][dT] |
| A | ID NO: 226) | | (SEQ ID NO: 600) |
| OSL41 | UUUGAAAUCCAAUCAAAAGCC[dT][dT] (SEQ | OSL41S | GGCUUUUGAUUGGAUUUCAAA[dT][dT] |
| A | ID NO: 227) | | (SEQ ID NO: 601) |
| OSL42 | UAGAGAUUCUUUGAAAUCCAA[dT][dT] (SEQ | OSL42S | UUGGAUUUCAAAGAAUCUCUA[dT][dT] |
| A | ID NO: 228) | | (SEQ ID NO: 602) |
| OSL43 | AUAGAGAUUCUUUGAAAUCCA[dT][dT] (SEQ | OSL43S | UGGAUUUCAAAGAAUCUCUAU[dT][dT] |
| A | ID NO: 229) | | (SEQ ID NO: 603) |
| OSL44 | UUUAAAUACUGAACUACUGUG[dT][dT] (SEQ | OSL44S | CACAGUAGUUCAGUAUUUAAA[dT][dT] |
| A | ID NO: 230) | | (SEQ ID NO: 604) |
| OSL45 | UAUUUAAAUACUGAACUACUG[dT][dT] (SEQ | OSL45S | CAGUAGUUCAGUAUUUAAAUA[dT][dT] |
| A | ID NO: 231) | | (SEQ ID NO: 605) |
| OSL46 | AUAGAUACCCGGCAAAAGGAU[dT][dT] (SEQ | OSL46S | AUCCUUUUGCCGGGUAUCUAU[dT][dT] |
| A | ID NO: 232) | | (SEQ ID NO: 606) |
| OSL47 | AAGAGUAGUGUUUAUUACAGG[dT][dT] (SEQ | OSL47S | CCCCUGUAAUAAACACUACUC[dT][dT] |
| A | ID NO: 233) | | (SEQ ID NO: 607) |
| OSL48 | AUCAAAAUAGGCAUCUACCCA[dT][dT] (SEQ | OSL48S | UGGGUAGAUGCCUAUUUUGAU[dT][dT] |
| A | ID NO: 234) | | (SEQ ID NO: 608) |
| OSL49 | UCAAUUUUAUCAAAAUAGGCA[dT][dT] (SEQ | OSL49S | UGCCUAUUUUGAUAAAAUUGA[dT][dT] |
| A | ID NO: 235) | | (SEQ ID NO: 609) |
| OSL50 | UAAAUGCUCUCCAAAGAUGGC[dT][dT] (SEQ | OSL50S | GCCAUCUUUGGAGAGCAUUUA[dT][dT] |
| A | ID NO: 236) | | (SEQ ID NO: 610) |
| OSL51 | UCAAAUGCAGUAUGUAAGCAA[dT][dT] (SEQ | OSL51S | UUGCUUACAUACUGCAUUUGA[dT][dT] |
| A | ID NO: 237) | | (SEQ ID NO: 611) |
| OSL52 | UUCAAAUGCAGUAUGUAAGCA[dT][dT] (SEQ | OSL52S | UGCUUACAUACUGCAUUUGAA[dT][dT] |
| A | ID NO: 238) | | (SEQ ID NO: 612) |
| OSL53 | UGAUUACAGGCGUUAGAACCA[dT][dT] (SEQ | OSL53S | UGGUUCUAACGCCUGUAAUCA[dT][dT] |
| A | ID NO: 239) | | (SEQ ID NO: 613) |
| OSL54 | UGUUAUCAUGACAAUCAUCGA[dT][dT] (SEQ | OSL54S | UCGAUGAUUGUCAUGAUAACA[dT][dT] |
| A | ID NO: 240) | | (SEQ ID NO: 614) |
| OSL55 | UUAUCACAGGUGUAUUGGGUG[dT][dT] (SEQ | OSL55S | CACCCAAUACACCUGUGAUAA[dT][dT] |
| A | ID NO: 241) | | (SEQ ID NO: 615) |
| OSL56 | AUUAUCACAGGUGUAUUGGGU[dT][dT] (SEQ | OSL56S | ACCCAAUACACCUGUGAUAAU[dT][dT] |
| A | ID NO: 242) | | (SEQ ID NO: 616) |
| OSL57 | AGUUCUUUGAGAUACACUGGU[dT][dT] (SEQ | OSL57S | ACCAGUGUAUCUCAAAGAACU[dT][dT] |
| A | ID NO: 243) | | (SEQ ID NO: 617) |
| OSL58 | UCGAAUUGCAGUUCUUUUCAU[dT][dT] (SEQ | OSL58S | AUGAAAAGAACUGCAAUUCGA[dT][dT] |
| A | ID NO: 244) | | (SEQ ID NO: 618) |
| OSL59 | UCAAUACAUCGAUGAUUGGGG[dT][dT] (SEQ | OSL59S | CCCCAAUCAUCGAUGUAUUGA[dT][dT] |
| A | ID NO: 245) | | (SEQ ID NO: 619) |
| OSL60 | AACGAUAGGUCAAUACAUCGA[dT][dT] (SEQ | OSL60S | UCGAUGUAUUGACCUAUCGUU[dT][dT] |
| A | ID NO: 246) | | (SEQ ID NO: 620) |
| OSL61 | ACAAACGAUAGGUCAAUACAU[dT][dT] (SEQ | OSL61S | AUGUAUUGACCUAUCGUUUGU[dT][dT] |
| A | ID NO: 247) | | (SEQ ID NO: 621) |
| OSL62 | UCAAAAACACCAUCACAACGA[dT][dT] (SEQ ID | OSL62S | UCGUUGUGAUGGUGUUUUUGA[dT][dT] |
| A | NO: 248) | | (SEQ ID NO: 622) |
| OSL63 | UCACAUUCCCAGAAGUUCGGG[dT][dT] (SEQ | OSL63S | CCCGAACUUCUGGGAAUGUGA[dT][dT] |
| A | ID NO: 249) | | (SEQ ID NO: 623) |
| OSL64 | AUCACAUUCCCAGAAGUUCGG[dT][dT] (SEQ | OSL64S | CCGAACUUCUGGGAAUGUGAU[dT][dT] |
| A | ID NO: 250) | | (SEQ ID NO: 624) |
| OSL65 | UGAUGAAGGGCAAGUCUUGGG[dT][dT] (SEQ | OSL65S | CCCAAGACUUGCCCUUCAUCA[dT][dT] |
| A | ID NO: 251) | | (SEQ ID NO: 625) |
| OSL66 | AGAAUCAUUGGCAAGUAAGAA[dT][dT] (SEQ | OSL66S | UUCUUACUUGCCAAUGAUUCU[dT][dT] |
| A | ID NO: 252) | | (SEQ ID NO: 626) |
| OSL67 | UAUCACAUUCAUCUAUGUCUU[dT][dT] (SEQ | OSL67S | AAGACAUAGAUGAAUGUGAUA[dT][dT] |
| A | ID NO: 253) | | (SEQ ID NO: 627) |
| OSL68 | AAUAUCACAUUCAUCUAUGUC[dT][dT] (SEQ | OSL68S | GACAUAGAUGAAUGUGAUAUU[dT][dT] |
| A | ID NO: 254) | | (SEQ ID NO: 628) |
| OSL69 | AACAUGUAGCCUGUAUCACAC[dT][dT] (SEQ | OSL69S | GUGUGAUACAGGCUACAUGUU[dT][dT] |
| A | ID NO: 255) | | (SEQ ID NO: 629) |
| OSL70 | UCACUUUCUAACAUGUAGCCU[dT][dT] (SEQ | OSL70S | AGGCUACAUGUUAGAAAGUGA[dT][dT] |
| A | ID NO: 256) | | (SEQ ID NO: 630) |
| OSL71 | AUCACUUUCUAACAUGUAGCC[dT][dT] (SEQ | OSL71S | GGCUACAUGUUAGAAAGUGAU[dT][dT] |
| A | ID NO: 257) | | (SEQ ID NO: 631) |
| OSL72 | AAUGUAAGAACCAUUCUCGAC[dT][dT] (SEQ | OSL72S | GUCGAGAAUGGUUCUUACAUU[dT][dT] |
| A | ID NO: 258) | | (SEQ ID NO: 632) |
| OSL73 | UACAAUGUAAGAACCAUUCUC[dT][dT] (SEQ | OSL73S | GAGAAUGGUUCUUACAUUGUA[dT][dT] |
| A | ID NO: 259) | | (SEQ ID NO: 633) |
| OSL74 | AAAAUCAACAGCUACAAUGUA[dT][dT] (SEQ | OSL74S | UACAUUGUAGCUGUUGAUUUU[dT][dT] |
| A | ID NO: 260) | | (SEQ ID NO: 634) |
| OSL75 | AUUGAAUCAAAAUCAACAGCU[dT][dT] (SEQ | OSL75S | AGCUGUUGAUUUUGAUUCAAU[dT][dT] |
| A | ID NO: 261) | | (SEQ ID NO: 635) |
| OSL76 | UAAUUGAAUCAAAAUCAACAG[dT][dT] (SEQ | OSL76S | CUGUUGAUUUUGAUUCAAUUA[dT][dT] |
| A | ID NO: 262) | | (SEQ ID NO: 636) |
| OSL77 | AAGAUACGACCACUAAUUGAA[dT][dT] (SEQ | OSL77S | UUCAAUUAGUGGUCGUAUCUU[dT][dT] |
| A | ID NO: 263) | | (SEQ ID NO: 637) |
| OSL78 | AGUUUCAGUCAAGAUGAUGCU[dT][dT] (SEQ | OSL78S | AGCAUCAUCUUGACUGAAACU[dT][dT] |
| A | ID NO: 264) | | (SEQ ID NO: 638) |
| OSL79 | AAUAGUUUCAGUCAAGAUGAU[dT][dT] (SEQ | OSL79S | AUCAUCUUGACUGAAACUAUU[dT][dT] |
| A | ID NO: 265) | | (SEQ ID NO: 639) |
| OSL80 | UAUUGCAAUAGUUUCAGUCAA[dT][dT] (SEQ | OSL80S | UUGACUGAAACUAUUGCAAUA[dT][dT] |
| A | ID NO: 266) | | (SEQ ID NO: 640) |
| OSL81 | UCUAUUGCAAUAGUUUCAGUC[dT][dT] (SEQ | OSL81S | GACUGAAACUAUUGCAAUAGA[dT][dT] |
| A | ID NO: 267) | | (SEQ ID NO: 641) |
| OSL82 | AAUCUAUUGCAAUAGUUUCAG[dT][dT] (SEQ | OSL82S | CUGAAACUAUUGCAAUAGAUU[dT][dT] |
| A | ID NO: 268) | | (SEQ ID NO: 642) |
| OSL83 | AUUUUGGAGACUUCAAUUGUU[dT][dT] (SEQ | OSL83S | AACAAUUGAAGUCUCCAAAAU[dT][dT] |
| A | ID NO: 269) | | (SEQ ID NO: 643) |
| OSL84 | UUAGGUUUUUACUAAUCAGCA[dT][dT] (SEQ | OSL84S | UGCUGAUUAGUAAAAACCUAA[dT][dT] |
| A | ID NO: 270) | | (SEQ ID NO: 644) |
| OSL85 | UCAUUCUGGGAUCUAAUGCUA[dT][dT] (SEQ | OSL85S | UAGCAUUAGAUCCCAGAAUGA[dT][dT] |
| A | ID NO: 271) | | (SEQ ID NO: 645) |
| OSL86 | UUCAUUCUGGGAUCUAAUGCU[dT][dT] (SEQ | OSL86S | AGCAUUAGAUCCCAGAAUGAA[dT][dT] |
| A | ID NO: 272) | | (SEQ ID NO: 646) |
| OSL87 | AGUAGAUGCUCAUUCAUUCUG[dT][dT] (SEQ | OSL87S | CAGAAUGAAUGAGCAUCUACU[dT][dT] |
| A | ID NO: 273) | | (SEQ ID NO: 647) |
| OSL88 | AUUAUAAUCACAAAAGUCCAU[dT][dT] (SEQ | OSL88S | AUGGACUUUUGUGAUUAUAAU[dT][dT] |
| A | ID NO: 274) | | (SEQ ID NO: 648) |
| OSL89 | UCCAUUAUAAUCACAAAAGUC[dT][dT] (SEQ | OSL89S | GACUUUUGUGAUUAUAAUGGA[dT][dT] |
| A | ID NO: 275) | | (SEQ ID NO: 649) |
| OSL90 | UGCCGUAUAAUCAAAUCAC[dT][dT] (SEQ ID | OSL90S | GUGUGAUUUGAUUAUACGGCA[dT][dT] |
| A | NO: 276) | | (SEQ ID NO: 650) |
| OSL91 | AUAUUAUACAUUACAACUGAC[dT][dT] (SEQ | OSL91S | GUCAGUUGUAAUGUAUAAUAU[dT][dT] |
| A | ID NO: 277) | | (SEQ ID NO: 651) |
| OSL92 | AUUGAAUAUUAUACAUUACAA[dT][dT] (SEQ | OSL92S | UUGUAAUGUAUAAUAUUCAAU[dT][dT] |
| A | ID NO: 278) | | (SEQ ID NO: 652) |
| OSL93 | AAUUUGGUUGUUUCGAAGGAU[dT][dT] (SEQ | OSL93S | AUCCUUCGAAACAACCAAAUU[dT][dT] |
| A | ID NO: 279) | | (SEQ ID NO: 653) |
| OSL94 | ACGGAAUUUGGUUGUUUCGAA[dT][dT] (SEQ | OSL94S | UUCGAAACAACCAAAUUCCGU[dT][dT] |
| A | ID NO: 280) | | (SEQ ID NO: 654) |
| OSL95 | UUACAGUUAUUAAGAAAGGUU[dT][dT] (SEQ | OSL95S | AACCUUUCUUAAUAACUGUAA[dT][dT] |
| A | ID NO: 281) | | (SEQ ID NO: 655) |
| OSL96 | UCCAAAAAUUAUAUGUUGCCU[dT][dT] (SEQ | OSL96S | AGGCAACAUAUAAUUUUUGGA[dT][dT] |
| A | ID NO: 282) | | (SEQ ID NO: 656) |
| OSL97 | UUCCAAAAAUUAUAUGUUGCC[dT][dT] (SEQ | OSL97S | GGCAACAUAUAAUUUUUGGAA[dT[dT] |
| A | ID NO: 283) | | (SEQ ID NO: 657) |
| OSL98 | UCUAAACCAUUCUGUAUCCCU[dT][dT] (SEQ | OSL98S | AGGGAUACAGAAUGGUUUAGA[dT][dT] |
| A | ID NO: 284) | | (SEQ ID NO: 658) |
| OSL99 | AUCUAAACCAUUCUGUAUCCC[dT][dT] (SEQ | OSL99S | GGGAUACAGAAUGGUUUAGAU[dT][dT] |
| A | ID NO: 285) | | (SEQ ID NO: 659) |
| OSL100 | UUCAACAUCUAAACCAUUCUG[dT][dT] (SEQ | OSL100 | CAGAAUGGUUUAGAUGUUGAA[dT[dT] |
| A | ID NO: 286) | S | (SEQ ID NO: 660) |
| OSL101 | AUUUUCAACCCAAUAGAUGUA[dT][dT] (SEQ | OSL101 | UACAUCUAUUGGGUUGAAAAU[dT][dT] |
| A | ID NO: 287) | S | (SEQ ID NO: 661) |
| OSL102 | UAUAGAAGCAAAUACUGUCCU[dT][dT] (SEQ | OSL102 | AGGACAGUAUUUGCUUCUAUA[dT][dT] |
| A | ID NO: 288) | S | (SEQ ID NO: 662) |
| OSL103 | UAGAUAUAGAAGCAAAUACUG[dT][dT] (SEQ | OSL103 | CAGUAUUUGCUUCUAUAUCUA[dT][dT] |
| A | ID NO: 289) | S | (SEQ ID NO: 663) |
| OSL104 | UAAGGCCAGGUUCAUAGAAGG[dT][dT] (SEQ | OSL104 | CCUUCUAUGAACCUGGCCU[dT][dT] (SEQ |
| A | ID NO: 290) | S | ID NO: 664) |
| OSL105 | UCUUGAAAUCCAAUCUAAGGC[dT][dT] (SEQ | OSL105 | GCCUUAGAUUGGAUUUCAAGA[dT][dT] |
| A | ID NO: 291) | S | (SEQ ID NO: 665) |
| OSL106 | AUAAAGGUUUCUUGAAAUCCA[dT][dT] (SEQ | OSL106 | UGGAUUUCAAGAAACCUUUAU[dT][dT] |
| A | ID NO: 292) | S | (SEQ ID NO: 666) |
| OSL107 | UCAAAACCUCGAUUGACUGAG[dT][dT] (SEQ | OSL107 | CUCAGUCAAUCGAGGUUUUGA[dT][dT] |
| A | ID NO: 293) | S | (SEQ ID NO: 667) |
| OSL108 | UUCUGUAUCUGAUAUCUCCGU[dT][dT] (SEQ | OSL108 | ACGGAGAUAUCAGAUACAGAA[dT][dT] |
| A | ID NO: 294) | S | (SEQ ID NO: 668) |
| OSL109 | UUUCUGUAUCUGAUAUCUCCG[dT][dT] (SEQ | OSL109 | CGGAGAUAUCAGAUACAGAAA[dT][dT] |
| A | ID NO: 295) | S | (SEQ ID NO: 669) |
| OSL110 | UUUUUCUGUAUCUGAUAUCUC[dT][dT] (SEQ | OSL110 | GAGAUAUCAGAUACAGAAAAA[dT][dT] |
| A | ID NO: 296) | S | (SEQ ID NO: 670) |
| OSL111 | AUCAAUGUUUUUCUGUAUCUG[dT][dT] (SEQ | OSL111 | CAGAUACAGAAAAACAUUGAU[dT][dT] |
| A | ID NO: 297) | S | (SEQ ID NO: 671) |
| OSL112 | AUAAAGGAAAGAAUCAUGGAC[dT][dT] (SEQ | OSL112 | GUCCAUGAUUCUUUCCUUUAU[dT][dT] |
| A | ID NO: 298) | S | (SEQ ID NO: 672) |
| OSL113 | AAUAAAGGAAAGAAUCAUGGA[dT][dT] (SEQ | OSL113 | UCCAUGAUUCUUUCCUUUAUU[dT][dT] |
| A | ID NO: 299) | S | (SEQ ID NO: 673) |
| OSL114 | UCAGUAUAAUAAAGGAAAGAA[dT][dT] (SEQ | OSL114 | UUCUUUCCUUUAUUAUACUGA[dT][dT] |
| A | ID NO: 300) | S | (SEQ ID NO: 674) |
| OSL115 | UUUCAAUGACCUCAUACUGUU[dT][dT] (SEQ | OSL115 | AACAGUAUGAGGUCAUUGAAA[dT][dT] |
| A | ID NO: 301) | S | (SEQ ID NO: 675) |
| OSL116 | AUUUGGAACAUUAUCUCUCAA[dT][dT] (SEQ | OSL116 | UUGAGAGAUAAUGUUCCAAAU[dT][dT] |
| A | ID NO: 302) | S | (SEQ ID NO: 676) |
| OSL117 | AGAUUUGGAACAUUAUCUCUC[dT][dT] (SEQ | OSL117 | GAGAGAUAAUGUUCCAAAUCU[dT][dT] |
| A | ID NO: 303) | S | (SEQ ID NO: 677) |
| OSL118 | UCAGAUUUGGAACAUUAUCUC[dT][dT] (SEQ | OSL118 | GAGAUAAUGUUCCAAAUCUGA[dT][dT] |
| A | ID NO: 304) | S | (SEQ ID NO: 678) |
| OSL119 | UUGCUACAGCCAUUUGAGG[dT][dT] (SEQ ID | OSL119 | CCUCAAAUGGCUGUAGCAA[dT][dT] (SEQ |
| A | NO: 305) | S | ID NO: 679) |
| OSL120 | AUGAAAGAGUUAUAUGGAGAG[dT][dT] (SEQ | OSL120 | CUCUCCAUAUAACUCUUUCAU[dT][dT] |
| A | ID NO: 306) | S | (SEQ ID NO: 680) |
| OSL121 | ACAAUGAAAGAGUUAUAUGGA[dT][dT] (SEQ | OSL121 | UCCAUAUAACUCUUUCAUUGU[dT][dT] |
| A | ID NO: 307) | S | (SEQ ID NO: 681) |
| OSL122 | UGAAACAACAAUGAAAGAGUU[dT][dT] (SEQ | OSL122 | AACUCUUUCAUUGUUGUUUCA[dT][dT] |
| A | ID NO: 308) | S | (SEQ ID NO: 682) |
| OSL123 | AUUGAAACAACAAUGAAAGAG[dT][dT] (SEQ | OSL123 | CUCUUUCAUUGUUGUUUCAAU[dT][dT] |
| A | ID NO: 309) | S | (SEQ ID NO: 683) |
| OSL124 | AGCUAAAGCCUCUGAUUGCAG[dT][dT] (SEQ | OSL124 | CUGCAAUCAGAGGCUUUAGCU[dT][dT] |
| A | ID NO: 310) | S | (SEQ ID NO: 684) |
| OSL125 | AAGCUAAAGCCUCUGAUUGCA[dT][dT] (SEQ | OSL125 | UGCAAUCAGAGGCUUUAGCUU[dT][dT] |
| A | ID NO: 311) | S | (SEQ ID NO: 685) |
| OSL126 | ACAAUUCCAAGCUAAAGCCUC[dT][dT] (SEQ | OSL126 | GAGGCUUUAGCUUGGAAUUGU[dT][dT] |
| A | ID NO: 312) | S | (SEQ ID NO: 686) |
| OSL127 | UGACAAUUCCAAGCUAAAGCC[dT][dT] (SEQ | OSL127 | GGCUUUAGCUUGGAAUUGUCA[dT][dT] |
| A | ID NO: 313) | S | (SEQ ID NO: 687) |
| OSL128 | UGAAUGAUCUGACAAUUCCAA[dT][dT] (SEQ | OSL128 | UUGGAAUUGUCAGAUCAUUCA[dT][dT] |
| A | ID NO: 314) | S | (SEQ ID NO: 688) |
| OSL129 | AUGUUCAUCAGAGAAGAUCCA[dT][dT] (SEQ | OSL129 | UGGAUCUUCUCUGAUGAACAU[dT][dT] |
| A | ID NO: 315) | S | (SEQ ID NO: 689) |
| OSL130 | UAUUCCAUGUGUCACAAUGUU[dT][dT] (SEQ | OSL130 | AACAUUGUGACACAUGGAAUA[dT][dT] |
| A | ID NO: 316) | S | (SEQ ID NO: 690) |
| OSL131 | ACUUCUAUCAGUGUUUCAGAA[dT][dT] (SEQ | OSL131 | UUCUGAAACACUGAUAGAAGU[dT][dT] |
| A | ID NO: 317) | S | (SEQ ID NO: 691) |
| OSL132 | UAUUGAUCCGCAGAACUUCUA[dT][dT] (SEQ | OSL132 | UAGAAGUUCUGCGGAUCAAUA[dT][dT] |
| A | ID NO: 318) | S | (SEQ ID NO: 692) |
| OSL133 | UGUUCUUGGGAUCUACAACAA[dT][dT] (SEQ | OSL133 | UUGUUGUAGAUCCCAAGAACA[dT][dT] |
| A | ID NO: 319) | S | (SEQ ID NO: 693) |
| OSL134 | AAAGAACGCUCAAUCUUUGGU[dT][dT] (SEQ | OSL134 | ACCAAAGAUUGAGCGUUCUUU[dT][dT] |
| A | ID NO: 320) | S | (SEQ ID NO: 694) |
| OSL135 | UAAACGUAGCCAUCACUUCGG[dT][dT] (SEQ | OSL135 | CCGAAGUGAUGGCUACGUUUA[dT][dT] |
| A | ID NO: 321) | S | (SEQ ID NO: 695) |
| OSL136 | AUCUAAAGAAUCAUCAACCCA[dT][dT] (SEQ | OSL136 | UGGGUUGAUGAUUCUUUAGAU[dT][dT] |
| A | ID NO: 322) | S | (SEQ ID NO: 696) |
| OSL137 | UUAUAUCUAAAGAAUCAUCAA[dT][dT] (SEQ | OSL137 | UUGAUGAUUCUUUAGAUAUAA[dT][dT] |
| A | ID NO: 323) | S | (SEQ ID NO: 697) |
| OSL138 | AUAGAAUUUUCAAAAACAGUG[dT][dT] (SEQ | OSL138 | CACUGUUUUUGAAAAUUCUAU[dT][dT] |
| A | ID NO: 324) | S | (SEQ ID NO: 698) |
| OSL139 | UGAUAGAAUUUUCAAAAACAG[dT][dT] (SEQ | OSL139 | CUGUUUUUGAAAAUUCUAUCA[dT][dT] |
| A | ID NO: 325) | S | (SEQ ID NO: 699) |
| OSL140 | UUUCAAAUUCCUAUCUACCCA[dT][dT] (SEQ | OSL140 | UGGGUAGAUAGGAAUUUGAAA[dT][dT] |
| A | ID NO: 326) | S | (SEQ ID NO: 700) |
| OSL141 | UUUUCAAAUUCCUAUCUACCC[dT][dT] (SEQ | OSL141 | GGGUAGAUAGGAAUUUGAAAA[dT][dT] |
| A | ID NO: 327) | S | (SEQ ID NO: 701) |
| OSL142 | AUAUUGUCUCUUAUCACUGUG[dT][dT] (SEQ | OSL142 | CACAGUGAUAAGAGACAAUAU[dT][dT] |
| A | ID NO: 328) | S | (SEQ ID NO: 702) |
| OSL143 | UGAUAUUGUCUCUUAUCACUG[dT][dT] (SEQ | OSL143 | CAGUGAUAAGAGACAAUAUCA[dT][dT] |
| A | ID NO: 329) | S | (SEQ ID NO: 703) |
| OSL144 | UGAAAUGGCACAAUUCUUGCC[dT][dT] (SEQ | OSL144 | GGCAAGAAUUGUGCCAUUUCA[dT][dT] |
| A | ID NO: 330) | S | (SEQ ID NO: 704) |
| OSL145 | UGUUGAAAUGGCACAAUUCUU[dT][dT] (SEQ | OSL145 | AAGAAUUGUGCCAUUUCAACA[dT][dT] |
| A | ID NO: 331) | S | (SEQ ID NO: 705) |
| OSL146 | AAUUUUCUGUUGAAAUGGCAC[dT][dT] (SEQ | OSL146 | GUGCCAUUUCAACAGAAAAUU[dT][dT] |
| A | ID NO: 332) | S | (SEQ ID NO: 706) |
| OSL147 | AAAUUUUCUGUUGAAAUGGCA[dT][dT] (SEQ | OSL147 | UGCCAUUUCAACAGAAAAUUU[dT][dT] |
| A | ID NO: 333) | S | (SEQ ID NO: 707) |
| OSL148 | AUUAGACAAGGCAAAGAUGAG[dT][dT] (SEQ | OSL148 | CUCAUCUUUGCCUUGUCUAAU[dT][dT] |
| A | ID NO: 334) | S | (SEQ ID NO: 708) |
| OSL149 | ACAUUUAUUGUUUGGAAAGGU[dT][dT] (SEQ | OSL149 | ACCUUUCCAAACAAUAAAUGU[dT][dT] |
| A | ID NO: 335) | S | (SEQ ID NO: 709) |
| OSL150 | AUCACUUACACUGUCAUAGUC[dT][dT] (SEQ | OSL150 | GACUAUGACAGUGUAAGUGAU[dT][dT] |
| A | ID NO: 336) | S | (SEQ ID NO: 710) |
| OSL151 | UAGAUUCUAUCACUUACACUG[dT][dT] (SEQ | OSL151 | CAGUGUAAGUGAUAGAAUCUA[dT][dT] |
| A | ID NO: 337) | S | (SEQ ID NO: 711) |
| OSL152 | AGUAGAUUCUAUCACUUACAC[dT][dT] (SEQ | OSL152 | GUGUAAGUGAUAGAAUCUACU[dT][dT] |
| A | ID NO: 338) | S | (SEQ ID NO: 712) |
| OSL153 | UUUUGUGUGAAGUAGAUUCUA[dT][dT] (SEQ | OSL153 | UAGAAUCUACUUCACACAAAA[dT][dT] |
| A | ID NO: 339) | S | (SEQ ID NO: 713) |
| OSL154 | UAAAUUUUGUGUGAAGUAGAU[dT][dT] (SEQ | OSL154 | AUCUACUUCACACAAAAUUUA[dT][dT] |
| A | ID NO: 340) | S | (SEQ ID NO: 714) |
| OSL155 | UCUAGUAAUCCAGUCAAAGGC[dT][dT] (SEQ | OSL155 | GCCUUUGACUGGAUUACUAGA[dT][dT] |
| A | ID NO: 341) | S | (SEQ ID NO: 715) |
| OSL156 | AAUUCUUCUAGUAAUCCAGUC[dT][dT] (SEQ | OSL156 | GACUGGAUUACUAGAAGAAUU[dT][dT] |
| A | ID NO: 342) | S | (SEQ ID NO: 716) |
| OSL157 | UAAAUUCUUCUAGUAAUCCAG[dT][dT] (SEQ | OSL157 | CUGGAUUACUAGAAGAAUUUA[dT][dT] |
| A | ID NO: 343) | S | (SEQ ID NO: 717) |
| OSL158 | AUAAAUUCUUCUAGUAAUCCA[dT][dT] (SEQ | OSL158 | UGGAUUACUAGAAGAAUUUAU[dT][dT] |
| A | ID NO: 344) | S | (SEQ ID NO: 718) |
| OSL159 | AUAUACUGGCCAUAGAGAGUC[dT][dT] (SEQ | OSL159 | GACUCUCUAUGGCCAGUAUAU[dT][dT] |
| A | ID NO: 345) | S | (SEQ ID NO: 719) |
| OSL160 | UUCUUUGUGUGUACAAGUCAG[dT][dT] (SEQ | OSL160 | CUGACUUGUACACACAAAGAA[dT][dT] |
| A | ID NO: 346) | S | (SEQ ID NO: 720) |
| OSL161 | AAUUCUUUGUGUGUACAAGUC[dT][dT] (SEQ | OSL161 | GACUUGUACACACAAAGAAUU[dT][dT] |
| A | ID NO: 347) | S | (SEQ ID NO: 721) |
| OSL162 | UCGGUAAAUUCUUUGUGUGUA[dT][dT] (SEQ | OSL162 | UACACACAAAGAAUUUACCGA[dT][dT] |
| A | ID NO: 348) | S | (SEQ ID NO: 722) |
| OSL163 | UGUUACACUGUUGUUUCUGGU[dT][dT] (SEQ | OSL163 | ACCAGAAACAACAGUGUAACA[dT][dT] |
| A | ID NO: 349) | S | (SEQ ID NO: 723) |
| OSL164 | AUUGUUACACUGUUGUUUCUG[dT][dT] (SEQ | OSL164 | CAGAAACAACAGUGUAACAAU[dT][dT] |
| A | ID NO: 350) | S | (SEQ ID NO: 724) |
| OSL165 | AAACUGUUCACAAGGAUUGUU[dT][dT] (SEQ | OSL165 | AACAAUCCUUGUGAACAGUUU[dT][dT] |
| A | ID NO: 351) | S | (SEQ ID NO: 725) |
| OSL166 | ACAUCGUUCACCAUUGUCCAC[dT][dT] (SEQ | OSL166 | GUGGACAAUGGUGAACGAUGU[dT][dT] |
| A | ID NO: 352) | S | (SEQ ID NO: 726) |
| OSL167 | UGUUAUUGCACAUAAACUCCG[dT][dT] (SEQ | OSL167 | CGGAGUUUAUGUGCAAUAACA[dT][dT] |
| A | ID NO: 353) | S | (SEQ ID NO: 727) |
| OSL168 | UCUGUUAUUGCACAUAAACUC[dT][dT] (SEQ | OSL168 | GAGUUUAUGUGCAAUAACAGA[dT][dT] |
| A | ID NO: 354) | S | (SEQ ID NO: 728) |
| OSL169 | UUAUGACAUUUUGUGUAUCCA[dT][dT] (SEQ | OSL169 | UGGAUACACAAAAUGUCAUAA[dT][dT] |
| A | ID NO: 355) | S | (SEQ ID NO: 729) |
| OSL170 | UGAAUUAUGACAUUUUGUGUA[dT][dT] (SEQ | OSL170 | UACACAAAAUGUCAUAAUUCA[dT][dT] |
| A | ID NO: 356) | S | (SEQ ID NO: 730) |
| OSL171 | UUUGAAUUAUGACAUUUUGUG[dT][dT] (SEQ | OSL171 | CACAAAAUGUCAUAAUUCAAA[dT][dT] |
| A | ID NO: 357) | S | (SEQ ID NO: 731) |
| OSL172 | UACAAAUAUUUGAAUUAUGAC[dT][dT] (SEQ | OSL172 | GUCAUAAUUCAAAUAUUUGUA[dT][dT] |
| A | ID NO: 358) | S | (SEQ ID NO: 732) |
| OSL173 | AAAUAAACGCGAGGAAUACAA[dT][dT] (SEQ | OSL173 | UUGUAUUCCUCGCGUUUAUUU[dT][dT] |
| A | ID NO: 359) | S | (SEQ ID NO: 733) |
| OSL174 | AAUAAGUAGGGUUUUCAUCAC[dT][dT] (SEQ | OSL174 | GUGAUGAAAACCCUACUUAUU[dT][dT] |
| A | ID NO: 360) | S | (SEQ ID NO: 734) |
| OSL175 | UCACAAUACCAAUGUUGAGGA[dT][dT] (SEQ | OSL175 | UCCUCAACAUUGGUAUUGUGA[dT][dT] |
| A | ID NO: 361) | S | (SEQ ID NO: 735) |
| OSL176 | UGUUUCUUGAUCACAAUACCA[dT][dT] (SEQ | OSL176 | UGGUAUUGUGAUCAAGAAACA[dT][dT] |
| A | ID NO: 362) | S | (SEQ ID NO: 736) |
| OSL177 | AACAAUCUGUUUCUUGAUCAC[dT][dT] (SEQ | OSL177 | GUGAUCAAGAAACAGAUUGUU[dT][dT] |
| A | ID NO: 363) | S | (SEQ ID NO: 737) |
| OSL178 | UUUACACAGAGAAACUCGGAA[dT][dT] (SEQ | OSL178 | UUCCGAGUUUCUCUGUGUAAA[dT][dT] |
| A | ID NO: 364) | S | (SEQ ID NO: 738) |
| OSL179 | AUUUACACAGAGAAACUCGGA[dT][dT] (SEQ | OSL179 | UCCGAGUUUCUCUGUGUAAAU[dT][dT] |
| A | ID NO: 365) | S | (SEQ ID NO: 739) |
| OSL180 | UUCUGAUUCUCAUCGUAGCCG[dT][dT] (SEQ | OSL180 | CGGCUACGAUGAGAAUCAGAA[dT][dT] |
| A | ID NO: 366) | S | (SEQ ID NO: 740) |
| OSL181 | AUUUUCAGAGCAAGUUCUCCU[dT][dT] (SEQ | OSL181 | AGGAGAACUUGCUCUGAAAAU[dT][dT] |
| A | ID NO: 367) | S | (SEQ ID NO: 741) |
| OSL182 | AUAUCUUUGGGAUACACAGUC[dT][dT] (SEQ | OSL182 | GACUGUGUAUCCCAAAGAUAU[dT][dT] |
| A | ID NO: 368) | S | (SEQ ID NO: 742) |
| OSL183 | AGGUAAACUGAUUCUGUUGGC[dT][dT] (SEQ | OSL183 | GCCAACAGAAUCAGUUUACCU[dT][dT] |
| A | ID NO: 369) | S | (SEQ ID NO: 743) |
| OSL184 | AUAGAAACUGGUUAAGGUGUC[dT][dT] (SEQ | OSL184 | GACACCUUAACCAGUUUCUAU[dT][dT] |
| A | ID NO: 370) | S | (SEQ ID NO: 744) |
| OSL185 | ACAAUAGAAACUGGUUAAGGU[dT][dT] (SEQ | OSL185 | ACCUUAACCAGUUUCUAUUGU[dT][dT] |
| A | ID NO: 371) | S | (SEQ ID NO: 745) |
| OSL186 | UCAUCAAUAUCAACACAAGUC[dT][dT] (SEQ | OSL186 | GACUUGUGUUGAUAUUGAUGA[dT][dT] |
| A | ID NO: 372) | S | (SEQ ID NO: 746) |
| OSL187 | AGAUGUAGGAGCCUAUUACAU[dT][dT] (SEQ | OSL187 | AUGUAAUAGGCUCCUACAUCU[dT][dT] |
| A | ID NO: 373) | S | (SEQ ID NO: 747) |
| OSL188 | UCGAUGUUACUGUUUUGCCGG[dT][dT] (SEQ | OSL188 | CCGGCAAAACAGUAACAUCGA[dT][dT] |
| A | ID NO: 374) | S | (SEQ ID NO: 748) |
| OSL189 | UUGCUAAAAAUGAGAUAGGGU[dT][dT] (SEQ | OSL189 | ACCCUAUCUCAUUUUUAGCAA[dT][dT] |
| A | ID NO: 375) | S | (SEQ ID NO: 749) |
| OSL190 | AUUUCUCAAAUAGUAACGGUU[dT][dT] (SEQ | OSL190 | AACCGUUACUAUUUGAGAAAU[dT][dT] |
| A | ID NO: 376) | S | (SEQ ID NO: 750) |
| OSL191 | AAUUUCUCAAAUAGUAACGGU[dT][dT] (SEQ | OSL191 | ACCGUUACUAUUUGAGAAAUU[dT][dT] |
| A | ID NO: 377) | S | (SEQ ID NO: 751) |
| OSL192 | AAAUUUCUCAAAUAGUAACGG[dT][dT] (SEQ | OSL192 | CCGUUACUAUUUGAGAAAUUU[dT][dT] |
| A | ID NO: 378) | S | (SEQ ID NO: 752) |
| OSL193 | AGUUAAAUUUCUCAAAUAGUA[dT][dT] (SEQ | OSL193 | UACUAUUUGAGAAAUUUAACU[dT][dT] |
| A | ID NO: 379) | S | (SEQ ID NO: 753) |
| OSL194 | AUCUAUAGUUAAAUUUCUCAA[dT][dT] (SEQ | OSL194 | UUGAGAAAUUUAACUAUAGAU[dT][dT] |
| A | ID NO: 380) | S | (SEQ ID NO: 754) |
| OSL195 | UAAAAAUAGCCAUCUAUAGUU[dT][dT] (SEQ | OSL195 | AACUAUAGAUGGCUAUUUUUA[dT][dT] |
| A | ID NO: 381) | S | (SEQ ID NO: 755) |
| OSL196 | AUCUAAUGCCACAACAUUGUC[dT][dT] (SEQ | OSL196 | GACAAUGUUGUGGCAUUAGAU[dT][dT] |
| A | ID NO: 382) | S | (SEQ ID NO: 756) |
| OSL197 | AAUCCAAUACAAUCUCUUCUC[dT][dT] (SEQ | OSL197 | GAGAAGAGAUUGUAUUGGAUU[dT][dT] |
| A | ID NO: 383) | S | (SEQ ID NO: 757) |
| OSL198 | ACAUUCUCUCAAUGACUUGCC[dT][dT] (SEQ | OSL198 | GGCAAGUCAUUGAGAGAAUGU[dT][dT] |
| A | ID NO: 384) | S | (SEQ ID NO: 758) |
| OSL199 | AUGAUUGUCUCCUUGUUUGUC[dT][dT] (SEQ | OSL199 | GACAAACAAGGAGACAAUCAU[dT][dT] |
| A | ID NO: 385) | S | (SEQ ID NO: 759) |
| OSL200 | AUUAUCACAGACUUGUUGGUU[dT][dT] (SEQ | OSL200 | AACCAACAAGUCUGUGAUAAU[dT][dT] |
| A | ID NO: 386) | S | (SEQ ID NO: 760) |
| OSL201 | UUCAAAAAUGGUAAUAGCGAA[dT][dT] (SEQ | OSL201 | UUCGCUAUUACCAUUUUUGAA[dT][dT] |
| A | ID NO: 387) | S | (SEQ ID NO: 761) |
| OSL202 | UCUUCAAAAAUGGUAAUAGCG[dT][dT] (SEQ | OSL202 | CGCUAUUACCAUUUUUGAAGA[dT][dT] |
| A | ID NO: 388) | S | (SEQ ID NO: 762) |
| OSL203 | AUUUGUUUCCCUUUUCCACUG[dT][dT] (SEQ | OSL203 | CAGUGGAAAAGGGAAACAAAU[dT][dT] |
| A | ID NO: 389) | S | (SEQ ID NO: 763) |
| OSL204 | AUUUGAUCCAUCAUAUUUGUU[dT][dT] (SEQ | OSL204 | AACAAAUAUGAUGGAUCAAAU[dT][dT] |
| A | ID NO: 390) | S | (SEQ ID NO: 764) |
| OSL205 | UAUAUGGAUGGUACACAUGGA[dT][dT] (SEQ | OSL205 | UCCAUGUGUACCAUCCAUAUA[dT][dT] |
| A | ID NO: 391) | S | (SEQ ID NO: 765) |
| OSL206 | AAAGAAGACGGUCUUCAUCAG[dT][dT] (SEQ | OSL206 | CUGAUGAAGACCGUCUUCUUU[dT][dT] |
| A | ID NO: 392) | S | (SEQ ID NO: 766) |
| OSL207 | UGAAUUCUGUGAAGUUGUCAC[dT][dT] (SEQ | OSL207 | GUGACAACUUCACAGAAUUCA[dT][dT] |
| A | ID NO: 393) | S | (SEQ ID NO: 767) |
| OSL208 | ACAAUGUCCACUUGUACACUG[dT][dT] (SEQ | OSL208 | CGGUGUACAAGUGGACAUUGU[dT][dT] |
| A | ID NO: 394) | S | (SEQ ID NO: 768) |
| OSL209 | ACACAAUGUCCACUUGUACAC[dT][dT] (SEQ | OSL209 | GUGUACAAGUGGACAUUGUGU[dT][dT] |
| A | ID NO: 395) | S | (SEQ ID NO: 769) |
| OSL210 | UUUUUGCAUUCGAACAUAGUA[dT][dT] (SEQ | OSL210 | UACUAUGUUCGAAUGCAAAAA[dT][dT] |
| A | ID NO: 396) | S | (SEQ ID NO: 770) |
| OSL211 | AUGGUUUUUGCAUUCGAACAU[dT][dT] (SEQ | OSL211 | AUGUUCGAAUGCAAAAACCAU[dT][dT] |
| A | ID NO: 397) | S | (SEQ ID NO: 771) |
| OSL212 | AUACAAACAUGGUUUUUGCAU[dT][dT] (SEQ | OSL212 | AUGCAAAAACCAUGUUUGUAU[dT][dT] |
| A | ID NO: 398) | S | (SEQ ID NO: 772) |
| OSL213 | AUCACAUUUCCAAUAUGGCGG[dT][dT] (SEQ | OSL213 | CCGCCAUAUUGGAAAUGUGAU[dT][dT] |
| A | ID NO: 399) | S | (SEQ ID NO: 773) |
| OSL214 | UGAAGUUCUUCAUCUGAACCA[dT][dT] (SEQ | OSL214 | UGGUUCAGAUGAAGAACUUCA[dT][dT] |
| A | ID NO: 400) | S | (SEQ ID NO: 774) |
| OSL215 | AUAAAUGCAGCGAUUGUUGUC[dT][dT] (SEQ | OSL215 | GACAACAAUCGCUGCAUUUAU[dT][dT] |
| A | ID NO: 401) | S | (SEQ ID NO: 775) |
| OSL216 | AUUCUGUACAAGGUUUAGGGG[dT][dT] (SEQ | OSL216 | CCCCUAAACCUUGUACAGAAU[dT][dT] |
| A | ID NO: 402) | S | (SEQ ID NO: 776) |
| OSL217 | UAUUCUGUACAAGGUUUAGGG[dT][dT] (SEQ | OSL217 | CCCUAAACCUUGUACAGAAUA[dT][dT] |
| A | ID NO: 403) | S | (SEQ ID NO: 777) |
| OSL218 | AUUCAUAUUCUGUACAAGGUU[dT][dT] (SEQ | OSL218 | AACCUUGUACAGAAUAUGAAU[dT][dT] |
| A | ID NO: 404) | S | (SEQ ID NO: 778) |
| OSL219 | UAUUCAUAUUCUGUACAAGGU[dT][dT] (SEQ | OSL219 | ACCUUGUACAGAAUAUGAAUA[dT][dT] |
| A | ID NO: 405) | S | (SEQ ID NO: 779) |
| OSL220 | UUAUAUUCAUAUUCUGUACAA[dT][dT] (SEQ | OSL220 | UUGUACAGAAUAUGAAUAUAA[dT][dT] |
| A | ID NO: 406) | S | (SEQ ID NO: 780) |
| OSL221 | UAUUGCAACCCAGUUCAUCGG[dT][dT] (SEQ | OSL221 | CCGAUGAACUGGGUUGCAAUA[dT][dT] |
| A | ID NO: 407) | S | (SEQ ID NO: 781) |
| OSL222 | AUAUUUUCAGCACAUGUUCUU[dT][dT] (SEQ | OSL222 | AAGAACAUGUGCUGAAAAUAU[dT][dT] |
| A | ID NO: 408) | S | (SEQ ID NO: 782) |
| OSL223 | UUAAUUGGGUACAAUUUUGCU[dT][dT] (SEQ | OSL223 | AGCAAAAUUGUACCCAAUUAA[dT][dT] |
| A | ID NO: 409) | S | (SEQ ID NO: 783) |
| OSL224 | AAAAACAUUGGUUUCGAACCC[dT][dT] (SEQ | OSL224 | GGGUUCGAAACCAAUGUUUUU[dT][dT] |
| A | ID NO: 410) | S | (SEQ ID NO: 784) |
| OSL225 | UGUCAAAAACAUUGGUUUCGA[dT][dT] (SEQ | OSL225 | UCGAAACCAAUGUUUUUGACA[dT][dT] |
| A | ID NO: 411) | S | (SEQ ID NO: 785) |
| OSL226 | UUCGAAUUCGGACAUUGUCAG[dT][dT] (SEQ | OSL226 | CUGACAAUGUCCGAAUUCGAA[dT][dT] |
| A | ID NO: 412) | S | (SEQ ID NO: 786) |
| OSL227 | AUUAUAUUUUCGAAUUCGGAC[dT][dT] (SEQ | OSL227 | GUCCGAAUUCGAAAAUAUAAU[dT][dT] |
| A | ID NO: 413) | S | (SEQ ID NO: 787) |
| OSL228 | AGAUUAUAUUUUCGAAUUCGG[dT][dT] (SEQ | OSL228 | CCGAAUUCGAAAAUAUAAUCU[dT][dT] |
| A | ID NO: 414) | S | (SEQ ID NO: 788) |
| OSL229 | UCAUCUUGAAGAUACUCUGAG[dT][dT] (SEQ | OSL229 | CUCAGAGUAUCUUCAAGAUGA[dT][dT] |
| A | ID NO: 415) | S | (SEQ ID NO: 789) |
| OSL230 | UAUAUUCCUCAUCUUGAAGAU[dT][dT] (SEQ | OSL230 | AUCUUCAAGAUGAGGAAUAUA[dT][dT] |
| A | ID NO: 416) | S | (SEQ ID NO: 790) |
| OSL231 | UUUGAUAGCACCAAACCUAGAGCCC[dT][dT] | OSL231 | GGGCUCUAGGUUUGGUGCUAUCAAA[dT] |
| A-1 | (SEQ ID NO: 417) | S-1 | [dT] (SEQ ID NO: 791) |
| OSL231 | UUUGAUAGCACCAAACCUAGAGCCC[dT]*[dT] | OSL231 | GGGCUCUAGGUUUGGUGCUAUCAAA[dT]* |
| A-2 | (SEQ ID NO: 418) | S-2 | [dT] (SEQ ID NO: 792) |
| OSL231 | [mU][mU][mU][mG][mA][mU][mA][mG][mC][ | OSL231 | [mG][mG][mG][mC][mU][mC][mU][mA][m |
| A-3 | mA][mC][mC][mA][mA][mA][mC][mC][mU][ | S-3 | G][mG][mU][mU][mU][mG][mG][mU][mG] |
| | mA][mG][mA][mG][mC][mC][mC][dT]*[dT] | | [mC][mU][mA][mU][mC][mA][mA][mA][dT] |
| | (SEQ ID NO: 419) | | *[dT] (SEQ ID NO: 793) |
| OSL231 | [mU][mU][mU][mG][mA][mU][mA][mG][mC][ | OSL231 | [mG][mG][mG][mC][mU][mC][mU][mA][m |
| A-4 | mA][mC][mC][mA][mA][mA][mC][mC][mU][ | S-4 | G][mG][mU][mU][mU][mG][mG][mU][mG] |
| | mA][mG][mA][mG][mC][mC][mC][dT]*[dT] | | [mC][mU][mA][mU][mC][mA][mA][mA] |
| | (SEQ ID NO: 420) | | (SEQ ID NO: 794) |
| OSL231 | [mU][mU][mU][mG][mA][mU][mA][mG][mC][ | OSL231 | [mG][mG][mG][mC][mU][mC][mU][mA][m |
| A-5 | mA][mC][mC][mA][mA][mA][mC][mC][mU][ | S-5 | G][mG][mU][mU][mU][mG][mG][mU][mG] |
| | mA][mG][mA][mG][mC][mC][mC] (SEQ ID | | [mC][mU][mA][mU][mC][mA][mA][mA][dT] |
| | NO: 421) | | *[dT] (SEQ ID NO: 795) |
| OSL231 | [mU][mU][mU][mG][mA][mU][mA][mG][mC][ | OSL231 | [mG][mG][mG][mC][mU][mC][mU][mA][m |
| A-6 | mA][mC][mC][mA][mA][mA][mC][mC][mU][ | S-6 | G][mG][mU][mU][mU][mG][mG][mU][mG] |
| | mA][mG][mA][mG][mC][mC][mC] (SEQ ID | | [mC][mU][mA][mU][mC][mA][mA][mA] |
| | NO: 422) | | (SEQ ID NO: 796) |
| OSL231 | [mU][mU][mU][mG][mA][mU][mA][mG][mC][ | OSL231 | GGGCUCUAGGUUUGGUGCUAUCAAA[dT]* |
| A-7 | mA][mC][mC][mA][mA][mA][mC][mC][mU][ | S-7 | [dT] (SEQ ID NO: 797) |
| | mA][mG][mA][mG][mC][mC][mC][dT]*[dT] | | |
| | (SEQ ID NO: 423) | | |
| OSL231 | [mU][2fU][mU][2fG][mA][2fU][mA][2fG] | OSL231 | [2fG][mG][2fG][mC][2fU][mC][2fU][mA] |
| A-8 | [mC][2fA][mC][2fC][mA][2fA][mA][2fC] | S-8 | [2fG][mG][2fU][mU][2fU][mG][2fG][mU] |
| | [mC][2fU][mA][2fG][mA][2fG][mC][2fC] | | [2fG][mC][2fU][mA][2fU][mC][2fA][mA] |
| | [mC][dT]*[dT] (SEQ ID NO: 424) | | [2fA][dT]*[dT] (SEQ ID NO: 798) |
| OSL231 | [mU][2fU][mU][2fG][mA][2fU][mA][2fG] | OSL231 | [2fG][mG][2fG][mC][2fU][mC][2fU][mA] |
| A-9 | [mC][2fA][mC][2fC][mA][2fA][mA][2fC] | S-9 | [2fG][mG][2fU][mU][2fU][mG][2fG][mU] |
| | [mC][2fU][mA][2fG][mA][2fG][mC][2fC] | | [2fG][mC][2fU][mA][2fU][mC][2fA][mA] |
| | [mC] (SEQ ID NO: 425) | | [2fA][dT]*[dT] (SEQ ID NO: 799) |
| OSL231 | [mU][2fU][mU][2fG][mA][2fU][mA][2fG] | OSL231 | [2fG][mG][2fG][mC][2fU][mC][2fU][mA] |
| A-10 | [mC][2fA][mC][2fC][mA][2fA][mA][2fC] | S-10 | [2fG][mG][2fU][mU][2fU][mG][2fG][mU] |
| | [mC][2fU][mA][2fG][mA][2fG][mC][2fC] | | [2fG][mC][2fU][mA][2fU][mC][2fA][mA] |
| | [mC][dT]*[dT] (SEQ ID NO: 426) | | [2fA] (SEQ ID NO: 800) |
| OSL231 | [mU][2fU][mU][2fG][mA][2fU][mA][2fG] | OSL231 | [2fG][mG][2fG][mC][2fU][mC][2fU][mA] |
| A-11 | [mC][2fA][mC][2fC][mA][2fA][mA][2fC] | S-11 | [2fG][mG][2fU][mU][2fU][mG][2fG][mU] |
| | [mC][2fU][mA][2fG][mA][2fC][mC][2fC] | | [2fG][mC][2fU][mA][2fU][mC][2fA][mA] |
| | [mC] (SEQ ID NO: 427) | | [2fA] (SEQ ID NO: 801) |
| OSL231 | [2fU][mU][2fU][mG][2fA][mU][2fA][mG] | OSL231 | [mG][2fG][mG][2fC][mU][2fC][mU][2fA] |
| A-12 | [2fC][mA][2fC][mC][2fA][mA][2fA][mC] | S-12 | [mG][2fG][mU][2fU][mU][2fG][mG][2Fu] |
| | [2fC][mU][2fA][mG][2fA][mG][2fC][mC] | | [mG][2fC][mU][2fA][mU][2fC][mA][2fA] |
| | [2fC][dT]*[dT] (SEQ ID NO: 428) | | [mA][dT]*[dT] (SEQ ID NO: 802) |
| OSL231 | [mU][2fA][mU][2fC][mA][2fA][mA][2fC] | OSL231 | [2fG][mG][2fG][mC][2fU][mC][2fU][mA] |
| A-13 | [mC][2fU][mC][2fG][mA][2fU][mA][2fG] | S-13 | [2fG][mG][2fU][mU][2fU][mG][2fG][mU] |
| | [mC][2fA][mA][2fC][mA][2fC][mC][2fG] | | [2fG][mC][2fU][mA][2fU][mC][2fA][mA] |
| | [mC] (SEQ I DNO: 429) | | [2fA]-LINKER-LIG (SEQ ID NO: 803) |
| OSL231 | [mU][2fU][2fU][2fG][2fA][2fU][2fA][2fG] | OSL231 | [2fU][mC][2fU][mA][2fG][mG][2fU][mU] |
| A-14 | [2fC][2fA][2fC][2fC][2fA][2fA][2fA] | S-14 | [2fU][mG][2fG][mU][2fG][mC][2fU][mA] |
| | [2fC][2fC][2fU][2fA][2fG][2fA][2fG] | | [2fU][mC][2fA][mA][2fA][dT]*[dT] |
| | [2fC][2fC][2fC][dT]*[dT] (SEQ ID | | (SEQ ID NO: 804) |
| | NO: 430) | | |
| OSL231 | [mU][2fU][2fU][2fG][2fA][2fU][2fA][2fG] | OSL231 | [2fU][mU][2fU][mG][2fG][mU][2fG][mC] |
| A-15 | [2fC][2fA][2fC][2fC][2fA][2fA][2fA] | S-15 | [2fU][mA][2fU][mC][2fA][mA][2fA][dT] |
| | [2fC][2fC][2fU][2fA][2fG][2fA][2fG] | | *[dT] (SEQ ID NO: 805) |
| | [2fC][2fC][2fC][dT]*[dT] (SEQ ID | | |
| | NO: 431) | | |
| OSL231 | [mU][2fU][mU][2fG][mA][2fU][mA][2fG] | OSL231 | [2fU][mU][2fU][mG][2fG][mU][2fG][mC] |
| A-16 | [mC][2fA][mC][2fC][mA][2fA][mA][2fC] | S-16 | [2fU][mA][2fU][mC][2fA][mA][2fA][dT] |
| | [mC][2fU][mA][2fG][mA][2fG][mC][2fC] | | *[dT] (SEQ ID NO: 806) |
| | [mC][dT]*[dT] (SEQ ID NO: 432) | | |
| OSL231 | [mU][2fU][mU][2fG][mA][2fU][mA][2fG] | OSL231 | [2fU][mU][2fU][mG][2fG][mU][2fG][mC] |
| A-17 | [mC][2fA][mC][2fC][mA][2fA][mA][2fC] | S-17 | [2fU][mA][2fU][mC][2fA][mA][2fA] |
| | [mC][2fU][mA][2fG][mA][2fG][mC] (SEQ | | (SEQ ID NO: 807) |
| | ID NO: 433) | | |
| OSL231 | [mU][2fU][mU][2fG][mA][2fU][mA][2fG] | OSL231 | [2fU][mU][2fU][mG][2fG][mU][2fG][mC] |
| A-18 | [mC][2fA][mC][2fC][mA][2fA][mA][2fC] | S-18 | [2fU][mA][2fU][mC][2fA][mA][2fA] |
| | [2fC][2fU][2fA][2fG][2fA][2fG][2fC] | | (SEQ ID NO: 808) |
| | (SEQ ID NO: 434) | | |
| OSL232 | UCAAAGUUGGGGAUGUAGGCA[dT][dT] (SEQ | OSL232 | UGCCUACAUCCCCAACUUUGA[dT][dT] |
| A | ID NO: 435) | S | (SEQ ID NO: 809) |
| OSL233 | UCAGUUUCAGGUCAACUUCCU[dT][dT] (SEQ | OSL233 | AGGAAGUUGACCUGAAACUGA[dT][dT] |
| A | ID NO: 436) | S | (SEQ ID NO: 810) |
| OSL234 | UACGUAUUUCAGUUUCAGGUC[dT][dT] (SEQ | OSL234 | GACCUGAAACUGAAAUACGUA[dT][dT] |
| A | ID NO: 437) | S | (SEQ ID NO: 811) |
| OSL235 | UUACGUAUUUCAGUUUCAGGU[dT][dT] (SEQ | OSL235 | ACCUGAAACUGAAAUACGUAA[dT][dT] |
| A | ID NO: 438) | S | (SEQ ID NO: 812) |
| OSL236 | AGUUUAGCCACCUCAAUGCGU[dT][dT] (SEQ | OSL236 | ACGCAUUGAGGUGGCUAAACU[dT][dT] |
| A | ID NO: 439) | S | (SEQ ID NO: 813) |
| OSL237 | AACAUAAGCCCUAGUUUGGGA[dT][dT] (SEQ | OSL237 | UCCCAAACUAGGGCUUAUGUU[dT][dT] |
| A | ID NO: 440) | S | (SEQ ID NO: 814) |
| OSL238 | UCGAUUUUAGGUUCCUUUCCC[dT][dT] (SEQ | OSL238 | GGGAAAGGAACCUAAAAUCGA[dT][dT] |
| A | ID NO: 441) | S | (SEQ ID NO: 815) |
| OSL239 | UCGAAAACCAGGAUGUUGCGG[dT][dT] (SEQ | OSL239 | CCGCAACAUCCUGGUUUUCGA[dT][dT] |
| A | ID NO: 442) | S | (SEQ ID NO: 816) |
| OSL240 | UCAAAUAAUCGAUAGAAAGGC[dT][dT] (SEQ | OSL240 | GCCUUUCUAUCGAUUAUUUGA[dT][dT] |
| A | ID NO: 443) | S | (SEQ ID NO: 817) |
| OSL241 | UUGUUCAAAUAAUCGAUAGAA[dT][dT] (SEQ | OSL241 | UUCUAUCGAUUAUUUGAACAA[dT][dT] |
| A | ID NO: 444) | S | (SEQ ID NO: 818) |
| OSL242 | UUAUGGUUUCAAUAACGUCCU[dT][dT] (SEQ | OSL242 | AGGACGUUAUUGAAACCAUAA[dT][dT] |
| A | ID NO: 445) | S | (SEQ ID NO: 819) |
| OSL243 | UUUUAUGGUUUCAAUAACGUC[dT][dT] (SEQ | OSL243 | GACGUUAUUGAAACCAUAAAA[dT][dT] |
| A | ID NO: 446) | S | (SEQ ID NO: 820) |
| OSL244 | AUUUUAUGGUUUCAAUAACGU[dT][dT] (SEQ | OSL244 | ACGUUAUUGAAACCAUAAAAU[dT][dT] |
| A | ID NO: 447) | S | (SEQ ID NO: 821) |
| OSL245 | UUUGCAAUGACUCUCCUAUCAGUCC[dT][dT] | OSL245 | GGACUGAUAGGAGAGUCAUUGCAAA[dT][dT] |
| A-1 | (SEQ ID NO: 448) | S-1 | (SEQ ID NO: 822) |
| OSL245 | UUUGCAAUGACUCUCCUAUCAGUCC[dT]*[dT] | OSL245 | GGACUGAUAGGAGAGUCAUUGCAA]A[dT] |
| A-2 | (SEQ ID NO: 449) | S-2 | *[dT] (SEQ ID NO: 823) |
| OSL245 | [mU][mU][mU][mG][mC][mA][mA][mU][mG][ | OSL245 | [mG][mG][mA][mC][mU][mG][mA][mU][m |
| A-3 | mA][mC][mU][mC][mU][mC][mC][mU][mA][ | S-3 | A][mG][mG][mA][mG][mA][mG][mU][mC][ |
| | mU][mC][mA][mG][mU][mC][mC][dT]*[dT] | | mA][mU][mU][mG][mC][mA][mA][mA][dT |
| | (SEQ ID NO: 450) | | ]*[dT] (SEQ ID NO: 824) |
| OSL245 | [mU][mU][mU][mG][mC][mA][mA][mU][mG][ | OSL245 | [mG][mG][mA][mC][mU][mG][mA][mU][m |
| A-4 | mA][mC][mU][mC][mU][mC][mC][mU][mA][ | S-4 | A][mG][mG][mA][mG][mA][mG][mU][mC][ |
| | mU][mC][mA][mG][mU][mC][mC][dT]*[dT] | | mA][mU][mU][mG][mC][mA][mA][mA](SE |
| | (SEQ ID NO: 451) | | Q ID NO: 825) |
| OSL245 | [mU][mU][mU][mG][mC][mA][mA][mU][mG][ | OSL245 | [mG][mG][mA][mC][mU][mG][mA][mU][m |
| A-5 | mA][mC][mU][mC][mU][mC][mC][mU][mA][ | S-5 | A][mG][mG][mA][mG][mA][mG][mU][mC][ |
| | mU][mC][mA][mG][mU][mC][mC] (SEQ ID | | mA][mU][mU][mG][mC][mA][mA][mA][dT] |
| | NO: 452) | | *[dT](SEQ ID NO: 826) |
| OSL245 | [mU][mU][mU][mG][mC][mA][mA][mU][mG][ | OSL245 | [mG][mG][mA][mC][mU][mG][mA][mU][m |
| A-6 | mA][mC][mU][mC][mU][mC][mC][mU][mA][ | S-6 | A][mG][mG][mA][mG][mA][mG][mU][mC][ |
| | mU][mC][mA][mG][mU][mC][mC] (SEQ ID | | mA][mU][mU][mG][mC][mA][mA][mA] |
| | NO: 453) | | (SEQ ID NO: 827) |
| OSL245 | [mU][mU][mU][mG][mC][mA][mA][mU][mG][ | OSL245 | GGACUGAUAGGAGAGUCAUUGCAAA[dT]* |
| A-7 | mA][mC][mU][mC][mU][mC][mC][mU][mA][ | S-7 | [dT] (SEQ ID NO: 828) |
| | mU][mC][mA][mG][mU][mC][mC][dT]*[dT] | | |
| | (SEQ ID NO: 454) | | |
| OSL245 | [mU][2fU][mU][2fG][mC][2fA][mA][2fU] | OSL245 | [2fG][mG][2fA][mC][2fU][mG][2fA][mU] |
| A-8 | [mG][2fA][mC][2fU][mC][2fU][mC][2fC] | S-8 | [2fA][mG][2fG][mA][2fG][mA][2fG][mU] |
| | [mU][2fA][mU][2fC][mA][2fG][mU][2fC] | | [2fC][mA][2fU][mU][2fG][mC][2fA][mA] |
| | [mC][dT]*[dT] (SEQ ID NO: 455) | | [2fA][dT]*[dT] (SEQ ID NO: 829) |
| OSL245 | [mU][2fU][mU][2fG][mC][2fA][mA][2fU] | OSL245 | [2fG][mG][2fA][mC][2fU][mG][2fA][mU] |
| A-9 | [mG][2fA][mC][2fU][mC][2fU][mC][2fC] | S-9 | [2fA][mG][2fG][mA][2fG][mA][2fG][mU] |
| | [mU][2fA][mU][2fC][mA][2fG][mU][2fC] | | [2fC][mA][2fU][mU][2fG][mC][2fA][mA] |
| | [mC] (SEQ ID NO: 456) | | [2fA][dT]*[dT] (SEQ ID NO: 830) |
| OSL245 | [mU][2fU][mU][2fG][mC][2fA][mA][2fU] | OSL245 | [2fG][mG][2fA][mC][2fU][mG][2fA][mU] |
| A-10 | [mG][2fA][mC][2fU][mC][2fU][mC][2fC] | S-10 | [2fA][mG][2fG][mA][2fG][mA][2fG][mU] |
| | [mU][2fA][mU][2fC][mA][2fG][mU][2fC] | | [2fC][mA][2fU][mU][2fG][mC][2fA][mA] |
| | [mC][dT]*[dT ](SEQ ID NO: 457) | | [2fA] (SEQ ID NO: 831) |
| OSL245 | [mU][2fU][mU][2fG][mC][2fA][mA][2fU] | OSL245 | [2fG][mG][2fA][mC][2fU][mG][2fA][mU] |
| A-11 | [mG][2fA][mC][2fU][mC][2fU][mC][2fC] | S-11 | [2fA][mG][2fG][mA][2fG][mA][2fG][mU] |
| | [mU][2fA][mU][2fC][mA][2fC][mU][2fC] | | [2fC][mA][2fU][mU][2fG][mC][2fA][mA] |
| | [mC] (SEQ ID NO: 458) | | [2fA] (SEQ ID NO: 832) |
| OSL245 | [2fU][mU][2fU][mG][2fC][mA][2fA][mU] | OSL245 | [mG][2fG][mA][2fC][mU][2fG][mA][2fU] |
| A-12 | [2fG][mA][2fC][mU][2fC][mU][2fC][mC] | S-12 | [mA][2fG][mG][2fA][mG][2fA][mG][2fU] |
| | [2fU][mA][2fU][mC][2fA][mG][2fU][mC] | | [mC][2fA][mU][2fU][mG][2fC][mA][2fA] |
| | [2fC][dT]*[dT] (SEQ ID NO: 459) | | [mA][dT]*[dT] (SEQ ID NO: 833) |
| OSL245 | [mU][2fA][mU][2fC][mC][2fU][mA][2fA] | OSL245 | [2fG][mG][2fA][mC][2fU][mG][2fA][mU] |
| A-13 | [mG][2fU][mC][2fA][mC][2fA][mC][2fG] | S-13 | [2fA][mG][2fG][mA][2fG][mA][2fG][mU] |
| | [mU][2fU][mU][2fG][mA][2fC][mU][2fG] | | [2fC][mA][2fU][mU][2fG][mC][2fA][mA] |
| | [mC] (SEQ ID NO: 460) | | [2fA] (SEQ ID NO: 834) |
| OSL245 | [mU][2fU][2fU][2fG][2fC][2fA][2fA][2fU] | OSL245 | [2fU][mG][2fA][mU][2fA][mG][2fG][mA] |
| A-14 | [2fG][2fA][2fC][2fU][2fC][2fU][2fC] | S-14 | [2fG][mA][2fG][mU][2fC][mA][2fU][mU] |
| | [2fC][2fU][2fA][2fU][2fC][2fA][2fG] | | [2fG][mC][2fA][mA][2fA][dT]*[dT] |
| | [2fU][2fC][2fC][dT]*[dT] (SEQ ID NO: | | (SEQ ID NO: 835) |
| | 461) | | |
| OSL245 | [mU][2fU][2fU][2fG][2fC][2fA][2fA][2fU] | OSL245 | [2fG][mA][2fG][mA][2fG][mU][2fC][mA] |
| A-15 | [2fG][2fA][2fC][2fU][2fC][2fU][2fC] | S-15 | [2fU][mU][2fG][mC][2fA][mA][2fA][dT] |
| | [2fC][2fU][2fA][2fU][2fC][2fA][2fG] | | *[dT] (SEQ ID NO: 836) |
| | [2fU][2fC][2fC][dT]*[dT] (SEQ ID | | |
| | NO: 462) | | |
| OSL245 | [mU][2fU][mU][2fG][mC][2fA][mA][2fU] | OSL245 | [2fG][mA][2fG][mA][2fG][mU][2fC][mA] |
| A-16 | [mG][2fA][mC][2fU][mC][2fU][mC][2fC] | S-16 | [2fU][mU][2fG][mC][2fA][mA][2fA][dT] |
| | [mU][2fA][mU][2fC][mA][2fG][mU][2fC] | | *[dT] (SEQ ID NO: 837) |
| | [mC][dT]*[dT] (SEQ ID NO: 463) | | |
| OSL245 | [mU][2fU][mU][2fG][mC][2fA][mA][2fU] | OSL245 | [2fG][mA][2fG][mA][2fG][mU][2fC][mA] |
| A-17 | [mG][2fA][mC][2fU][mC][2fU][mC][2fC] | S-17 | [2fU][mU][2fG][mC][2fA][mA][2fA] |
| | [mU][2fA][mU][2fC][mA][2fG][mU] (SEQ | | (SEQ ID NO: 838) |
| | ID NO: 464) | | |
| OSL245 | [mU][2fU][mU][2fG][mC][2fA][mA][2fU] | OSL245 | ACCAGUUAUACUGGAUAUCUA[dT][dT] |
| A-18 | [mG][2fA][mC][2fU][mC][2fU][mC][2fC] | S-18 | (SEQ ID NO: 840) |
| | [2fU][2fA][2fU][2fC][2fA][2fG][2fU] | | |
| | (SEQ ID NO: 465) | | |
| OSL246 | UAGAUAUCCAGUAUAACUGGU[dT][dT] (SEQ | OSL246 | GGGAGAAGUAUGGAAACAAAA[dT][dT] |
| A | ID NO: 466) | S | (SEQ ID NO: 841) |
| OSL247 | UUUUGUUUCCAUACUUCUCCC[dT][dT] (SEQ | OSL247 | AAGUAUGGAAACAAAAUAAAU[dT][dT] |
| A | ID NO: 467) | S | (SEQ ID NO: 842) |
| OSL248 | AUUUAUUUUGUUUCCAUACUU[dT][dT] (SEQ | OSL248 | UUCAUCAACUCAGAUACAAUA[dT][dT] |
| A | ID NO: 468) | S | (SEQ ID NO: 843) |
| OSL249 | UAUUGUAUCUGAGUUGAUGAA[dT][dT] (SEQ | OSL249 | CGGAGGAAAUUGCUAUUUUGA[dT][dT] |
| A | ID NO: 469) | S | (SEQ ID NO: 844) |
| OSL250 | UCAAAAUAGCAAUUUCCUCCG[dT][dT] (SEQ | OSL250 | AGGAAAUUGCUAUUUUGAUGA[dT][dT] |
| A | ID NO: 470) | S | (SEQ ID NO: 845) |
| OSL251 | UCAUCAAAAUAGCAAUUUCCU[dT][dT] (SEQ | OSL251 | CACCGGAAAAUAUUGUGAAAU[dT][dT] |
| A | ID NO: 471) | S | (SEQ ID NO: 846) |
| OSL252 | AUUUCACAAUAUUUUCCGGUG[dT][dT] (SEQ | OSL252 | UUGUGAAAUGGCGUUUUCAAA[dT][dT] |
| A | ID NO: 472) | S | (SEQ ID NO: 847) |
| OSL253 | UUUGAAAACGCCAUUUCACAA[dT][dT] (SEQ | OSL253 | CAGGAUUCUUCCACUAUAGAA[dT][dT] |
| A | ID NO: 473) | S | (SEQ ID NO: 848) |
| OSL254 | UUCUAUAGUGGAAGAAUCCUG[dT][dT] (SEQ | OSL254 | GGCAGAUCUUAACAUGGAUAU[dT][dT] |
| A | ID NO: 474) | S | (SEQ ID NO: 849) |
| OSL255 | AUAUCCAUGUUAAGAUCUGCC[dT][dT] (SEQ | OSL255 | GGCAAUGAGUGAAGACUUUGU[dT][dT] |
| A | ID NO: 475) | S | (SEQ ID NO: 850) |
| OSL256 | ACAAAGUCUUCACUCAUUGCC[dT][dT] (SEQ | OSL256 | AUCUGAAAAUGUGGAUAAUAA[dT][dT] |
| A | ID NO: 476) | S | (SEQ ID NO: 851) |
| OSL257 | UUAUUAUCCACAUUUUCAGAU[dT][dT] (SEQ | OSL257 | ACCAGUUAUACUGGAUAUCUA[dT][dT] |
| A | ID NO: 477) | S | (SEQ ID NO: 840) |
| OSL258 | UCUUAUUAUCCACAUUUUCAG[dT][dT] (SEQ | OSL258 | CUGAAAAUGUGGAUAAUAAGA[dT][dT] |
| A | ID NO: 478) | S | (SEQ ID NO: 852) |
| OSL259 | UCCAUAAUUCUUAUUAUCCAC[dT][dT] (SEQ | OSL259 | GUGGAUAAUAAGAAUUAUGGA[dT][dT] |
| A | ID NO: 479) | S | (SEQ ID NO: 853) |
| OSL260 | UUCCAUAAUUCUUAUUAUCCA[dT][dT] (SEQ | OSL260 | UGGAUAAUAAGAAUUAUGGAA[dT][dT] |
| A | ID NO: 480) | S | (SEQ ID NO: 854) |
| OSL261 | UUUUCGUUUGAAGAGAUUCCA[dT][dT] (SEQ | OSL261 | UGGAAUCUCUUCAAACGAAAA[dT][dT] |
| A | ID NO: 481) | S | (SEQ ID NO: 855) |
| OSL262 | UAGAUUUUCGUUUGAAGAGAU[dT][dT] (SEQ | OSL262 | AUCUCUUCAAACGAAAAUCUA[dT][dT] |
| A | ID NO: 482) | S | (SEQ ID NO: 856) |
| OSL263 | UUUAGAUUUUCGUUUGAAGAG[dT][dT] (SEQ | OSL263 | CUCUUCAAACGAAAAUCUAAA[dT][dT] |
| A | ID NO: 483) | S | (SEQ ID NO: 857) |
| OSL264 | UUGUUUAGAUUUUCGUUUGAA[dT][dT] (SEQ | OSL264 | UUCAAACGAAAAUCUAAACAA[dT][dT] |
| A | ID NO: 484) | S | (SEQ ID NO: 858) |
| OSL265 | UAGUUUGUUUAGAUUUUCGUU[dT][dT] | OSL265 | AACGAAAAUCUAAACAAACUA[dT][dT] |
| A | (SEQ ID NO: 485) | S | (SEQ ID NO: 859) |
| OSL266 | UUUCAAAGUUGGUAGUUUGUU[dT][dT] (SEQ | OSL266 | AACAAACUACCAACUUUGAAA[dT][dT] |
| A | ID NO: 486) | S | (SEQ ID NO: 860) |
| OSL267 | AUUGGAUUUUCAAAGUUGGUA[dT][dT] (SEQ | OSL267 | UACCAACUUUGAAAAUCCAAU[dT][dT] |
| A | ID NO: 487) | S | (SEQ ID NO: 861) |
| OSL268 | AUAGAUUGGAUUUUCAAAGUU[dT][dT] (SEQ | OSL268 | AACUUUGAAAAUCCAAUCUAU[dT][dT] |
| A | ID NO: 488) | S | (SEQ ID NO: 862) |
| OSL269 | UUAAAAGUGUCUUCUGUUGCA[dT][dT] (SEQ | OSL269 | UGCAACAGAAGACACUUUUAA[dT][dT] |
| A | ID NO: 489) | S | (SEQ ID NO: 863) |
| OSL270 | UCUUUAACAAGAUUUGCGGUG[dT][dT] (SEQ | OSL270 | CACCGCAAAUCUUGUUAAAGA[dT][dT] |
| A | ID NO: 490) | S | (SEQ ID NO: 864) |
| OSL271 | UCUUCUUUAACAAGAUUUGCG[dT][dT] (SEQ | OSL271 | CGCAAAUCUUGUUAAAGAAGA[dT][dT] |
| A | ID NO: 491) | S | (SEQ ID NO: 865) |
| OSL272 | UGGUAUAGCUAUACUUCAGAG[dT][dT] (SEQ | OSL272 | CUCUGAAGUAUAGCUAUACCA[dT][dT] |
| A | ID NO: 492) | S | (SEQ ID NO: 866) |
| OSL273 | AUUAUUCCCUAAAUAGCUGGU[dT][dT] (SEQ | OSL273 | ACCAGCUAUUUAGGGAAUAAU[dT][dT] |
| A | ID NO: 493) | S | (SEQ ID NO: 867) |
| OSL274 | UAAUUAUUCCCUAAAUAGCUG[dT][dT] (SEQ | OSL274 | CAGCUAUUUAGGGAAUAAUUA[dT][dT] |
| A | ID NO: 494) | S | (SEQ ID NO: 868) |
| OSL275 | AUAUAUGUGCAAAAGUGUGUU[dT][dT] (SEQ | OSL275 | AACACACUUUUGCACAUAUAU[dT][dT] |
| A | ID NO: 495) | S | (SEQ ID NO: 869) |
| OSL276 | AAAUAUAUGUGCAAAAGUGUG[dT][dT] (SEQ | OSL276 | CACACUUUUGCACAUAUAUUU[dT][dT] |
| A | ID NO: 496) | S | (SEQ ID NO: 870) |
| OSL277 | AACUUUUUUCAUCUGUUUGUA[dT][dT] (SEQ | OSL277 | UACAAACAGAUGAAAAAAGUU[dT][dT] |
| A | ID NO: 497) | S | (SEQ ID NO: 871) |
| OSL278 | UAAAGUACUGAAUGUUAACUU[dT][dT] (SEQ | OSL278 | AAGUUAACAUUCAGUACUUUA[dT][dT] |
| A | ID NO: 498) | S | (SEQ ID NO: 872) |
| OSL279 | UUUUUUUCAUAAAGUACUGAA[dT][dT] (SEQ | OSL279 | UUCAGUACUUUAUGAAAAAAA[dT][dT] |
| A | ID NO: 499) | S | (SEQ ID NO: 873) |
| OSL280 | UAUUUUUUUCAUAAAGUACUG[dT][dT] (SEQ | OSL280 | CAGUACUUUAUGAAAAAAAUA[dT][dT] |
| A | ID NO: 500) | S | (SEQ ID NO: 874) |
| OSL281 | AUUUGUAAAAAUAUGAGACGG[dT][dT] (SEQ | OSL281 | CCGUCUCAUAUUUUUACAAAU[dT][dT] |
| A | ID NO: 501) | S | (SEQ ID NO: 875) |
| OSL282 | ACAUUGUGAUAAUUAUUUGUA[dT][dT] (SEQ | OSL282 | UACAAAUAAUUAUCACAAUGU[dT][dT] |
| A | ID NO: 502) | S | (SEQ ID NO: 876) |
| OSL283 | AUACAUAUAGUACAUUGUGAU[dT][dT] (SEQ | OSL283 | AUCACAAUGUACUAUAUGUAU[dT][dT] |
| A | ID NO: 503) | S | (SEQ ID NO: 877) |
| OSL284 | AUAUACAUAUAGUACAUUGUG[dT][dT] (SEQ | OSL284 | CACAAUGUACUAUAUGUAUAU[dT][dT] |
| A | ID NO: 504) | S | (SEQ ID NO: 878) |
| OSL285 | AAAGAUAUACAUAUAGUACAU[dT][dT] (SEQ | OSL285 | AUGUACUAUAUGUAUAUCUUU[dT][dT] |
| A | ID NO: 505) | S | (SEQ ID NO: 879) |
| OSL286 | AUUACCUUCAGACAACUUCAG[dT][dT] (SEQ | OSL286 | CUGAAGUUGUCUGAAGGUAAU[dT][dT] |
| A | ID NO: 506) | S | (SEQ ID NO: 880) |
| OSL287 | UAUUUAUAGUAUUACCUUCAG[dT][dT] (SEQ | OSL287 | CUGAAGGUAAUACUAUAAAUA[dT][dT] |
| A | ID NO: 507) | S | (SEQ ID NO: 881) |
| OSL288 | UAAUCUUUCCAAAAUUUACAA[dT][dT] (SEQ | OSL288 | UUGUAAAUUUUGGAAAGAUUA[dT][dT] |
| A | ID NO: 508) | S | (SEQ ID NO: 882) |
| OSL289 | AGUAACAGGAUAAUCUUUCCA[dT][dT] (SEQ | OSL289 | UGGAAAGAUUAUCCUGUUACU[dT][dT] |
| A | ID NO: 509) | S | (SEQ ID NO: 883) |
| OSL290 | AUUCAGUAACAGGAUAAUCUU[dT][dT] (SEQ | OSL290 | AAGAUUAUCCUGUUACUGAAU[dT][dT] |
| A | ID NO: 510) | S | (SEQ ID NO: 884) |
| OSL291 | UAGCAAAUUCAGUAACAGGAU[dT][dT] (SEQ | OSL291 | AUCCUGUUACUGAAUUUGCUA[dT][dT] |
| A | ID NO: 511) | S | (SEQ ID NO: 885) |
| OSL292 | UUAGCAAAUUCAGUAACAGGA[dT][dT] (SEQ | OSL292 | UCCUGUUACUGAAUUUGCUAA[dT][dT] |
| A | ID NO: 512) | S | (SEQ ID NO: 886) |
| OSL293 | UCUUUAUUAGCAAAUUCAGUA[dT][dT] (SEQ | OSL293 | UACUGAAUUUGCUAAUAAAGA[dT][dT] |
| A | ID NO: 513) | S | (SEQ ID NO: 887) |
| OSL294 | AUCAUUUACUAUAAUGAUCAC[dT][dT] (SEQ | OSL294 | GUGAUCAUUAUAGUAAAUGAU[dT][dT] |
| A | ID NO: 514) | S | (SEQ ID NO: 888) |
| OSL295 | UUCUUGUUGGAUCAUUUACUA[dT][dT] (SEQ | OSL295 | UAGUAAAUGAUCCAACAAGAA[dT][dT] |
| A | ID NO: 515) | S | (SEQ ID NO: 889) |
| OSL296 | UCAAUUCCUUUUCUUGUUGGA[dT][dT] (SEQ | OSL296 | UCCAACAAGAAAAGGAAUUGA[dT][dT] |
| A | ID NO: 516) | S | (SEQ ID NO: 890) |
| OSL297 | AUUUUAUAGGAAAUAUGAGUG[dT][dT] (SEQ | OSL297 | CACUCAUAUUUCCUAUAAAAU[dT][dT] |
| A | ID NO: 517) | S | (SEQ ID NO: 891) |
| OSL298 | UAAUUUUAUAGGAAAUAUGAG[dT][dT] (SEQ | OSL298 | CUCAUAUUUCCUAUAAAAUUA[dT][dT] |
| A | ID NO: 518) | S | (SEQ ID NO: 892) |
| OSL299 | UGCUAAUGUGUAAAAAUGGAC[dT][dT] (SEQ | OSL299 | GUCCAUUUUUACACAUUAGCA[dT][dT] |
| A | ID NO: 519) | S | (SEQ ID NO: 893) |
| OSL300 | UUGAACAUUAAUUAAGUGCUA[dT][dT] (SEQ | OSL300 | UAGCACUUAAUUAAUGUUCAA[dT][dT] |
| A | ID NO: 520) | S | (SEQ ID NO: 894) |
| OSL301 | AUUGAACAUUAAUUAAGUGCU[dT][dT] (SEQ | OSL301 | AGCACUUAAUUAAUGUUCAAU[dT][dT] |
| A | ID NO: 521) | S | (SEQ ID NO: 895) |
| OSL302 | AUAUUGAACAUUAAUUAAGUG[dT][dT] (SEQ | OSL302 | CACUUAAUUAAUGUUCAAUAU[dT][dT] |
| A | ID NO: 522) | S | (SEQ ID NO: 896) |
| OSL303 | AAAUUGACAUGUAAUAUUGAA[dT][dT] (SEQ | OSL303 | UUCAAUAUUACAUGUCAAUUU[dT][dT] |
| A | ID NO: 523) | S | (SEQ ID NO: 897) |
| OSL304 | AUCAACAUAGCCAUUAAUCAA[dT][dT] (SEQ | OSL304 | UUGAUUAAUGGCUAUGUUGAU[dT][dT] |
| A | ID NO: 524) | S | (SEQ ID NO: 898) |
| OSL305 | UCUAUACAACACAUAGUGGCC[dT][dT] (SEQ | OSL305 | GGCCACUAUGUGUUGUAUAGA[dT][dT] |
| A | ID NO: 525) | S | (SEQ ID NO: 899) |
| OSL306 | AUGUCUAUACAACACAUAGUG[dT][dT] (SEQ | OSL306 | CACUAUGUGUUGUAUAGACAU[dT][dT] |
| A | ID NO: 526) | S | (SEQ ID NO: 900) |
| OSL307 | ACUGAAUUGCUUUUCCUACCU[dT][dT] (SEQ | OSL307 | AGGUAGGAAAAGCAAUUCAGU[dT][dT] |
| A | ID NO: 527) | S | (SEQ ID NO: 901) |
| OSL308 | AAAUAAAAAUGUUGUCUUGGC[dT][dT] (SEQ | OSL308 | GCCAAGACAACAUUUUUAUUU[dT][dT] |
| A | ID NO: 528) | S | (SEQ ID NO: 902) |
| OSL309 | AUCACAAAUAAAAAUGUUGUC[dT][dT] (SEQ | OSL309 | GACAACAUUUUUAUUUGUGAU[dT][dT] |
| A | ID NO: 529) | S | (SEQ ID NO: 903) |
| OSL310 | AAUGAUAUGGGAUUUCCUCAU[dT][dT] (SEQ | OSL310 | AUGAGGAAAUCCCAUAUCAUU[dT][dT] |
| A | ID NO: 530) | S | (SEQ ID NO: 904) |
| OSL311 | AUUAACCACAAACUCAAUGCA[dT][dT] (SEQ | OSL311 | UGCAUUGAGUUUGUGGUUAAU[dT][dT] |
| A | ID NO: 531) | S | (SEQ ID NO: 905) |
| OSL312 | UUUAAUUAACCACAAACUCAA[dT][dT] (SEQ | OSL312 | UUGAGUUUGUGGUUAAUUAAA[dT][dT] |
| A | ID NO: 532) | S | (SEQ ID NO: 906) |
| OSL313 | UUUGGUUUCAGAAAUUCAGCU[dT][dT] (SEQ | OSL313 | AGCUGAAUUUCUGAAACCAAA[dT][dT] |
| A | ID NO: 533) | S | (SEQ ID NO: 907) |
| OSL314 | UUAUGAAGACACAGAUUUGGU[dT][dT] (SEQ | OSL314 | ACCAAAUCUGUGUCUUCAUAA[dT][dT] |
| A | ID NO: 534) | S | (SEQ ID NO: 908) |
| OSL315 | UUUCAUAGAAACAAAAACCCA[dT][dT] (SEQ | OSL315 | UGGGUUUUUGUUUCUAUGAAA[dT][dT] |
| A | ID NO: 535) | S | (SEQ ID NO: 909) |
| OSL316 | AUGAUAUUUUCAUAGAAACAA[dT][dT] (SEQ | OSL316 | UUGUUUCUAUGAAAAUAUCAU[dT][dT] |
| A | ID NO: 536) | S | (SEQ ID NO: 910) |
| OSL317 | UAUAAUGAUAUUUUCAUAGAA[dT][dT] (SEQ | OSL317 | UUCUAUGAAAAUAUCAUUAUA[dT][dT] |
| A | ID NO: 537) | S | (SEQ ID NO: 911) |
| OSL318 | UGAUUAUAAUGAUAUUUUCAU[dT][dT] (SEQ | OSL318 | AUGAAAAUAUCAUUAUAAUCA[dT][dT] |
| A | ID NO: 538) | S | (SEQ ID NO: 912) |
| OSL319 | AUAAAUAGUGAUUAUAAUGAU[dT][dT] (SEQ | OSL319 | AUCAUUAUAAUCACUAUUUAU[dT][dT] |
| A | ID NO: 539) | S | (SEQ ID NO: 913) |
| OSL320 | AAAAGCUUAAUAAGAAUGGUU[dT][dT] (SEQ | OSL320 | AACCAUUCUUAUUAAGCUUUU[dT][dT] |
| A | ID NO: 540) | S | (SEQ ID NO: 914) |
| OSL321 | AAAAAGCUUAAUAAGAAUGGU[dT][dT] (SEQ | OSL321 | ACCAUUCUUAUUAAGCUUUUU[dT][dT] |
| A | ID NO: 541) | S | (SEQ ID NO: 915) |
| OSL322 | UAAAUGUACACAUUUAGCCAC[dT][dT] (SEQ | OSL322 | GUGGCUAAAUGUGUACAUUUA[dT][dT] |
| A | ID NO: 542) | S | (SEQ ID NO: 916) |
| OSL323 | AUAAAUGUACACAUUUAGCCA[dT][dT] (SEQ | OSL323 | UGGCUAAAUGUGUACAUUUAU[dT][dT] |
| A | ID NO: 543) | S | (SEQ ID NO: 917) |
| OSL324 | UAUAAAUGUACACAUUUAGCC[dT][dT] (SEQ | OSL324 | GGCUAAAUGUGUACAUUUAUA[dT][dT] |
| A | ID NO: 544) | S | (SEQ ID NO: 918) |
| OSL325 | UUCUAAUAUAAAUGUACACAU[dT][dT] (SEQ | OSL325 | AUGUGUACAUUUAUAUUAGAA[dT][dT] |
| A | ID NO: 545) | S | (SEQ ID NO: 919) |
| OSL326 | AAGAAUUAAAGAAAAGAUCUG[dT][dT] (SEQ | OSL326 | CAGAUCUUUUCUUUAAUUCUU[dT][dT] |
| A | ID NO: 546) | S | (SEQ ID NO: 920) |
| OSL327 | AAUAAGAAUUAAAGAAAAGAU[dT][dT] (SEQ | OSL327 | AUCUUUUCUUUAAUUCUUAUU[dT][dT] |
| A | ID NO: 547) | S | (SEQ ID NO: 921) |
| OSL328 | AAACCAAUAAGAAUUAAAGAA[dT][dT] (SEQ | OSL328 | UUCUUUAAUUCUUAUUGGUUU[dT][dT] |
| A | ID NO: 548) | S | (SEQ ID NO: 922) |
| OSL329 | ACUAUACCCACUAUUUAAGAG[dT][dT] (SEQ | OSL329 | CUCUUAAAUAGUGGGUAUAGU[dT][dT] |
| A | ID NO: 549) | S | (SEQ ID NO: 923) |
| OSL330 | ACAAAUGUGCAAUAUUAGCAC[dT][dT] (SEQ | OSL330 | GUGCUAAUAUUGCACAUUUGU[dT][dT] |
| A | ID NO: 550) | S | (SEQ ID NO: 924) |
| OSL331 | AACAAAUGUGCAAUAUUAGCA[dT][dT] (SEQ | OSL331 | UGCUAAUAUUGCACAUUUGUU[dT][dT] |
| A | ID NO: 551) | S | (SEQ ID NO: 925) |
| OSL332 | AUGUUUCAUUCAUUCAUCCAU[dT][dT] (SEQ | OSL332 | AUGGAUGAAUGAAUGAAACAU[dT][dT] |
| A | ID NO: 552) | S | (SEQ ID NO: 926) |
| OSL333 | AGUAGUAUAUGUUUCAUUCAU[dT][dT] (SEQ | OSL333 | AUGAAUGAAACAUAUACUACU[dT][dT] |
| A | ID NO: 553) | S | (SEQ ID NO: 927) |
| OSL334 | AAUCAGUAGUAUAUGUUUCAU[dT][dT] (SEQ | OSL334 | AUGAAACAUAUACUACUGAUU[dT][dT] |
| A | ID NO: 554) | S | (SEQ ID NO: 928) |
| OSL335 | AAAUAAUCAGUAGUAUAUGUU[dT][dT] (SEQ | OSL335 | AACAUAUACUACUGAUUAUUU[dT][dT] |
| A | ID NO: 555) | S | (SEQ ID NO: 929) |
| OSL336 | AAUCAAAGUAAUUACAGUCAG[dT][dT] (SEQ | OSL336 | CUGACUGUAAUUACUUUGAUU[dT][dT] |
| A | ID NO: 556) | S | (SEQ ID NO: 930) |
| OSL337 | AUCUAAUCAAAGUAAUUACAG[dT][dT] (SEQ | OSL337 | CUGUAAUUACUUUGAUUAGAU[dT][dT] |
| A | ID NO: 557) | S | (SEQ ID NO: 931) |
| OSL338 | UUAUUUCCAGUUGUUUAUCUA[dT][dT] (SEQ | OSL338 | UAGAUAAACAACUGGAAAUAA[dT][dT] |
| A | ID NO: 558) | S | (SEQ ID NO: 932) |
| OSL339 | UUAUUAGAACUUUUUCAGCAG[dT][dT] (SEQ | OSL339 | CUGCUGAAAAAGUUCUAAUAA[dT][dT] |
| A | ID NO: 559) | S | (SEQ ID NO: 933) |
| OSL340 | UUUAUUAGAACUUUUUCAGCA[dT][dT] (SEQ | OSL340 | UGCUGAAAAAGUUCUAAUAAA[dT][dT] |
| A | ID NO: 560) | S | (SEQ ID NO: 934) |

TABLE 5A
CD320 ANTISENSE TARGET

| | | | | | | SEQ |
|---|---|---|---|---|---|---|
| | target | start | | | | ID |
| ID | position | position | target sequence | Location | Size | NO: |
| OSC1 | 2-24 | 2 | TGCGCGTGCGCAGGGATAAGAGA | 5′ UTR | 21 | 996 |
| OSC2 | 4-26 | 4 | CGCGTGCGCAGGGATAAGAGAGC | 5′ UTR | 21 | 997 |
| OSC3 | 48-70 | 48 | GCGCCGCTGTGGGGACAGCATGA | 5′ UTR | 21 | 998 |
| OSC4 | 63-85 | 63 | CAGCATGAGCGGCGGTTGGATGG | 5′UTR- | 21 | 999 |
| | | | | CDS | | |
| OSC5 | 164-186 | 164 | CCGCCGCGAGCCCGCTTTCCACC | CDS | 21 | 1000 |
| OSC6 | 222-244 | 222 | CTCGTGCCCACCCACCAAGTTCC | CDS | 21 | 1001 |
| OSC7 | 225-247 | 225 | GTGCCCACCCACCAAGTTCCAGT | CDS | 21 | 1002 |
| OSC8 | 227-249 | 227 | GCCCACCCACCAAGTTCCAGTGC | CDS | 21 | 1003 |
| OSC9 | 244-266 | 244 | CAGTGCCGCACCAGTGGCTTATG | CDS | 21 | 1004 |
| OSC10 | 249-271 | 249 | CCGCACCAGTGGCTTATGCGTGC | CDS | 21 | 1005 |
| OSC11 | 282-304 | 282 | GCGCTGCGACAGGGACTTGGACT | CDS | 21 | 1006 |
| OSC12 | 306-328 | 306 | CAGCGATGGCAGCGATGAGGAGG | CDS | 21 | 1007 |
| OSC13 | 390-412 | 390 | CCCCTGCACCGGCGTCAGTGACT | CDS | 21 | 1008 |
| OSC14 | 411-433 | 411 | CTGCTCTGGGGGAACTGACAAGA | CDS | 21 | 1009 |
| OSC15 | 414-436 | 414 | CTCTGGGGGAACTGACAAGAAAC | CDS | 21 | 1010 |
| OSC16 | 417-439 | 417 | TGGGGGAACTGACAAGAAACTGC | CDS | 21 | 1011 |
| OSC17 | 422-466 | 422 | GAACTGACAAGAAACTGCGCAACTG | CDS | 25 | 1012 |
| OSC18 | 483-505 | 483 | CACGCTGAGCGATGACTGCATTC | CDS | 21 | 1013 |
| OSC19 | 484-506 | 484 | ACGCTGAGCGATGACTGCATTCC | CDS | 21 | 1014 |
| OSC20 | 487-509 | 487 | CTGAGCGATGACTGCATTCCACT | CDS | 21 | 1015 |
| OSC21 | 489-511 | 489 | GAGCGATGACTGCATTCCACTCA | CDS | 21 | 1016 |
| OSC22 | 520-542 | 520 | TGCGACGGCCACCCAGACTGTCC | CDS | 21 | 1017 |
| OSC23 | 556-578 | 556 | GAGCTCGGCTGTGGAACCAATGA | CDS | 21 | 1018 |
| OSC24 | 560-582 | 560 | TCGGCTGTGGAACCAATGAGATC | CDS | 21 | 1019 |
| OSC25 | 561-583 | 561 | CGGCTGTGGAACCAATGAGATCC | CDS | 21 | 1020 |
| OSC26 | 564-586 | 564 | CTGTGGAACCAATGAGATCCTCC | CDS | 21 | 1021 |
| OSC27 | 626-648 | 626 | TGGAGAGTGTCACCTCTCTCAGG | CDS | 21 | 1022 |
| OSC28 | 641-663 | 641 | CTCTCAGGAATGCCACAACCATG | CDS | 21 | 1023 |
| OSC29 | 689-711 | 689 | TCCCCTCTGTCGGGAATGCCACA | CDS | 21 | 1024 |
| OSC30 | 695-717 | 695 | CTGTCGGGAATGCCACATCCTCC | CDS | 21 | 1025 |
| OSC31 | 719-741 | 719 | CTGCCGGAGACCAGTCTGGAAGC | CDS | 21 | 1026 |
| OSC32 | 741-763 | 741 | CCCAACTGCCTATGGGGTTATTG | CDS | 21 | 1027 |
| OSC33 | 767-789 | 767 | CTGCTGCGGTGCTCAGTGCAAGC | CDS | 21 | 1028 |
| OSC34 | 795-817 | 795 | CACCGCCACCCTCCTCCTTTTGT | CDS | 21 | 1029 |
| OSC35 | 797-819 | 797 | CCGCCACCCTCCTCCTTTTGTCC | CDS | 21 | 1030 |
| OSC36 | 843-865 | 843 | CCGCCCACTGGGGTTACTGGTGG | CDS | 21 | 1031 |
| OSC37 | 852-874 | 852 | GGGGTTACTGGTGGCCATGAAGG | CDS | 21 | 1032 |
| OSC38 | 857-879 | 857 | TACTGGTGGCCATGAAGGAGTCC | CDS | 21 | 1033 |
| OSC39 | 874-896 | 874 | GAGTCCCTGCTGCTGTCAGAACA | CDS | 21 | 1034 |
| OSC40 | 878-900 | 878 | CCCTGCTGCTGTCAGAACAGAAG | CDS | 21 | 1035 |
| OSC41 | 881-903 | 881 | TGCTGCTGTCAGAACAGAAGACC | CDS | 21 | 1036 |
| OSC42 | 884-906 | 884 | TGCTGTCAGAACAGAAGACCTCG | CDS | 21 | 1037 |
| OSC43 | 901-923 | 901 | ACCTCGCTGCCCTGAGGACAAGC | CDS | 21 | 1038 |
| OSC44 | 907-929 | 907 | CTGCCCTGAGGACAAGCACTTGC | CDS- | 21 | 1039 |
| | | | | 3′UTR | | |
| OSC45 | 971-993 | 971 | GAGCAGTGATGCGGATGGGTACC | 3′ UTR | 21 | 1040 |
| OSC46 | 995-1017 | 995 | GGGCACACCAGCCCTCAGAGACC | 3′ UTR | 21 | 1041 |
| OSC47 | 1006-1026 | 1006 | CCCTCAGAGACCTGAGCTCTT | 3′ UTR | 21 | 1393 |
| OSC48 | 1006-1028 | 1006 | CCCTCAGAGACCTGAGCTCTTCT | 3′ UTR | 21 | 1042 |
| OSC49 | 1008-1030 | 1008 | CTCAGAGACCTGAGCTCTTCTGG | 3′ UTR | 21 | 1043 |
| OSC50 | 1082-1104 | 1082 | GGGTCCCTGGACACTCCCTATGG | 3′ UTR | 21 | 1044 |
| OSC51 | 1085-1107 | 1085 | TCCCTGGACACTCCCTATGGAGA | 3′ UTR | 21 | 1045 |
| OSC52 | 1088-1110 | 1088 | CTGGACACTCCCTATGGAGATCC | 3′ UTR | 21 | 1046 |
| OSC53 | 1129-1151 | 1129 | ACCTGCCACAGCCAGAACTGAGG | 3′ UTR | 21 | 1047 |
| OSC54 | 1163-1185 | 1163 | GGCAGCTCCCAGGGGGTAGAACG | 3′ UTR | 21 | 1048 |
| OSC55 | 1176-1198 | 1176 | GGGTAGAACGGCCCTGTGCTTAA | 3′ UTR | 21 | 1049 |
| OSC56 | 1182-1204 | 1182 | AACGGCCCTGTGCTTAAGACACT | 3′ UTR | 21 | 1050 |
| OSC57 | 1184-1206 | 1184 | CGGCCCTGTGCTTAAGACACTCC | 3′ UTR | 21 | 1051 |
| OSC58 | 1237-1259 | 1237 | TTGCTTCACATCCTCAAAAAAAA | 3′ UTR | 21 | 1052 |
| OSC59 | 1238-1260 | 1238 | TGCTTCACATCCTCAAAAAAAAA | 3′ UTR | 21 | 1053 |

TABLE 6A
LRP2 ANTISENSE TARGET

| | | | | | | SEQ |
|---|---|---|---|---|---|---|
| | Target | Start | | | | ID |
| ID | position | position | Target sequence | Location | Size | NO: |
| OSL1 | 512-534 | 512 | GTCAAGATTGCTCACAAAGTACA | CDS | 21 | 1054 |
| OSL2 | 566-588 | 566 | GTCAGTGTATCCCAAGTGAATAC | CDS | 21 | 1055 |
| OSL3 | 763-785 | 763 | TTGCACAATGAGTTTTCATGTGG | CDS | 21 | 1056 |
| OSL4 | 939-961 | 939 | TGGAGAAGATGACTGTAAAGATA | CDS | 21 | 1057 |
| OSL5 | 941-963 | 941 | GAGAAGATGACTGTAAAGATAAT | CDS | 21 | 1058 |
| OSL6 | 992-1014 | 992 | CTCATGATGTTCATAAATGTTCC | CDS | 21 | 1059 |
| OSL7 | 1053- | 1053 | CTCCATTTATAAAGTTTGTGATG | CDS | 21 | 1060 |
| | 1075 | | | | | |
| OSL8 | 1054- | 1054 | TCCATTTATAAAGTTTGTGATGG | CDS | 21 | 1061 |
| | 1076 | | | | | |
| OSL9 | 1119- | 1119 | TACCGGAAAATACTGTAGTATGA | CDS | 21 | 1062 |
| | 1141 | | | | | |
| OSL10 | 1121- | 1121 | CCGGAAAATACTGTAGTATGACT | CDS | 21 | 1063 |
| | 1143 | | | | | |
| OSL11 | 1267- | 1267 | TGCCAGATATGGGGAATTTGTGA | CDS | 21 | 1064 |
| | 1289 | | | | | |
| OSL12 | 1329- | 1329 | CTGTGAAGAAGGGTATATCTTGG | CDS | 21 | 1065 |
| | 1351 | | | | | |
| OSL13 | 1356- | 1356 | TGGACAGTATTGCAAAGCTAATG | CDS | 21 | 1066 |
| | 1378 | | | | | |
| OSL14 | 1360- | 1360 | CAGTATTGCAAAGCTAATGATTC | CDS | 21 | 1067 |
| | 1382 | | | | | |
| OSL15 | 1366- | 1366 | TGCAAAGCTAATGATTCCTTTGG | CDS | 21 | 1068 |
| | 1388 | | | | | |
| OSL16 | 1423- | 1423 | TTGTTAATTGGTGATATTCATGG | CDS | 21 | 1069 |
| | 1445 | | | | | |
| OSL17 | 1541- | 1541 | CCGTGCAAAATAAGGTTTTTTCA | CDS | 21 | 1070 |
| | 1563 | | | | | |
| OSL18 | 1543- | 1543 | GTGCAAAATAAGGTTTTTTCAGT | CDS | 21 | 1071 |
| | 1565 | | | | | |
| OSL19 | 1552- | 1552 | AAGGTTTTTTCAGTTGACATTAA | CDS | 21 | 1072 |
| | 1574 | | | | | |
| OSL20 | 1553- | 1553 | AGGTTTTTTCAGTTGACATTAAT | CDS | 21 | 1073 |
| | 1575 | | | | | |
| OSL21 | 1562- | 1562 | CAGTTGACATTAATGGTTTAAAT | CDS | 21 | 1074 |
| | 1584 | | | | | |
| OSL22 | 1565- | 1565 | TTGACATTAATGGTTTAAATATC | CDS | 21 | 1075 |
| | 1587 | | | | | |
| OSL23 | 1638- | 1638 | CTGGGTTAATAATAAAATCTATC | CDS | 21 | 1076 |
| | 1660 | | | | | |
| OSL24 | 1639- | 1639 | TGGGTTAATAATAAAATCTATCT | CDS | 21 | 1077 |
| | 1661 | | | | | |
| OSL25 | 1680- | 1680 | CCGCATAGATATGGTAAATTTGG | CDS | 21 | 1078 |
| | 1702 | | | | | |
| OSL26 | 1719- | 1719 | TACCCTTATAACTGAAAACTTGG | CDS | 21 | 1079 |
| | 1741 | | | | | |
| OSL27 | 1767- | 1767 | CCCAACTGTTGGTTATTTATTTT | CDS | 21 | 1080 |
| | 1789 | | | | | |
| OSL28 | 1772- | 1772 | CTGTTGGTTATTTATTTTTCTCA | CDS | 21 | 1081 |
| | 1794 | | | | | |
| OSL29 | 1895- | 1895 | GGGTAACTCTGGATATGATATCG | CDS | 21 | 1082 |
| | 1917 | | | | | |
| OSL30 | 1942- | 1942 | CGGTTTGATTACATTGAAACTGT | CDS | 21 | 1083 |
| | 1964 | | | | | |
| OSL31 | 1946- | 1946 | TTGATTACATTGAAACTGTAACT | CDS | 21 | 1084 |
| | 1968 | | | | | |
| OSL32 | 1951- | 1951 | TACATTGAAACTGTAACTTATGA | CDS | 21 | 1085 |
| | 1973 | | | | | |
| OSL33 | 2187- | 2187 | TGCTACCAATCCGTGTAAAGATA | CDS | 21 | 1086 |
| | 2209 | | | | | |
| OSL34 | 2437- | 2437 | TTCTTTGTCGGGATTGATTTTGA | CDS | 21 | 1087 |
| | 2459 | | | | | |
| OSL35 | 2469- | 2469 | CAGCACTATCTTTTTTTCAGATA | CDS | 21 | 1088 |
| | 2491 | | | | | |
| OSL36 | 2470- | 2470 | AGCACTATCTTTTTTTCAGATAT | CDS | 21 | 1089 |
| | 2492 | | | | | |
| OSL37 | 2491- | 2491 | ATGTCAAAACACATGATTTTTAA | CDS | 21 | 1090 |
| | 2513 | | | | | |
| OSL38 | 2498- | 2498 | AACACATGATTTTTAAGCAAAAG | CDS | 21 | 1091 |
| | 2520 | | | | | |
| OSL39 | 2558- | 2558 | GGGTGGAAAATGTTGAAAGTTTG | CDS | 21 | 1092 |
| | 2580 | | | | | |
| OSL40 | 2579- | 2579 | TGGCTTTTGATTGGATTTCAAAG | CDS | 21 | 1093 |
| | 2601 | | | | | |
| OSL41 | 2580- | 2580 | GGCTTTTGATTGGATTTCAAAGA | CDS | 21 | 1094 |
| | 2602 | | | | | |
| OSL42 | 2589- | 2589 | TTGGATTTCAAAGAATCTCTATT | CDS | 21 | 1095 |
| | 2611 | | | | | |
| OSL43 | 2590- | 2590 | TGGATTTCAAAGAATCTCTATTG | CDS | 21 | 1096 |
| | 2612 | | | | | |
| OSL44 | 2670- | 2670 | CACAGTAGTTCAGTATTTAAATA | CDS | 21 | 1097 |
| | 2692 | | | | | |
| OSL45 | 2672- | 2672 | CAGTAGTTCAGTATTTAAATAAC | CDS | 21 | 1098 |
| | 2694 | | | | | |
| OSL46 | 2714- | 2714 | ATCCTTTTGCCGGGTATCTATTC | CDS | 21 | 1099 |
| | 2736 | | | | | |
| OSL47 | 2800- | 2800 | CCTGTAATAAACACTACTCTT | CDS | 21 | 1394 |
| | 2820 | | | | | |
| OSL48 | 2869- | 2869 | TGGGTAGATGCCTATTTTGATAA | CDS | 21 | 1100 |
| | 2891 | | | | | |
| OSL49 | 2877- | 2877 | TGCCTATTTTGATAAAATTGAGC | CDS | 21 | 1101 |
| | 2899 | | | | | |
| OSL50 | 2971- | 2971 | GCCATCTTTGGAGAGCATTTATT | CDS | 21 | 1102 |
| | 2993 | | | | | |
| OSL51 | 3074- | 3074 | TTGCTTACATACTGCATTTGAAA | CDS | 21 | 1103 |
| | 3096 | | | | | |
| OSL52 | 3075- | 3075 | TGCTTACATACTGCATTTGAAAT | CDS | 21 | 1104 |
| | 3097 | | | | | |
| OSL53 | 3120- | 3120 | TGGTTCTAACGCCTGTAATCAAC | CDS | 21 | 1105 |
| | 3142 | | | | | |
| OSL54 | 3356- | 3356 | TCGATGATTGTCATGATAACAGT | CDS | 21 | 1106 |
| | 3378 | | | | | |
| OSL55 | 3546- | 3546 | CACCCAATACACCTGTGATAATC | CDS | 21 | 1107 |
| | 3568 | | | | | |
| OSL56 | 3547- | 3547 | ACCCAATACACCTGTGATAATCA | CDS | 21 | 1108 |
| | 3569 | | | | | |
| OSL57 | 3569- | 3569 | ACCAGTGTATCTCAAAGAACTGG | CDS | 21 | 1109 |
| | 3591 | | | | | |
| OSL58 | 3629- | 3629 | ATGAAAAGAACTGCAATTCGACA | CDS | 21 | 1110 |
| | 3651 | | | | | |
| OSL59 | 3681- | 3681 | CCCCAATCATCGATGTATTGACC | CDS | 21 | 1111 |
| | 3703 | | | | | |
| OSL60 | 3690- | 3690 | TCGATGTATTGACCTATCGTTTG | CDS | 21 | 1112 |
| | 3712 | | | | | |
| OSL61 | 3693- | 3693 | ATGTATTGACCTATCGTTTGTCT | CDS | 21 | 1113 |
| | 3715 | | | | | |
| OSL62 | 3828- | 3828 | TCGTTGTGATGGTGTTTTTGATT | CDS | 21 | 1114 |
| | 3850 | | | | | |
| OSL63 | 3945- | 3945 | CCCGAACTTCTGGGAATGTGATG | CDS | 21 | 1115 |
| | 3967 | | | | | |
| OSL64 | 3946- | 3946 | CCGAACTTCTGGGAATGTGATGG | CDS | 21 | 1116 |
| | 3968 | | | | | |
| OSL65 | 4015- | 4015 | CCCAAGACTTGCCCTTCATCATA | CDS | 21 | 1117 |
| | 4037 | | | | | |
| OSL66 | 4348- | 4348 | TTCTTACTTGCCAATGATTCTAA | CDS | 21 | 1118 |
| | 4370 | | | | | |
| OSL67 | 4379- | 4379 | AAGACATAGATGAATGTGATATT | CDS | 21 | 1119 |
| | 4401 | | | | | |
| OSL68 | 4381- | 4381 | GACATAGATGAATGTGATATTCT | CDS | 21 | 1120 |
| | 4403 | | | | | |
| OSL69 | 4455- | 4455 | GTGTGATACAGGCTACATGTTAG | CDS | 21 | 1121 |
| | 4477 | | | | | |
| OSL70 | 4464- | 4464 | AGGCTACATGTTAGAAAGTGATG | CDS | 21 | 1122 |
| | 4486 | | | | | |
| OSL71 | 4465- | 4465 | GGCTACATGTTAGAAAGTGATGG | CDS | 21 | 1123 |
| | 4487 | | | | | |
| OSL72 | 4597- | 4597 | GTCGAGAATGGTTCTTACATTGT | CDS | 21 | 1124 |
| | 4619 | | | | | |
| OSL73 | 4600- | 4600 | GAGAATGGTTCTTACATTGTAGC | CDS | 21 | 1125 |
| | 4622 | | | | | |
| OSL74 | 4612- | 4612 | TACATTGTAGCTGTTGATTTTGA | CDS | 21 | 1126 |
| | 4634 | | | | | |
| OSL75 | 4620- | 4620 | AGCTGTTGATTTTGATTCAATTA | CDS | 21 | 1127 |
| | 4642 | | | | | |
| OSL76 | 4622- | 4622 | CTGTTGATTTTGATTCAATTAGT | CDS | 21 | 1128 |
| | 4644 | | | | | |
| OSL77 | 4635- | 4635 | TTCAATTAGTGGTCGTATCTTTT | CDS | 21 | 1129 |
| | 4657 | | | | | |
| OSL78 | 4732- | 4732 | AGCATCATCTTGACTGAAACTAT | CDS | 21 | 1130 |
| | 4754 | | | | | |
| OSL79 | 4735- | 4735 | ATCATCTTGACTGAAACTATTGC | CDS | 21 | 1131 |
| | 4757 | | | | | |
| OSL80 | 4741- | 4741 | TTGACTGAAACTATTGCAATAGA | CDS | 21 | 1132 |
| | 4763 | | | | | |
| OSL81 | 4743- | 4743 | GACTGAAACTATTGCAATAGATT | CDS | 21 | 1133 |
| | 4765 | | | | | |
| OSL82 | 4745- | 4745 | CTGAAACTATTGCAATAGATTGG | CDS | 21 | 1134 |
| | 4767 | | | | | |
| OSL83 | 4806- | 4806 | AACAATTGAAGTCTCCAAAATTG | CDS | 21 | 1135 |
| | 4828 | | | | | |
| OSL84 | 4847- | 4847 | TGCTGATTAGTAAAAACCTAACA | CDS | 21 | 1136 |
| | 4869 | | | | | |
| OSL85 | 4883- | 4883 | TAGCATTAGATCCCAGAATGAAT | CDS | 21 | 1137 |
| | 4905 | | | | | |
| OSL86 | 4884- | 4884 | AGCATTAGATCCCAGAATGAATG | CDS | 21 | 1138 |
| | 4906 | | | | | |
| OSL87 | 4896- | 4896 | CAGAATGAATGAGCATCTACTGT | CDS | 21 | 1139 |
| | 4918 | | | | | |
| OSL88 | 5077- | 5077 | ATGGACTTTTGTGATTATAATGG | CDS | 21 | 1140 |
| | 5099 | | | | | |
| OSL89 | 5080- | 5080 | GACTTTTGTGATTATAATGGACA | CDS | 21 | 1141 |
| | 5102 | | | | | |
| OSL90 | 5126- | 5126 | GTGATTTGATTATACGGCA | CDS | 19 | 1142 |
| | 5144 | | | | | |
| OSL91 | 5241- | 5241 | GTCAGTTGTAATGTATAATATTC | CDS | 21 | 1143 |
| | 5263 | | | | | |
| OSL92 | 5246- | 5246 | TTGTAATGTATAATATTCAATGG | CDS | 21 | 1144 |
| | 5268 | | | | | |
| OSL93 | 5291- | 5291 | ATCCTTCGAAACAACCAAATTCC | CDS | 21 | 1145 |
| | 5313 | | | | | |
| OSL94 | 5295- | 5295 | TTCGAAACAACCAAATTCCGTGA | CDS | 21 | 1146 |
| | 5317 | | | | | |
| OSL95 | 5447- | 5447 | AACCTTTCTTAATAACTGTAAGG | CDS | 21 | 1147 |
| | 5469 | | | | | |
| OSL96 | 5467- | 5467 | AGGCAACATATAATTTTTGGAAT | CDS | 21 | 1148 |
| | 5489 | | | | | |
| OSL97 | 5468- | 5468 | GGCAACATATAATTTTTGGAATC | CDS | 21 | 1149 |
| | 5490 | | | | | |
| OSL98 | 5538- | 5538 | AGGGATACAGAATGGTTTAGATG | CDS | 21 | 1150 |
| | 5560 | | | | | |
| OSL99 | 5539- | 5539 | GGGATACAGAATGGTTTAGATGT | CDS | 21 | 1151 |
| | 5561 | | | | | |
| OSL100 | 5545- | 5545 | CAGAATGGTTTAGATGTTGAATT | CDS | 21 | 1152 |
| | 5567 | | | | | |
| OSL101 | 5584- | 5584 | TACATCTATTGGGTTGAAAATCC | CDS | 21 | 1153 |
| | 5606 | | | | | |
| OSL102 | 5644- | 5644 | AGGACAGTATTTGCTTCTATATC | CDS | 21 | 1154 |
| | 5666 | | | | | |
| OSL103 | 5648- | 5648 | CAGTATTTGCTTCTATATCTATG | CDS | 21 | 1155 |
| | 5670 | | | | | |
| OSL104 | 5677- | 5677 | CCTTCTATGAACCTGGCCTTA | CDS | 21 | 1156 |
| | 5697 | | | | | |
| OSL105 | 5692- | 5692 | GCCTTAGATTGGATTTCAAGAAA | CDS | 21 | 1157 |
| | 5714 | | | | | |
| OSL106 | 5701- | 5701 | TGGATTTCAAGAAACCTTTATTC | CDS | 21 | 1158 |
| | 5723 | | | | | |
| OSL107 | 5738- | 5738 | CTCAGTCAATCGAGGTTTTGACA | CDS | 21 | 1159 |
| | 5760 | | | | | |
| OSL108 | 5765- | 5765 | ACGGAGATATCAGATACAGAAAA | CDS | 21 | 1160 |
| | 5787 | | | | | |
| OSL109 | 5766- | 5766 | CGGAGATATCAGATACAGAAAAA | CDS | 21 | 1161 |
| | 5788 | | | | | |
| OSL110 | 5768- | 5768 | GAGATATCAGATACAGAAAAACA | CDS | 21 | 1162 |
| | 5790 | | | | | |
| OSL111 | 5775- | 5775 | CAGATACAGAAAAACATTGATTG | CDS | 21 | 1163 |
| | 5797 | | | | | |
| OSL112 | 6115- | 6115 | GTCCATGATTCTTTCCTTTATTA | CDS | 21 | 1164 |
| | 6137 | | | | | |
| OSL113 | 6116- | 6116 | TCCATGATTCTTTCCTTTATTAT | CDS | 21 | 1165 |
| | 6138 | | | | | |
| OSL114 | 6123- | 6123 | TTCTTTCCTTTATTATACTGATG | CDS | 21 | 1166 |
| | 6145 | | | | | |
| OSL115 | 6146- | 6146 | AACAGTATGAGGTCATTGAAAGA | CDS | 21 | 1167 |
| | 6168 | | | | | |
| OSL116 | 6202- | 6202 | TTGAGAGATAATGTTCCAAATCT | CDS | 21 | 1168 |
| | 6224 | | | | | |
| OSL117 | 6204- | 6204 | GAGAGATAATGTTCCAAATCTGA | CDS | 21 | 1169 |
| | 6226 | | | | | |
| OSL118 | 6206- | 6206 | GAGATAATGTTCCAAATCTGAGG | CDS | 21 | 1170 |
| | 6228 | | | | | |
| OSL119 | 6266- | 6266 | CCTCAAATGGCTGTAGCAA | CDS | 19 | 1171 |
| | 6284 | | | | | |
| OSL120 | 6387- | 6387 | CTCTCCATATAACTCTTTCATTG | CDS | 21 | 1172 |
| | 6409 | | | | | |
| OSL121 | 6390- | 6390 | TCCATATAACTCTTTCATTGTTG | CDS | 21 | 1173 |
| | 6412 | | | | | |
| OSL122 | 6397- | 6397 | AACTCTTTCATTGTTGTTTCAAT | CDS | 21 | 1174 |
| | 6419 | | | | | |
| OSL123 | 6399- | 6399 | CTCTTTCATTGTTGTTTCAATGC | CDS | 21 | 1175 |
| | 6421 | | | | | |
| OSL124 | 6425- | 6425 | CTGCAATCAGAGGCTTTAGCTTG | CDS | 21 | 1176 |
| | 6447 | | | | | |
| OSL125 | 6426- | 6426 | TGCAATCAGAGGCTTTAGCTTGG | CDS | 21 | 1177 |
| | 6448 | | | | | |
| OSL126 | 6434- | 6434 | GAGGCTTTAGCTTGGAATTGTCA | CDS | 21 | 1178 |
| | 6456 | | | | | |
| OSL127 | 6436- | 6436 | GGCTTTAGCTTGGAATTGTCAGA | CDS | 21 | 1179 |
| | 6458 | | | | | |
| OSL128 | 6445- | 6445 | TTGGAATTGTCAGATCATTCAGA | CDS | 21 | 1180 |
| | 6467 | | | | | |
| OSL129 | 6603- | 6603 | TGGATCTTCTCTGATGAACATTG | CDS | 21 | 1181 |
| | 6625 | | | | | |
| OSL130 | 6619- | 6619 | AACATTGTGACACATGGAATAGG | CDS | 21 | 1182 |
| | 6641 | | | | | |
| OSL131 | 6711- | 6711 | TTCTGAAACACTGATAGAAGTTC | CDS | 21 | 1183 |
| | 6733 | | | | | |
| OSL132 | 6725- | 6725 | TAGAAGTTCTGCGGATCAATACT | CDS | 21 | 1184 |
| | 6747 | | | | | |
| OSL133 | 6797- | 6797 | TTGTTGTAGATCCCAAGAACAGA | CDS | 21 | 1185 |
| | 6819 | | | | | |
| OSL134 | 6849- | 6849 | ACCAAAGATTGAGCGTTCTTTCC | CDS | 21 | 1186 |
| | 6871 | | | | | |
| OSL135 | 6939- | 6939 | CCGAAGTGATGGCTACGTTTATT | CDS | 21 | 1187 |
| | 6961 | | | | | |
| OSL136 | 6961- | 6961 | TGGGTTGATGATTCTTTAGATAT | CDS | 21 | 1188 |
| | 6983 | | | | | |
| OSL137 | 6965- | 6965 | TTGATGATTCTTTAGATATAATT | CDS | 21 | 1189 |
| | 6987 | | | | | |
| OSL138 | 7062- | 7062 | CACTGTTTTTGAAAATTCTATCA | CDS | 21 | 1190 |
| | 7084 | | | | | |
| OSL139 | 7064- | 7064 | CTGTTTTTGAAAATTCTATCATA | CDS | 21 | 1191 |
| | 7086 | | | | | |
| OSL140 | 7087- | 7087 | TGGGTAGATAGGAATTTGAAAAA | CDS | 21 | 1192 |
| | 7109 | | | | | |
| OSL141 | 7088- | 7088 | GGGTAGATAGGAATTTGAAAAAG | CDS | 21 | 1193 |
| | 7110 | | | | | |
| OSL142 | 7152- | 7152 | CACAGTGATAAGAGACAATATCA | CDS | 21 | 1194 |
| | 7174 | | | | | |
| OSL143 | 7154- | 7154 | CAGTGATAAGAGACAATATCAAC | CDS | 21 | 1195 |
| | 7176 | | | | | |
| OSL144 | 7348- | 7348 | GGCAAGAATTGTGCCATTTCAAC | CDS | 21 | 1196 |
| | 7370 | | | | | |
| OSL145 | 7351- | 7351 | AAGAATTGTGCCATTTCAACAGA | CDS | 21 | 1197 |
| | 7373 | | | | | |
| OSL146 | 7358- | 7358 | GTGCCATTTCAACAGAAAATTTC | CDS | 21 | 1198 |
| | 7380 | | | | | |
| OSL147 | 7359- | 7359 | TGCCATTTCAACAGAAAATTTCC | CDS | 21 | 1199 |
| | 7381 | | | | | |
| OSL148 | 7381- | 7381 | CTCATCTTTGCCTTGTCTAATTC | CDS | 21 | 1200 |
| | 7403 | | | | | |
| OSL149 | 7443- | 7443 | ACCTTTCCAAACAATAAATGTGG | CDS | 21 | 1201 |
| | 7465 | | | | | |
| OSL150 | 7486- | 7486 | GACTATGACAGTGTAAGTGATAG | CDS | 21 | 1202 |
| | 7508 | | | | | |
| OSL151 | 7494- | 7494 | CAGTGTAAGTGATAGAATCTACT | CDS | 21 | 1203 |
| | 7516 | | | | | |
| OSL152 | 7496- | 7496 | GTGTAAGTGATAGAATCTACTTC | CDS | 21 | 1204 |
| | 7518 | | | | | |
| OSL153 | 7506- | 7506 | TAGAATCTACTTCACACAAAATT | CDS | 21 | 1205 |
| | 7528 | | | | | |
| OSL154 | 7510- | 7510 | ATCTACTTCACACAAAATTTAGC | CDS | 21 | 1206 |
| | 7532 | | | | | |
| OSL155 | 7627- | 7627 | GCCTTTGACTGGATTACTAGAAG | CDS | 21 | 1207 |
| | 7649 | | | | | |
| OSL156 | 7633- | 7633 | GACTGGATTACTAGAAGAATTTA | CDS | 21 | 1208 |
| | 7655 | | | | | |
| OSL157 | 7635- | 7635 | CTGGATTACTAGAAGAATTTATT | CDS | 21 | 1209 |
| | 7657 | | | | | |
| OSL158 | 7636- | 7636 | TGGATTACTAGAAGAATTTATTA | CDS | 21 | 1210 |
| | 7658 | | | | | |
| OSL159 | 8007- | 8007 | GACTCTCTATGGCCAGTATATTT | CDS | 21 | 1211 |
| | 8029 | | | | | |
| OSL160 | 8036- | 8036 | CTGACTTGTACACACAAAGAATT | CDS | 21 | 1212 |
| | 8058 | | | | | |
| OSL161 | 8038- | 8038 | GACTTGTACACACAAAGAATTTA | CDS | 21 | 1213 |
| | 8060 | | | | | |
| OSL162 | 8044- | 8044 | TACACACAAAGAATTTACCGAGC | CDS | 21 | 1214 |
| | 8066 | | | | | |
| OSL163 | 8150- | 8150 | ACCAGAAACAACAGTGTAACAAT | CDS | 21 | 1215 |
| | 8172 | | | | | |
| OSL164 | 8152- | 8152 | CAGAAACAACAGTGTAACAATCC | CDS | 21 | 1216 |
| | 8174 | | | | | |
| OSL165 | 8167- | 8167 | AACAATCCTTGTGAACAGTTTAA | CDS | 21 | 1217 |
| | 8189 | | | | | |
| OSL166 | 8293- | 8293 | GTGGACAATGGTGAACGATGTGG | CDS | 21 | 1218 |
| | 8315 | | | | | |
| OSL167 | 8564- | 8564 | CGGAGTTTATGTGCAATAACAGA | CDS | 21 | 1219 |
| | 8586 | | | | | |
| OSL168 | 8566- | 8566 | GAGTTTATGTGCAATAACAGAAG | CDS | 21 | 1220 |
| | 8588 | | | | | |
| OSL169 | 8685- | 8685 | TGGATACACAAAATGTCATAATT | CDS | 21 | 1221 |
| | 8707 | | | | | |
| OSL170 | 8689- | 8689 | TACACAAAATGTCATAATTCAAA | CDS | 21 | 1222 |
| | 8711 | | | | | |
| OSL171 | 8691- | 8691 | CACAAAATGTCATAATTCAAATA | CDS | 21 | 1223 |
| | 8713 | | | | | |
| OSL172 | 8699- | 8699 | GTCATAATTCAAATATTTGTATT | CDS | 21 | 1224 |
| | 8721 | | | | | |
| OSL173 | 8715- | 8715 | TTGTATTCCTCGCGTTTATTTGT | CDS | 21 | 1225 |
| | 8737 | | | | | |
| OSL174 | 8768- | 8768 | GTGATGAAAACCCTACTTATTGC | CDS | 21 | 1226 |
| | 8790 | | | | | |
| OSL175 | 8844- | 8844 | TCCTCAACATTGGTATTGTGATC | CDS | 21 | 1227 |
| | 8866 | | | | | |
| OSL176 | 8854- | 8854 | TGGTATTGTGATCAAGAAACAGA | CDS | 21 | 1228 |
| | 8876 | | | | | |
| OSL177 | 8861- | 8861 | GTGATCAAGAAACAGATTGTTTT | CDS | 21 | 1229 |
| | 8883 | | | | | |
| OSL178 | 9063- | 9063 | TTCCGAGTTTCTCTGTGTAAATG | CDS | 21 | 1230 |
| | 9085 | | | | | |
| OSL179 | 9064- | 9064 | TCCGAGTTTCTCTGTGTAAATGA | CDS | 21 | 1231 |
| | 9086 | | | | | |
| OSL180 | 9153- | 9153 | CGGCTACGATGAGAATCAGAATT | CDS | 21 | 1232 |
| | 9175 | | | | | |
| OSL181 | 9181- | 9181 | AGGAGAACTTGCTCTGAAAATGA | CDS | 21 | 1233 |
| | 9203 | | | | | |
| OSL182 | 9221- | 9221 | GACTGTGTATCCCAAAGATATTC | CDS | 21 | 1234 |
| | 9243 | | | | | |
| OSL183 | 9308- | 9308 | GCCAACAGAATCAGTTTACCTGT | CDS | 21 | 1235 |
| | 9330 | | | | | |
| OSL184 | 9595- | 9595 | GACACCTTAACCAGTTTCTATTG | CDS | 21 | 1236 |
| | 9617 | | | | | |
| OSL185 | 9598- | 9598 | ACCTTAACCAGTTTCTATTGTTC | CDS | 21 | 1237 |
| | 9620 | | | | | |
| OSL186 | 9657- | 9657 | GACTTGTGTTGATATTGATGAAT | CDS | 21 | 1238 |
| | 9679 | | | | | |
| OSL187 | 9719- | 9719 | ATGTAATAGGCTCCTACATCTGT | CDS | 21 | 1239 |
| | 9741 | | | | | |
| OSL188 | 9786- | 9786 | CCGGCAAAACAGTAACATCGAAC | CDS | 21 | 1240 |
| | 9808 | | | | | |
| OSL189 | 9807- | 9807 | ACCCTATCTCATTTTTAGCAACC | CDS | 21 | 1241 |
| | 9829 | | | | | |
| OSL190 | 9826- | 9826 | AACCGTTACTATTTGAGAAATTT | CDS | 21 | 1242 |
| | 9848 | | | | | |
| OSL191 | 9827- | 9827 | ACCGTTACTATTTGAGAAATTTA | CDS | 21 | 1243 |
| | 9849 | | | | | |
| OSL192 | 9828- | 9828 | CCGTTACTATTTGAGAAATTTAA | CDS | 21 | 1244 |
| | 9850 | | | | | |
| OSL193 | 9832- | 9832 | TACTATTTGAGAAATTTAACTAT | CDS | 21 | 1245 |
| | 9854 | | | | | |
| OSL194 | 9838- | 9838 | TTGAGAAATTTAACTATAGATGG | CDS | 21 | 1246 |
| | 9860 | | | | | |
| OSL195 | 9849- | 9849 | AACTATAGATGGCTATTTTTACT | CDS | 21 | 1247 |
| | 9871 | | | | | |
| OSL196 | 9892- | 9892 | GACAATGTTGTGGCATTAGATTT | CDS | 21 | 1248 |
| | 9914 | | | | | |
| OSL197 | 9925- | 9925 | GAGAAGAGATTGTATTGGATTGA | CDS | 21 | 1249 |
| | 9947 | | | | | |
| OSL198 | 9956- | 9956 | GGCAAGTCATTGAGAGAATGTTT | CDS | 21 | 1250 |
| | 9978 | | | | | |
| OSL199 | 9987- | 9987 | GACAAACAAGGAGACAATCATAA | CDS | 21 | 1251 |
| | 10009 | | | | | |
| OSL200 | 10272- | 10272 | AACCAACAAGTCTGTGATAATCT | CDS | 21 | 1252 |
| | 10294 | | | | | |
| OSL201 | 10444- | 10444 | TTCGCTATTACCATTTTTGAAGA | CDS | 21 | 1253 |
| | 10466 | | | | | |
| OSL202 | 10446- | 10446 | CGCTATTACCATTTTTGAAGACA | CDS | 21 | 1254 |
| | 10468 | | | | | |
| OSL203 | 10499- | 10499 | CAGTGGAAAAGGGAAACAAATAT | CDS | 21 | 1255 |
| | 10521 | | | | | |
| OSL204 | 10513- | 10513 | AACAAATATGATGGATCAAATAG | CDS | 21 | 1256 |
| | 10535 | | | | | |
| OSL205 | 10574- | 10574 | TCCATGTGTACCATCCATATAGG | CDS | 21 | 1257 |
| | 10596 | | | | | |
| OSL206 | 10958- | 10958 | CTGATGAAGACCGTCTTCTTTGT | CDS | 21 | 1258 |
| | 10980 | | | | | |
| OSL207 | 11246- | 11246 | GTGACAACTTCACAGAATTCAGC | CDS | 21 | 1259 |
| | 11268 | | | | | |
| OSL208 | 11623- | 11623 | CAGTGTACAAGTGGACATTGTGT | CDS | 21 | 1260 |
| | 11645 | | | | | |
| OSL209 | 11625- | 11625 | GTGTACAAGTGGACATTGTGTAC | CDS | 21 | 1261 |
| | 11647 | | | | | |
| OSL210 | 11745- | 11745 | TACTATGTTCGAATGCAAAAACC | CDS | 21 | 1262 |
| | 11767 | | | | | |
| OSL211 | 11749- | 11749 | ATGTTCGAATGCAAAAACCATGT | CDS | 21 | 1263 |
| | 11771 | | | | | |
| OSL212 | 11757- | 11757 | ATGCAAAAACCATGTTTGTATCC | CDS | 21 | 1264 |
| | 11779 | | | | | |
| OSL213 | 11779- | 11779 | CCGCCATATTGGAAATGTGATGG | CDS | 21 | 1265 |
| | 11801 | | | | | |
| OSL214 | 11820- | 11820 | TGGTTCAGATGAAGAACTTCACC | CDS | 21 | 1266 |
| | 11842 | | | | | |
| OSL215 | 11887- | 11887 | GACAACAATCGCTGCATTTATAG | CDS | 21 | 1267 |
| | 11909 | | | | | |
| OSL216 | 11984- | 11984 | CCCCTAAACCTTGTACAGAATAT | CDS | 21 | 1268 |
| | 12006 | | | | | |
| OSL217 | 11985- | 11985 | CCCTAAACCTTGTACAGAATATG | CDS | 21 | 1269 |
| | 12007 | | | | | |
| OSL218 | 11990- | 11990 | AACCTTGTACAGAATATGAATAT | CDS | 21 | 1270 |
| | 12012 | | | | | |
| OSL219 | 11991- | 11991 | ACCTTGTACAGAATATGAATATA | CDS | 21 | 1271 |
| | 12013 | | | | | |
| OSL220 | 11994- | 11994 | TTGTACAGAATATGAATATAAGT | CDS | 21 | 1272 |
| | 12016 | | | | | |
| OSL221 | 12083- | 12083 | CCGATGAACTGGGTTGCAATAAA | CDS | 21 | 1273 |
| | 12105 | | | | | |
| OSL222 | 12114- | 12114 | AAGAACATGTGCTGAAAATATAT | CDS | 21 | 1274 |
| | 12136 | | | | | |
| OSL223 | 12140- | 12140 | AGCAAAATTGTACCCAATTAAAT | CDS | 21 | 1275 |
| | 12162 | | | | | |
| OSL224 | 12193- | 12193 | GGGTTCGAAACCAATGTTTTTGA | CDS | 21 | 1276 |
| | 12215 | | | | | |
| OSL225 | 12197- | 12197 | TCGAAACCAATGTTTTTGACAGA | CDS | 21 | 1277 |
| | 12219 | | | | | |
| OSL226 | 12389- | 12389 | CTGACAATGTCCGAATTCGAAAA | CDS | 21 | 1278 |
| | 12411 | | | | | |
| OSL227 | 12397- | 12397 | GTCCGAATTCGAAAATATAATCT | CDS | 21 | 1279 |
| | 12419 | | | | | |
| OSL228 | 12399- | 12399 | CCGAATTCGAAAATATAATCTCT | CDS | 21 | 1280 |
| | 12421 | | | | | |
| OSL229 | 12435- | 12435 | CTCAGAGTATCTTCAAGATGAGG | CDS | 21 | 1281 |
| | 12457 | | | | | |
| OSL230 | 12443- | 12443 | ATCTTCAAGATGAGGAATATATC | CDS | 21 | 1282 |
| | 12465 | | | | | |
| OSL231 | 12537- | 12537 | GGGCTCTAGGTTTGGTGCTATCAAA | CDS | 25 | 1283 |
| | 12561 | | | | | |
| OSL232 | 12564- | 12564 | TGCCTACATCCCCAACTTTGAAT | CDS | 21 | 1284 |
| | 12586 | | | | | |
| OSL233 | 12608- | 12608 | AGGAAGTTGACCTGAAACTGAAA | CDS | 21 | 1285 |
| | 12630 | | | | | |
| OSL234 | 12616- | 12616 | GACCTGAAACTGAAATACGTAAT | CDS | 21 | 1286 |
| | 12638 | | | | | |
| OSL235 | 12617- | 12617 | ACCTGAAACTGAAATACGTAATG | CDS | 21 | 1287 |
| | 12639 | | | | | |
| OSL236 | 12705 | 12705 | ACGCATTGAGGTGGCTAAACTTG | CDS | 21 | 1288 |
| | 12727 | | | | | |
| OSL237 | 12792- | 12792 | TCCCAAACTAGGGCTTATGTTCT | CDS | 21 | 1289 |
| | 12814 | | | | | |
| OSL238 | 12825- | 12825 | GGGAAAGGAACCTAAAATCGAGT | CDS | 21 | 1290 |
| | 12847 | | | | | |
| OSL239 | 12870- | 12870 | CCGCAACATCCTGGTTTTCGAGG | CDS | 21 | 1291 |
| | 12892 | | | | | |
| OSL240 | 12911- | 12911 | GCCTTTCTATCGATTATTTGAAC | CDS | 21 | 1292 |
| | 12933 | | | | | |
| OSL241 | 12915- | 12915 | TTCTATCGATTATTTGAACAATG | CDS | 21 | 1293 |
| | 12937 | | | | | |
| OSL242 | 12965- | 12965 | AGGACGTTATTGAAACCATAAAA | CDS | 21 | 1294 |
| | 12987 | | | | | |
| OSL243 | 12967 | 12967 | GACGTTATTGAAACCATAAAATA | CDS | 21 | 1295 |
| | 12989 | | | | | |
| OSL244 | 12968- | 12968 | ACGTTATTGAAACCATAAAATAT | CDS | 21 | 1296 |
| | 12990 | | | | | |
| OSL245 | 12995- | 12995 | GGACTGATAGGAGAGTCATTGCAAA | CDS | 21 | 1297 |
| | 13019 | | | | | |
| OSL246 | 13058- | 13058 | ACCAGTTATACTGGATATCTAAG | CDS | 25 | 1298 |
| | 13080 | | | | | |
| OSL247 | 13086- | 13086 | GGGAGAAGTATGGAAACAAAATA | CDS | 21 | 1299 |
| | 13108 | | | | | |
| OSL248 | 13091- | 13091 | AAGTATGGAAACAAAATAAATTT | CDS | 21 | 1300 |
| | 13113 | | | | | |
| OSL249 | 13175- | 13175 | TTCATCAACTCAGATACAATAAG | CDS | 21 | 1301 |
| | 13197 | | | | | |
| OSL250 | 13368- | 13368 | CGGAGGAAATTGCTATTTTGATG | CDS | 21 | 1302 |
| | 13390 | | | | | |
| OSL251 | 13371- | 13371 | AGGAAATTGCTATTTTGATGAGA | CDS | 21 | 1303 |
| | 13393 | | | | | |
| OSL252 | 13428- | 13428 | CACCGGAAAATATTGTGAAATGG | CDS | 21 | 1304 |
| | 13450 | | | | | |
| OSL253 | 13440- | 13440 | TTGTGAAATGGCGTTTTCAAAAG | CDS | 21 | 1305 |
| | 13462 | | | | | |
| OSL254 | 13541- | 13541 | CAGGATTCTTCCACTATAGAAGG | CDS | 21 | 1306 |
| | 13563 | | | | | |
| OSL255 | 13659- | 13659 | GGCAGATCTTAACATGGATATTG | CDS | 21 | 1307 |
| | 13681 | | | | | |
| OSL256 | 13725- | 13725 | GGCAATGAGTGAAGACTTTGTCA | CDS | 21 | 1308 |
| | 13747 | | | | | |
| OSL257 | 13842- | 13842 | ATCTGAAAATGTGGATAATAAGA | CDS | 21 | 1309 |
| | 13864 | | | | | |
| OSL258 | 13844- | 13844 | CTGAAAATGTGGATAATAAGAAT | CDS | 21 | 1310 |
| | 13866 | | | | | |
| OSL259 | 13852- | 13852 | GTGGATAATAAGAATTATGGAAG | CDS | 21 | 1311 |
| | 13874 | | | | | |
| OSL260 | 13853- | 13853 | TGGATAATAAGAATTATGGAAGT | CDS | 21 | 1312 |
| | 13875 | | | | | |
| OSL261 | 13951- | 13951 | TGGAATCTCTTCAAACGAAAATC | CDS | 21 | 1313 |
| | 13973 | | | | | |
| OSL262 | 13955- | 13955 | ATCTCTTCAAACGAAAATCTAAA | CDS | 21 | 1314 |
| | 13977 | | | | | |
| OSL263 | 13957- | 13957 | CTCTTCAAACGAAAATCTAAACA | CDS | 21 | 1315 |
| | 13979 | | | | | |
| OSL264 | 13960- | 13960 | TTCAAACGAAAATCTAAACAAAC | CDS | 21 | 1316 |
| | 13982 | | | | | |
| OSL265 | 13964- | 13964 | AACGAAAATCTAAACAAACTACC | CDS | 21 | 1317 |
| | 13986 | | | | | |
| OSL266 | 13976- | 13976 | AACAAACTACCAACTTTGAAAAT | CDS | 21 | 1318 |
| | 13998 | | | | | |
| OSL267 | 13983- | 13983 | TACCAACTTTGAAAATCCAATCT | CDS | 21 | 1319 |
| | 14005 | | | | | |
| OSL268 | 13987- | 13987 | AACTTTGAAAATCCAATCTATGC | CDS | 21 | 1320 |
| | 14009 | | | | | |
| OSL269 | 14121- | 14121 | TGCAACAGAAGACACTTTTAAAG | CDS | 21 | 1321 |
| | 14143 | | | | | |
| OSL270 | 14145- | 14145 | CACCGCAAATCTTGTTAAAGAAG | CDS | 21 | 1322 |
| | 14167 | | | | | |
| OSL271 | 14148- | 14148 | CGCAAATCTTGTTAAAGAAGACT | CDS | 21 | 1323 |
| | 14170 | | | | | |
| OSL272 | 14169- | 14169 | CTCTGAAGTATAGCTATACCAGC | CDS/3′- | 21 | 1324 |
| | 14191 | | | UTR | | |
| OSL273 | 14186- | 14186 | ACCAGCTATTTAGGGAATAATTA | 3′-UTR | 21 | 1325 |
| | 14208 | | | | | |
| OSL274 | 14188- | 14188 | CAGCTATTTAGGGAATAATTAGA | 3′-UTR | 21 | 1326 |
| | 14210 | | | | | |
| OSL275 | 14211- | 14211 | AACACACTTTTGCACATATATTT | 3′-UTR | 21 | 1327 |
| | 14233 | | | | | |
| OSL276 | 14213- | 14213 | CACACTTTTGCACATATATTTTT | 3′-UTR | 21 | 1328 |
| | 14235 | | | | | |
| OSL277 | 14236- | 14236 | TACAAACAGATGAAAAAAGTTAA | 3′-UTR | 21 | 1329 |
| | 14258 | | | | | |
| OSL278 | 14252- | 14252 | AAGTTAACATTCAGTACTTTATG | 3′-UTR | 21 | 1330 |
| | 14274 | | | | | |
| OSL279 | 14261- | 14261 | TTCAGTACTTTATGAAAAAAATA | 3′-UTR | 21 | 1331 |
| | 14283 | | | | | |
| OSL280 | 14263- | 14263 | CAGTACTTTATGAAAAAAATATA | 3′-UTR | 21 | 1332 |
| | 14285 | | | | | |
| OSL281 | 14341- | 14341 | CCGTCTCATATTTTTACAAATAA | 3′-UTR | 21 | 1333 |
| | 14363 | | | | | |
| OSL282 | 14355- | 14355 | TACAAATAATTATCACAATGTAC | 3′-UTR | 21 | 1334 |
| | 14377 | | | | | |
| OSL283 | 14366- | 14366 | ATCACAATGTACTATATGTATAT | 3′-UTR | 21 | 1335 |
| | 14388 | | | | | |
| OSL284 | 14368- | 14368 | CACAATGTACTATATGTATATCT | 3′-UTR | 21 | 1336 |
| | 14390 | | | | | |
| OSL285 | 14372- | 14372 | ATGTACTATATGTATATCTTTGC | 3′-UTR | 21 | 1337 |
| | 14394 | | | | | |
| OSL286 | 14396- | 14396 | CTGAAGTTGTCTGAAGGTAATAC | 3′-UTR | 21 | 1338 |
| | 14418 | | | | | |
| OSL287 | 14406- | 14406 | CTGAAGGTAATACTATAAATATA | 3′-UTR | 21 | 1339 |
| | 14428 | | | | | |
| OSL288 | 14437- | 14437 | TTGTAAATTTTGGAAAGATTATC | 3′-UTR | 21 | 1340 |
| | 14459 | | | | | |
| OSL289 | 14447- | 14447 | TGGAAAGATTATCCTGTTACTGA | 3′-UTR | 21 | 1341 |
| | 14469 | | | | | |
| OSL290 | 14451- | 14451 | AAGATTATCCTGTTACTGAATTT | 3′-UTR | 21 | 1342 |
| | 14473 | | | | | |
| OSL291 | 14457- | 14457 | ATCCTGTTACTGAATTTGCTAAT | 3′-UTR | 21 | 1343 |
| | 14479 | | | | | |
| OSL292 | 14458- | 14458 | TCCTGTTACTGAATTTGCTAATA | 3′-UTR | 21 | 1344 |
| | 14480 | | | | | |
| OSL293 | 14464- | 14464 | TACTGAATTTGCTAATAAAGATG | 3′-UTR | 21 | 1345 |
| | 14486 | | | | | |
| OSL294 | 14503- | 14503 | GTGATCATTATAGTAAATGATCC | 3′-UTR | 21 | 1346 |
| | 14525 | | | | | |
| OSL295 | 14513- | 14513 | TAGTAAATGATCCAACAAGAAAA | 3′-UTR | 21 | 1347 |
| | 14535 | | | | | |
| OSL296 | 14523- | 14523 | TCCAACAAGAAAAGGAATTGACT | 3′-UTR | 21 | 1348 |
| | 14545 | | | | | |
| OSL297 | 14579- | 14579 | CACTCATATTTCCTATAAAATTA | 3′-UTR | 21 | 1349 |
| | 14601 | | | | | |
| OSL298 | 14581. | 14581 | CTCATATTTCCTATAAAATTATC | 3′-UTR | 21 | 1350 |
| | 14603 | | | | | |
| OSL299 | 14633- | 14633 | GTCCATTTTTACACATTAGCACT | 3′-UTR | 21 | 1351 |
| | 14655 | | | | | |
| OSL300 | 14649- | 14649 | TAGCACTTAATTAATGTTCAATA | 3′-UTR | 21 | 1352 |
| | 14671 | | | | | |
| OSL301 | 14650- | 14650 | AGCACTTAATTAATGTTCAATAT | 3′-UTR | 21 | 1353 |
| | 14672 | | | | | |
| OSL302 | 14652- | 14652 | CACTTAATTAATGTTCAATATTA | 3′-UTR | 21 | 1354 |
| | 14674 | | | | | |
| OSL303 | 14665- | 14665 | TTCAATATTACATGTCAATTTGA | 3′-UTR | 21 | 1355 |
| | 14687 | | | | | |
| OSL304 | 14684- | 14684 | TTGATTAATGGCTATGTTGATAG | 3′-UTR | 21 | 1356 |
| | 14706 | | | | | |
| OSL305 | 14708- | 14708 | GGCCACTATGTGTTGTATAGACA | 3′-UTR | 21 | 1357 |
| | 14730 | | | | | |
| OSL306 | 14711- | 14711 | CACTATGTGTTGTATAGACATCT | 3′-UTR | 21 | 1358 |
| | 14733 | | | | | |
| OSL307 | 14767- | 14767 | AGGTAGGAAAAGCAATTCAGTTT | 3′-UTR | 21 | 1359 |
| | 14789 | | | | | |
| OSL308 | 14856- | 14856 | GCCAAGACAACATTTTTATTTGT | 3′-UTR | 21 | 1360 |
| | 14878 | | | | | |
| OSL309 | 14861- | 14861 | GACAACATTTTTATTTGTGATGT | 3′-UTR | 21 | 1361 |
| | 14883 | | | | | |
| OSL310 | 14886- | 14886 | ATGAGGAAATCCCATATCATTAA | 3′-UTR | 21 | 1362 |
| | 14908 | | | | | |
| OSL311 | 14921- | 14921 | TGCATTGAGTTTGTGGTTAATTA | 3′-UTR | 21 | 1363 |
| | 14943 | | | | | |
| OSL312 | 14925- | 14925 | TTGAGTTTGTGGTTAATTAAATG | 3′-UTR | 21 | 1364 |
| | 14947 | | | | | |
| OSL313 | 15034 | 15034 | AGCTGAATTTCTGAAACCAAATC | 3′-UTR | 21 | 1365 |
| | 15056 | | | | | |
| OSL314 | 15049- | 15049 | ACCAAATCTGTGTCTTCATAAAA | 3′-UTR | 21 | 1366 |
| | 15071 | | | | | |
| OSL315 | 15115- | 15115 | TGGGTTTTTGTTTCTATGAAAAT | 3′-UTR | 21 | 1367 |
| | 15137 | | | | | |
| OSL316 | 15122- | 15122 | TTGTTTCTATGAAAATATCATTA | 3′-UTR | 21 | 1368 |
| | 15144 | | | | | |
| OSL317 | 15126- | 15126 | TTCTATGAAAATATCATTATAAT | 3′-UTR | 21 | 1369 |
| | 15148 | | | | | |
| OSL318 | 15130- | 15130 | ATGAAAATATCATTATAATCACT | 3′-UTR | 21 | 1370 |
| | 15152 | | | | | |
| OSL319 | 15138- | 15138 | ATCATTATAATCACTATTTATTT | 3′-UTR | 21 | 1371 |
| | 15160 | | | | | |
| OSL320 | 15188- | 15188 | AACCATTCTTATTAAGCTTTTTA | 3′-UTR | 21 | 1372 |
| | 15210 | | | | | |
| OSL321 | 15189- | 15189 | ACCATTCTTATTAAGCTTTTTAT | 3′-UTR | 21 | 1373 |
| | 15211 | | | | | |
| OSL322 | 15220- | 15220 | GTGGCTAAATGTGTACATTTATA | 3′-UTR | 21 | 1374 |
| | 15242 | | | | | |
| OSL323 | 15221- | 15221 | TGGCTAAATGTGTACATTTATAT | 3′-UTR | 21 | 1375 |
| | 15243 | | | | | |
| OSL324 | 15222- | 15222 | GGCTAAATGTGTACATTTATATT | 3′-UTR | 21 | 1376 |
| | 15244 | | | | | |
| OSL325 | 15228- | 15228 | ATGTGTACATTTATATTAGAATG | 3′-UTR | 21 | 1377 |
| | 15250 | | | | | |
| OSL326 | 15263- | 15263 | CAGATCTTTTCTTTAATTCTTAT | 3′-UTR | 21 | 1378 |
| | 15285 | | | | | |
| OSL327 | 15266- | 15266 | ATCTTTTCTTTAATTCTTATTGG | 3′-UTR | 21 | 1379 |
| | 15288 | | | | | |
| OSL328 | 15271- | 15271 | TTCTTTAATTCTTATTGGTTTTT | 3′-UTR | 21 | 1380 |
| | 15293 | | | | | |
| OSL329 | 15438- | 15438 | CTCTTAAATAGTGGGTATAGTCT | 3′-UTR | 21 | 1381 |
| | 15460 | | | | | |
| OSL330 | 15524- | 15524 | GTGCTAATATTGCACATTTGTTA | 3′-UTR | 21 | 1382 |
| | 15546 | | | | | |
| OSL331 | 15525- | 15525 | TGCTAATATTGCACATTTGTTAA | 3′-UTR | 21 | 1383 |
| | 15547 | | | | | |
| OSL332 | 15575- | 15575 | ATGGATGAATGAATGAAACATAT | 3′-UTR | 21 | 1384 |
| | 15597 | | | | | |
| OSL333 | 15583- | 15583 | ATGAATGAAACATATACTACTGA | 3′-UTR | 21 | 1385 |
| | 15605 | | | | | |
| OSL334 | 15587- | 15587 | ATGAAACATATACTACTGATTAT | 3′-UTR | 21 | 1386 |
| | 15609 | | | | | |
| OSL335 | 15591- | 15591 | AACATATACTACTGATTATTTTA | 3′-UTR | 21 | 1387 |
| | 15613 | | | | | |
| OSL336 | 15655- | 15655 | CTGACTGTAATTACTTTGATTAG | 3′-UTR | 21 | 1388 |
| | 15677 | | | | | |
| OSL337 | 15659- | 15659 | CTGTAATTACTTTGATTAGATAA | 3′-UTR | 21 | 1389 |
| | 15681 | | | | | |
| OSL338 | 15675- | 15675 | TAGATAAACAACTGGAAATAATG | 3′-UTR | 21 | 1390 |
| | 15697 | | | | | |
| OSL339 | 15698- | 15698 | CTGCTGAAAAAGTTCTAATAAAT | 3′-UTR | 21 | 1391 |
| | 15720 | | | | | |
| OSL340 | 15699- | 15699 | TGCTGAAAAAGTTCTAATAAATG | 3′-UTR | 21 | 1392 |
| | 15721 | | | | | |

TABLE 11
Additional table of siRNA sequences

| SEQ | | antisense sequence | | sense sequence | SEQ ID |
|---|---|---|---|---|---|
| ID | OSID | (5′ to 3′) | OSID | (3′ to 5′) | NO:S |
| 957 | OSC17C-1 | CAGUUGCGCAGUUUCUUGUC | OSC17B-1 | [dT][dT]GUCAACGCGUCA | 973 |
| | | AGUUC[dT][dT] | | AAGAACAGUCAAG | |
| 958 | OSC17C-2 | CAGUUGCGCAGUUUCUUGU | OSC17B-2 | [dT][dT]*G*UCAACGCGUC | 974 |
| | | [mC][mA]GUUC[dT][dT] | | AAAGAACAGUCAAG | |
| 959 | OSC17C-3 | CAGUUGCGCAGUUUCU[mU] | OSC17B-3 | [dT][dT]*G*UCAACGCGUC | 975 |
| | | [mG]UCAGUUC[dT][dT] | | AAAGAACAG[mU]CAAG | |
| 960 | OSC17C-4 | CAGUUGCGCAGUUUCU[mU] | OSC17B-4 | [dT][dT]*G*UCAACGCGUC | 976 |
| | | [mG]U[mC][mA]GUUC[dT] | | AAAGAA[mC]AGUCAAG | |
| | | [dT] | | | |
| | | | OSC17B-5 | [dT][dT]*G*UCAACGCGUC | 977 |
| | | | | AAAGAA[mC]AG[mU]CAA | |
| | | | | G | |
| | | | OSC17B-6 | [dT][dT]*G*UCAA[mC]GCG | 978 |
| | | | | UCAAAGAA[mC]AG[mU]C | |
| | | | | AAG | |
| 961 | OSC47C-1 | AAGAGCUCAGGUCUCUGAGG | OSC47B-1 | [dT][dT]UUCUCGAGUCCA | 979 |
| | | G[dT][dT] | | GAGACUCCC | |
| 962 | OSC47C-2 | AAGAGCUCAGGUCUC[mU]GA | OSC47B-2 | [dT][dT]*U*UCUCGAGUCC | 980 |
| | | GGG[dT][dT] | | AGAGACUCCC | |
| 963 | OSC47C-3 | AAGAGCUCAGGUCUC[mU][mG] | OSC47B-3 | [dT][dT]*U*UCUCGAGUCC | 981 |
| | | AGGG[dT][dT] | | AGAGA[mC]UCCC | |
| | | | OSC47B-4 | [dT][dT]*U*UCUCGAGUCC | 982 |
| | | | | AGAG[mA][mC]UCCC | |
| | | | OSC47B-5 | [dT][dT]*U*UCUCGAG[mU] | 983 |
| | | | | CCAGAG[mA][mC]UCCC | |
| 964 | OSL231C-1 | UUUGAUAGCACCAAACCUAGA | OSL231B-1 | [dT][dT]AAACUAUCGUGG | 984 |
| | | GCCC[dT][dT] | | UUUGGAUCUCGGG | |
| 965 | OSL231C-2 | UUUGA[mU]AG[mC]ACCAAACC | OSL231B-2 | [dT][dT]*A*AACUAUCGUG | 985 |
| | | [mU]AGAGCCC[dT][dT] | | GUUUGGAUCUCGGG | |
| 966 | OSL231C-3 | UUUGAUAGCACCAAACC[mU] | OSL231B-3 | [dT][dT]*A*AACUA[mU]CG | 986 |
| | | [mA]GAGCCC[dT][dT] | | [mU]GGUUUGGA[mU]CUC | |
| | | | | GGG | |
| 967 | OSL231C-4 | UUUGAUAG[mC]ACCAAACC | OSL231B-4 | [dT][dT]*A*AACU[mA][mU] | 987 |
| | | [mU]AGAGCCC[dT][dT] | | C[mG][mU]GGUUUGG[mA] | |
| | | | | [mU]CUCGGG | |
| 968 | OSL231C-5 | UUUGAUAGCACCAAACC[mU]A | OSL231B-5 | [dT][dT]*A*AACUGUCGUG | 988 |
| | | GAGCCC[dT][dT] | | GUUUGGA[mU]CUCGGG | |
| | | | OSL231B-6 | [dT][dT]*A*AACUGUCG | 989 |
| | | | | [mU]GGUUUGGA[mU]CUCG | |
| | | | | GG | |
| | | | OSL231B-7 | [dT][dT]*A*AACUAUCGUG | 990 |
| | | | | G[mU]UUGGAUCUCGGG | |
| 969 | OSL245C-1 | UUUGCAAUGACUCUCCUAUCA | OSL245B-1 | [dT][dT]AAACGUUACUGA | 991 |
| | | GUCC[dT][dT] | | GAGGAUAGUCAGG | |
| 970 | OSL245C-2 | UUUGCAA[mU][mG]ACUCUCC | OSL245B-2 | [dT][dT]*A*AACG[mU]UA | 992 |
| | | [mU][mA]UCAGUCC[dT][dT] | | [mC]UGAGAGGA[mU]AGUC | |
| | | | | AGG | |
| 971 | OSL245C-3 | UUUGCAAUGACUCUCC[mU] | OSL245B-3 | [dT][dT]*A*AACGUUACUG | 993 |
| | | [mA]UCAGUCC[dT][dT] | | AGAGGA[mU]AGUCAGG | |
| 972 | OSL245C-4 | UUUGCAA[mU][mG]ACUCUCCU | OSL245B-4 | [dT][dT]*A*AACGUUA[mC] | 994 |
| | | AUCAGUCC[dT][dT] | | UGAGAGGA[mU]AGUCAG | |
| | | | | G | |
| | | | OSL245B-5 | [dT][dT]*A*AACGUUACUG | 995 |
| | | | | AGAGGAUAGUCAGG | |

Key to modifications
[dT] = DNA base (T) within RNA oligo
[mA], [mG], [mC], [mU] = 2′O-Methyl RNA
* = Phosphorothioate linkages

In one embodiment, an RNAi (e.g., a dsRNA) featured herein includes a first sequence of a dsRNA that is selected from the group including the sense sequences of any table herein and a second sequence that is selected from the group consisting of the corresponding antisense sequences of any table herein. A corresponding antisense sequence is a nucleotide sequence within the OSID family for example OSC17. In those instances when we refer to an siRNA with no suffix (e.g., OSC17), we mean that to indicate the dsRNA comprised of the antisense and sense strands corresponding to that number (e.g., OSC17A paired with OSC17S or OSC17C-(n) paired with OSC17B-(n) where “n” is any number of the OSC17 family).

Unless otherwise specified, the compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation. It is understood that the phosphorothioate group, designated by an asterisk (*), constitutes a stereogenic center, and the presence of each such group in a sequence engenders two diastereoisomers. The number of such diastereoisomers in a double stranded RNAi agent may be calculated by the formula 2\hat{ }n, wherein n represents the number of phosphorothioate groups in a sequence comprised of a double stranded siRNA.

In some embodiments, the antisense strand (identified with “A” in the OS ID name) and/or the sense strand (identified with “S” in the OS ID name) of an RNAi agent comprises or consists of a nucleobase sequence, for example, “OSC17A-1” CAGUUGCGCAGUUUCUUGUCAGUUC[dT][dT] (SEQ ID NO: 17), and the nucleobase sequence may include at least one or more nucleotides as a modified nucleotide, and wherein SEQ ID NO: 17 is located at positions 1 to 25 (5′→3′) of the antisense strand and forms a duplex with the corresponding sense strand (identified as OSC17S-1. In some embodiments, the antisense strand of an RNAi agent comprises or consists of a nucleobase sequence for example CAGUUGCGCAGUUUCUUGUCAGUUC[dT][dT] (SEQ ID NO: 17), wherein all or substantially all or 1, 2, 3, 4 or 5 of the nucleotides are modified nucleotides (see for example SEQ ID NO. 24), and wherein SEQ ID NO: 24 is located at positions 1 to 27 (5′→3′) of the antisense strand. For any antisense or sense strand disclosed herein, in some embodiments, the antisense strand of an RNAi agent comprises or consists of the sequence (5′→3′) wherein * is a phosphorothioate linkage between deoxy thymine [dT]; and/or wherein mC, mA, mG, mU are 2′-O-methyl cytidine, 2′-O-methyl adenine, 2′-O-methyl guanosine, 2′-O-methyl uridine respectively; and/or wherein 2fA, 2fU, 2fG, 2fC are 2′-fluoro adenine, 2′-fluoro uridine, 2′-fluoro guanosine, and 2′-fluoro cytosine respectively. The antisense target on the mRNA is identified with the same name but without the notation of “A” or “S” after the name. An antisense sequence with the same name, for example OSC17A-1 through OSC17A-18 binds to the same nucleotide target sequence.

Sequences shown in Table 4 were transfected into HEK 293 (human embryonic kidney) and MDA-MB-435S (human melanoma) cell lines to determine their ability to reduce the protein expression of LRP2 and CD320 gene/protein. These two cell lines were chosen because of their relatively high expression levels of LRP2 as noted in the Human Protein Atlas at world wide web.proteinatlas.org and the NCI-60 gene expression profiles at discover.nci.nih.gov/cellminer/so that a change in protein expression for LRP2 was easy to detect.

Referring now to FIGS. 3A-B and FIG. 3D-E, HEK293 and MDA-MB-231 cells were transfected with 20 nM of indicated siRNAs and incubated for 48 hours. Whole cell lysates were prepared and immunoblotted for CD320 and LRP2 protein levels. The protein levels were normalized to a housekeeping control gene unaffected by the siRNA transfection. The graphs represent the fold change of protein levels compared to the scrambled siRNA control (OSS1). (Average−/+SEM is shown, n=3).

CD320 and LRP2 protein levels were determined by western blot and quantified by Image Studio Software (LiCor Company), relative to a control protein that is not affected by CD320 or LRP2 knockdown. To determine the efficacy of knockdown, protein levels of CD320 (FIGS. 3A-B) and LRP2 (FIGS. 3 D-E) on the samples that were exposed to siRNA sequences against the mRNA of either gene, were compared to that in the untreated and scrambled controls (black and gray bars, respectively, in all graphs of FIG. 3). We found that both siRNA sequences directed against CD320 (OSC17 and OSC47) almost completely abrogated CD320 expression (circles in FIG. 3A-B). siLRP2 sequences resulted in variable efficiency in reducing LRP2 protein. Two sequences (OSL231 and OSL245) consistently reduced LRP2 levels 75% or more in both cell lines (circles FIGS. 3 B, E).

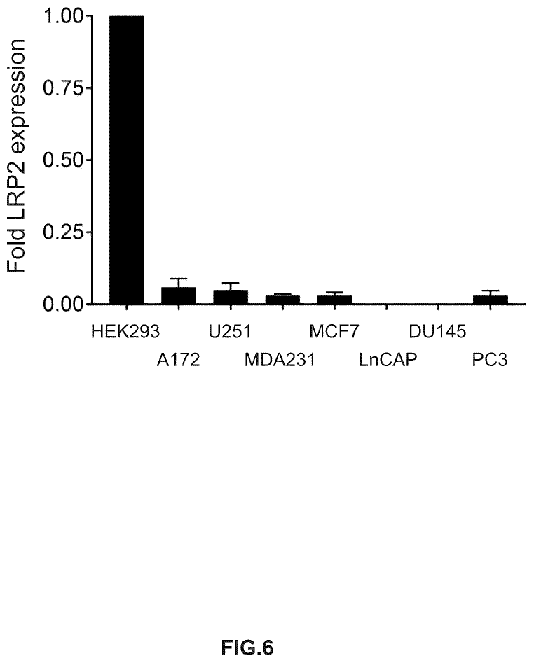

Referring now to FIG. 6, lysates were made from transformed (HEK293) and representative cancer cell lines, and western blot was performed to determine LRP2 protein levels. The cancer cells screened have low levels of LRP2 expression. The results represent the averages+SEM of three independent lysates. The data suggests that the cancer cells screened have very low levels of LRP2 expression.

We transfected a panel of LRP2 and CD320 siRNAs into cancer cell lines derived from multiple tissues and analyzed the levels of LRP2 protein and CD320 protein in the cell line. Representative cell lines from prostate, breast and glioblastoma, and normal fibroblasts were exposed to CD320 and LRP2 siRNAs in an experimental set-up similar to that described for HEK293 and MDA-MB-435S cells. The results are shown in FIGS. 3 C, F, and FIG. 4.

Referring now to FIGS. 3 C, F; and FIG. 4, MDA-MB-231 LnCAP, MCF-7 and U251 cells were exposed to siRNA sequences to knockdown CD320 (FIG. 3C, and FIGS. 4A-C) and LRP2 (FIG. 3 F, and FIGS. 4 D-F), in a similar fashion as described for the data represented in FIGS. 3A, B, D, and E. CD320 protein knockdown FIG. 3C, and FIG. 4A-C, compared to the untreated or scrambled controls, is more than 90% for all cell lines tested. LRP2 knockdown is accomplished in all cell lines too. However, the level of knockdown is less in the LnCAP cells compared to the other cell lines and the sequences that are effective may differ as well (FIG. 3 F, and FIGS. 4 D-F).

Referring now to FIG. 5, an experimental set up similar to that described in FIG. 3 was employed. Additional prostate and brain cancer cell lines, as well as normal fibroblast, were exposed to siRNAs directed against CD320. Levels of CD320 were nearly abrogated in DU-145 (prostate) cells, whereas the levels of knockdown in A172 brain cells and normal fibroblasts were 21%-33% and 25%-28%, respectively (FIG. 5A-C).

From these studies we can conclude that two siRNAs to CD320 (OSC17 and OSC47) are very effective in knocking down CD320 protein levels (80% or more), in nearly every cell line tested. While LRP2 is theoretically harder to knock down because of its size, we have identified two siRNAs, OSL231 and OSL245, that consistently knock down LRP2 in most cell lines in which we can detect LRP2.

In addition, LRP2 protein expression levels are very high in HEK 293 cells and easily detectable by western blot. Cancer cell lines have much lower expression of LRP2 compared to HEK293 cells as measured by western blot (FIG. 6), and some cell lines may contain LRP2 at levels below reliable detection.

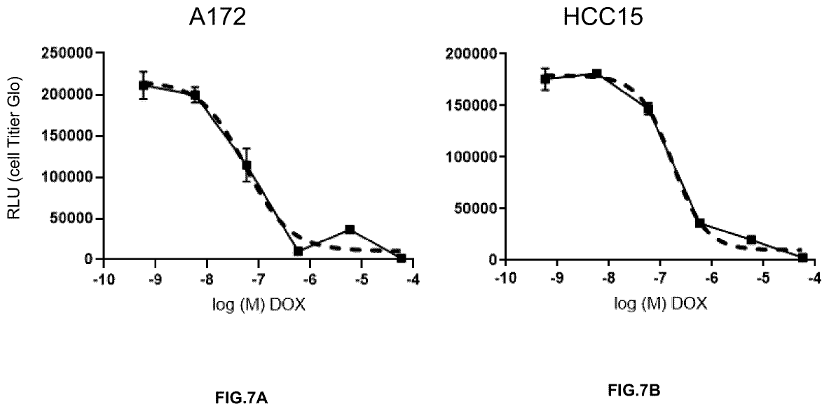

Referring now to FIG. 7, the effects of doxorubicin treatment on cell viability, as measured by the CTG assay, are illustrated. A172 and HCC15 cells were plated at 1200 cells/well in a 96 well plate. The next day, cells were treated with doxorubicin at the indicated concentrations. Four days after the doxorubicin exposure was initiated, the cells were assayed for viability using the CTG assay. The line indicates the non-linear fitting of the data to calculate an IC₅₀ value. Instead of visually assessing the effect of CD320/LRP2 gene expression knockdown on cell proliferation (as shown for shRNA-mediated CD320/LRP2 knockdown in FIG. 2), a functional assay for quantitating the effect on cell viability of the simultaneous knockdown of LRP2 and CD320 by siRNA was developed. A widely used assay for the measurement of cell viability is the Promega Cell-titer GLO® platform (CTG), which quantifies ATP levels in the cell (live cells produce ATP, dead cells do not). After incubating the cells with the CTG reagent, ATP levels can be indirectly measured as light production using the TECAN luminescence plate reader. As a first step, toxicity of a known chemotherapeutic drug, doxorubicin, was assayed on the cell lines of interest. Doxorubicin was used as a positive control for cell toxicity in our assay. Representative data from A172 brain cancer cells (FIG. 7A) and HCC15 lung cancer cells (FIG. 7B) exposed to doxorubicin are shown in FIG. 7. From this data, the IC₅₀ of doxorubicin treatment on these cell lines was determined: 132 nm for A172 cells and 167 nm for HCC15 cells. Based upon these findings, a larger screen was initiated to determine the IC₅₀ of doxorubicin in several cancer and non-cancer cell lines, to determine the doxorubicin dose to use when cell lines are used in the viability assay to test the simultaneous knockdown of CD320 and LRP2. The results of the cell lines tested are summarized in Table 8 IC₅₀ determination of doxorubicin.

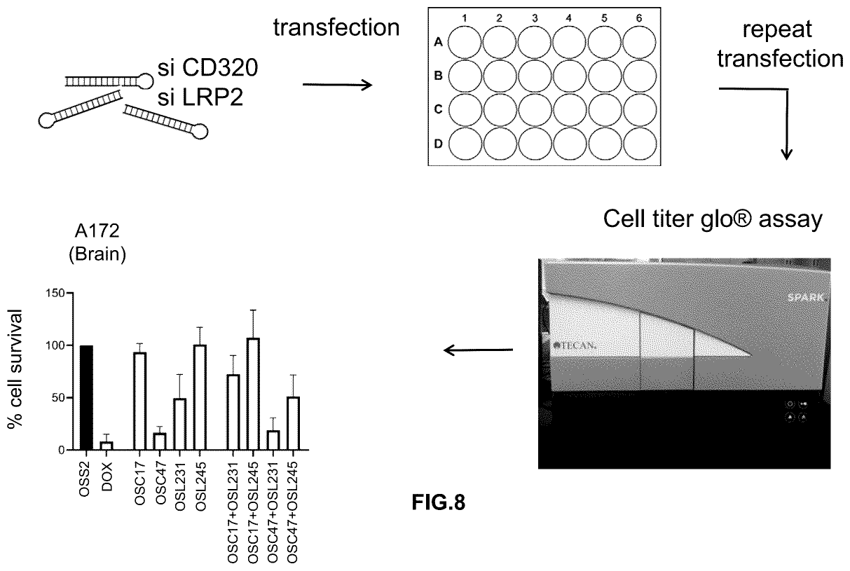

To quantify the effects of knocking down CD320 and LRP2 on cell proliferation, cells are plated in a 24-well plate. The next day, the cells are transfected with siRNAs to CD320 and/or LRP2. The cell lines may require repeated transfections and/or time for efficient toxicity (cell line dependent). In this experimental set-up there is room for repeat infection should some cell lines require that for efficient toxicity. At the end of the study, the cell lines are analyzed for cell growth by the CTG assay. A schematic of this experimental setup is presented in FIG. 8.

TABLE 8

| Cancer type | Cell line | IC₅₀ (nM) |
|---|---|---|
| Glioblastoma | A172 | 132 |
| | U251 | 24 |
| Breast | MDA-MB-231 | 43 |
| | MCF7 | 121 |
| Prostate | DU145 | 248 |
| | PC3 | 387 |
| Lung | NCI-H460 | 56 |
| | A549 | 126 |
| | HCC15 | 167 |
| Melanoma | MDA-MB-435S | 325 |
| Other | GM05659 | 267 |
| | HEK293 | 29 |

The cells lines were plated at 1,000 to 4,000 cells/well in a 96-well plate and treated with doxorubicin the following day. CTG activity was measured 4 days after treatment. IC₅₀ values were calculated by GraphPad Prism Software. Results are tabulated in Table 8.

These data show that doxorubicin works efficiently on this CTG platform (i.e., doxorubicin kills cancer cells) and can thus be used as a positive control in the in vitro assay to compare the cytotoxic effects of siRNA-knockdown of CD320 and LRP2. In this latter assay, normal or cancer cells are transfected with individual or combinations of siRNAs sequences that are targeting CD320 or LRP2 specifically or control siRNAs, similar to the experiments that provided the data for FIGS. 3, 4, and 5. In FIGS. 3, 4, and 5, protein levels are measured, but in the in vitro assay, cell viability is measured.

Referring now to FIG. 8, an overview of a functional assay for screening (ds) siRNA effects on cell proliferation is illustrated. To quantify the effects of knocking down CD320 and LRP2 on cell proliferation, cells are plated in a 24-well plate. The next day, the cells are transfected with siRNAs to CD320 and/or LRP2. The cell lines may require repeated transfections and/or time for efficient toxicity (cell line dependent). In this experimental set-up there is room for repeat infection should some cell lines require that for efficient toxicity. At the end of the study, the cell lines were analyzed for cell growth by the CTG assay.

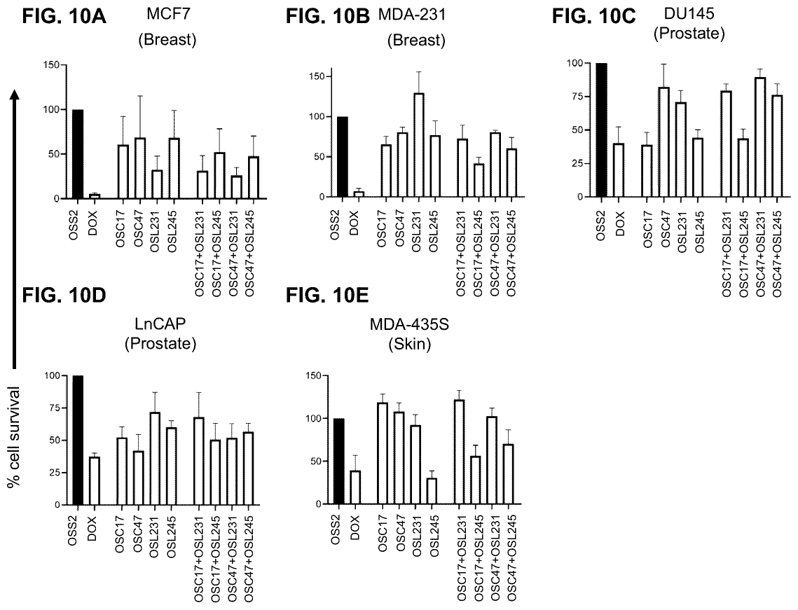

Now, referring to FIG. 10B, MDA-MB-231 triple negative breast cancer cells were plated in a 24-well plate at 20,000 cells/well. Cells were transfected the next day with an siRNA selected from the group of OSC17, OSC47, OSL231, and OSL245 at 20 nM. Cells were also transfected with combinations of two siRNAs each of 10 nM, one of these targeting CD320 and the other LRP2, with the siRNAs targeting CD320 selected from the group of OSC17 and OSC47, and the LRP2 targeting siRNAs selected from the group of OSL231 and OSL245, each dosed at 10 nM. Cells were repeated transfected 4 times over the course of 11 days as indicated in Table 9. At day 11, cells were analyzed for cell growth by the CTG assay. The percent cell survival compared to the non-targeting control (OSS2) is shown. The data represented is the average of 6 experiments−/+SEM.

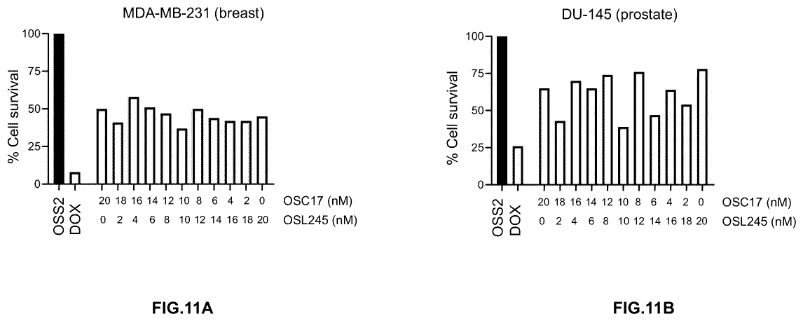

Now, referring to FIG. 11, MDA-MB-231 and DU-145 cells were transfected with 20 nM of the negative control siRNA (OSS2), 20 nM siRNA targeting CD320 (OSC17), or 20 nM siRNA targeting LRP2 (OSL245). Cells were also transfected with a combination of a CD320 targeting siRNA (OSC17) and LRP2 targeting siRNA (OSL24), over a range of concentrations (2-20 nM), so the concentration of the two siRNAs equaled 20 nM total siRNA transfected, as indicated in FIG. 11. Cells were repeatedly transfected as indicated in Table 9, and the percent cell survival is shown.

Now, referring to FIG. 12, MDA-MB-231 breast cancer cells were transfected with 20 nM of the negative control siRNA (OSS2), 20 nM siRNA targeting CD320 (OSC17), or 20 nM siRNA targeting LRP2 (OSL245). Each day, over five days, lysates were prepared. Western blotting was performed on the lysates for CD320 protein levels (FIG. 12A) or LRP2 protein levels (FIG. 12B).

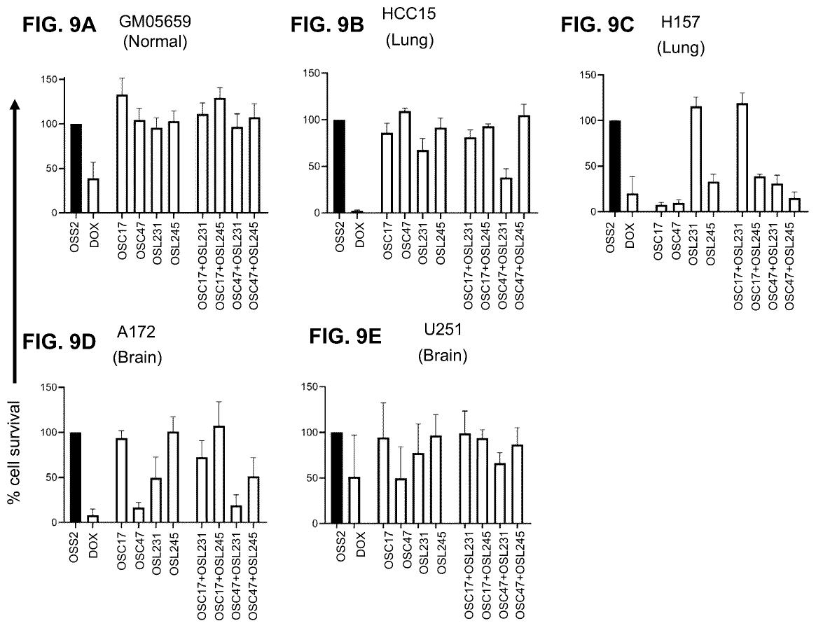

Referring now to FIG. 9 and FIG. 10, data quantifying the effects of knocking down CD320 and LRP2 in various cell lines is represented. Cell lines representative of several types of cancers or normal fibroblasts were transfected with individual or combinations of siRNAs to CD320 or LRP2 as indicated. Cells were repeatedly transfected as outlined in Table 9 for efficient toxicity, then assayed for viability by the CTG assay. Doxorubicin treated cells served as a positive control for cell toxicity in our assays.

The data of the individual experiments presented in FIG. 9 and FIG. 10 and additional cell lines we have screened are summarized in Table 9. These experiments show the broad applicability of siCD320 and siLRP2 toxicity in a variety of cancer types.

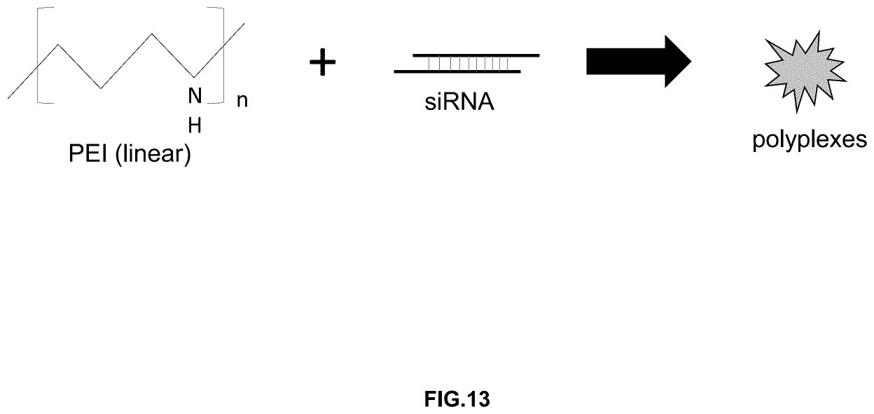

Referring now to FIG. 13, a schematic of PEI and siRNA complexes is illustrated. PEI and siRNAs are mixed together. Subsequently, polyplexes (a nanoparticle, broadly speaking) form of the PEI-siRNA complex, which are able to enter the cell via an endocytotic or pinocytotic mechanism.

Referring now to FIG. 14, siRNAs are short RNA duplexes of generally 16 to 30 nucleotides; the sequence of the siRNA is complementary to a mRNA expressed in the cell. Exogenous siRNA duplexes are introduced into the cell via a method of transfection. The siRNA duplexes are unwound via the RISC (RNA-induced silencing complex) complex, whereby the guide strand of the siRNA hybridizes with its complementary mRNA molecule. The mRNA is degraded by the RISC/AGO complex, which has RNAse cleave activity. The end result is that the mRNA targeted by the siRNA is degraded, and the protein encoded by the mRNA is not produced. This causes the “knockdown” effect or reduced protein levels of the gene targeted by the siRNA compared to control-treated cells.

Referring now to FIG. 15 effectiveness of INTERFERin in delivering siRNAs to cancer cells is illustrated. 2 nM of indicated siRNAs were transfected into A172 and MDA-MB-435S cells as per the manufacturers protocol. Cell lysates were prepared 3 days post-infection and analyzed by western blot for CD320 protein levels. OSS1 and OSS2 are non-targeting siRNA controls. In this experiment, both sequences were tested. CD320 protein levels were knocked down to 9% to 18% for A172 cells and 26% to 48% for MDA-MB-435S cells, compared to OSS1. Much more efficient knockdown of CD320 is observed when the siRNAs were delivered with other transfection reagents (e.g. RNAiMAX, Viromer Blue) that were used in the experiments described previously particularly for MDA-MB-435S cells. The Polyplus INTERFERin platform has been tested in vitro in our laboratory in a proof of principle experiment, whereby the platform is able to deliver siRNAs to the target cells in vitro.

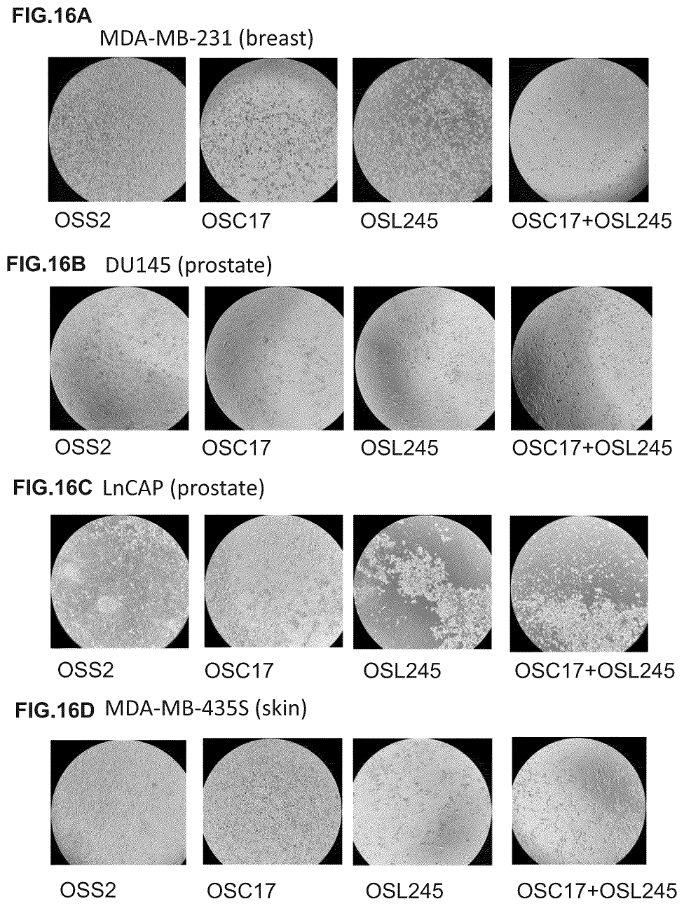

Referring now to FIG. 16, treatment of breast, prostate, and skin cancer cells with an inhibitor of CD320 receptor or an inhibitor of LRP2 receptor or a combination of both in an amount effective to inhibit proliferation of the cancer cells as compared to the control cells treated with control SiRNA is illustrated. MDA-MB-231, DU145, LnCAP, and MDA-MB-435S cells were plated at 20,000 cells per well in a 24-well plate. The next day, the cells were transected with 20 nM of indicated siRNAs to knock down CD320, LRP2, or scrambled control. For the combination of siRNAs, cells were treated with 10 nM of each siRNA for 20 nM total treatments. Cells were repeatedly transfected as in Table 9 for the length of time indicated in Table 9. The indicated pictures of the cells were taken at the end of the experiment.

TABLE 9
Summary of functional siRNA data screening

| | | | | Single siRNA | | | | Double siRNA | | | | |
|---|---|---|---|---|---|---|---|---|---|---|---|---|
| | | | | knockdown | | | | knockdown | | | | |
| | | | | (siCD320) | | (siLRP2) | | OSC17+ | | OSC47+ | | |
| | | # of | Days | OSC- | OSO- | OSL- | OSL- | OSL | OSL | OSL | OSL | |
| Cell line | n | txns | expt | 17 | 47 | 231 | 245 | 231 | 245 | 231 | 245 | DOX |
| Normal | | | | | | | | | | | | |
| GM05659 | 3 | 4 | 7 | 133 | 104 | 96 | 103 | 111 | 129 | 97 | 107 | 39 |
| Lung | | | | | | | | | | | | |
| HCC15 | 3 | 2 | 7 | 86 | 109 | 68 | 92 | 81 | 93 | 38 | 105 | 2 |
| H157 | 3 | 5 | 8 | 7 | 9 | 116 | 33 | 119 | 39 | 31 | 15 | 20 |
| Melanoma | | | | | | | | | | | | |
| MDA-MB-435S | 4 | 5 | 10 | 119 | 108 | 92 | 31 | 122 | 56 | 103 | 70 | 51 |
| Prostate | | | | | | | | | | | | |
| LnCAP | 3 | 4 | 7 | 52 | 42 | 72 | 60 | 68 | 51 | 52 | 57 | 38 |
| DU-145 | 6 | 4 | 7 | 39 | 82 | 71 | 44 | 80 | 44 | 90 | 76 | 40 |
| Glioblastoma | | | | | | | | | | | | |
| A172 | 3 | 5 | 7 | 94 | 17 | 50 | 101 | 73 | 107 | 19 | 51 | 8 |
| U251 | 4 | 6 | 8 | 94 | 50 | 77 | 97 | 99 | 94 | 66 | 87 | 51 |
| Breast | | | | | | | | | | | | |
| MCF-7 | 3 | 2 | 7 | 61 | 69 | 32 | 68 | 31 | 52 | 26 | 48 | 5 |
| MDA-MB-231 | 6 | 4 | 11 | 66 | 81 | 130 | 77 | 71 | 44 | 81 | 61 | 7 |

Note:
The numbers represent percent survival compared to negative control OSS2.

A murine human tumor xenograft model was established using triple-negative breast cancer cells (MDA-MB-231) injected into the flanks of nude mice to test the efficacy of combined dosing of OSC17 and OSL245. The administration of the drug is by repeated dosing over a range of drug concentrations using intratumoral, iv, ip or specialized route of administration. The dosing schedule is based on pilot studies to determine the tolerability of the delivery vehicle and the drug and will incorporate ranges that are taught in the art. Among the delivery platforms are nanoparticles, liposomes, micelles, polymers, small molecule conjugates, aptamers and antibody conjugates. Hybrid technologies containing elements of the aforementioned delivery systems are also known.

The manufacturing process consists of synthesizing the two single strand oligonucleotides of the duplex by conventional solid phase oligonucleotide synthesis. After purification, the two oligonucleotides are annealed into the duplex.

In vivo JetPEI® is a cationic polymer delivery system that binds the negatively charged siRNA molecules to the cationic polyamine polymer. Its use has been reported in xenograft models using MCF-7 (breast), MDA-MB-231 (breast) and A549 (lung) cell lines both ip and intratumoral. This delivery system is currently used in seven human clinical trials (Table 10). The formulated siRNAs are reported to be very stable.

TABLE 10
Clinical trial use of in vivo-jetPEI ®

| Organization | Type of study | Phase |
|---|---|---|
| Cancer Targeting Systems | Imaging and cancer | Pre-clinical |
| | therapy | |
| Benitec | Lung metastases | Pre-clinical |
| Avena | Blood-brain barrier | Pre-clinical |
| BiOncoTech | Melanoma immunotherapy | Phase 1 |
| Ottawa Hospital Research | Acute myocardial infarction | Phase 1 |
| Institute | gene therapy | |
| CHU-Toulouse, Rangueil | Pancreatic cancer gene | Phase 2 |
| Hospital | therapy | |
| BioCancell | Bladder cancer gene | Phase 3 |
| | therapy | |

Note that in the specification and claims, “about” or “approximately” means within twenty percent (20%) of the numerical amount cited. Although the invention has been described in detail with particular reference to these embodiments, other embodiments can achieve the same results. For example, antisense oligonucleotides that are complimentary to the target mRNA can inhibit expression of the protein of interest even though the antisense oligonucleotide is not provided as a dsRNA and may not bind to RISC/AGO complex. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. An isolated cell comprising a double stranded RNAi agent combination of (i) a first double-stranded ribonucleic acid (dsRNA) for inhibiting the expression of a CD320 gene wherein the first dsRNA comprises a sense strand and an antisense strand forming a duplex, and (ii) a second dsRNA for inhibiting the expression of a LRP2 gene wherein the second dsRNA comprises a sense strand and an antisense strand forming a duplex, and wherein the sense strand of the first dsRNA is at least substantially complementary to the antisense strand of the first dsRNA and the sense strand of the second dsRNA is at least substantially complementary to the antisense strand of the second dsRNA wherein the antisense strand of (i) the first dsRNA is selected from SEQ ID NO:1-93 and the antisense strand of (ii) the second dsRNA is selected from SEQ ID NO: 187-560 wherein * is a phosphorothioate linkage.

2. A pharmaceutical composition for inhibiting expression of a CD320 gene and a LRP2 gene, the pharmaceutical composition comprising a combination of (i) a first double-stranded ribonucleic acid (dsRNA) for inhibiting the expression of a CD320 gene wherein the first dsRNA comprises a sense strand and an antisense strand forming a duplex, and (ii) a second dsRNA for inhibiting the expression of a LRP2 gene wherein the second dsRNA comprises a sense strand and an antisense strand forming a duplex, and wherein the sense strand of the first dsRNA is at least substantially complementary to the antisense strand of the first dsRNA and the sense strand of the second dsRNA is at least substantially complementary to the antisense strand of the second dsRNA wherein the antisense strand of (i) the first dsRNA is selected from SEQ ID NO:1-93 and the antisense strand of (ii) the second dsRNA is selected from SEQ ID NO: 187-560 wherein * is a phosphorothioate linkage; and an excipient.

3. A method for inhibiting proliferation of a cancer cell (CC) comprising contacting of the CC with an inhibitor of CD320 expression and an inhibitor of LRP2 expression in an amount effective to inhibit proliferation of the CC, wherein the inhibitor of CD320 expression is a first double-stranded ribonucleic acid (dsRNA) comprising a sense strand and an antisense strand forming a duplex, and the inhibitor of LRP2 expression is a second dsRNA comprising a sense strand and an antisense strand forming a duplex, wherein the sense strand of the first dsRNA is at least substantially complementary to the antisense strand of the first dsRNA and the sense strand of the second dsRNA is at least substantially complementary to the antisense strand of the second dsRNA wherein the CC is from a skin cancer and wherein the antisense strand of (i) the first dsRNA is selected from SEQ ID NO:1-93 and the antisense strand of (ii) the second dsRNA is selected from SEQ ID NO: 187-560 wherein * is a phosphorothioate linkage.

4. The method of claim 3 wherein the contacting of the CC with an inhibitor of CD320 expression and an inhibitor of LRP2 expression is topical.

5. A method for treating skin cancer in a subject comprising administering to a subject an inhibitor of CD320 expression and an inhibitor of LRP2 expression in an amount effective to inhibit proliferation or kill cancer cells (CC) of the skin cancer wherein the CC is from the skin cancer and wherein the inhibitor is a combination of (i) a first double-stranded ribonucleic acid (dsRNA) for inhibiting the expression of a CD320 gene wherein the first dsRNA comprises a sense strand and an antisense strand forming a duplex, and (ii) a second dsRNA for inhibiting the expression of a LRP2 gene wherein the second dsRNA comprises a sense strand and an antisense strand forming a duplex, and wherein the sense strand of the first dsRNA is at least substantially complementary to the antisense strand of the first dsRNA and the sense strand of the second dsRNA is at least substantially complementary to the antisense strand of the second dsRNA wherein the antisense strand of (i) the first dsRNA is selected from SEQ ID NO:1-93 and the antisense strand of (ii) the second dsRNA is selected from SEQ ID NO: 187-560 wherein * is a phosphorothioate linkage.

6. The method of claim 5 wherein the step of administering is topical administration to the skin cancer of the subject being treated.

7. A method for treating cancer in a subject who has recurring or relapsed cancer comprising administering to a subject an inhibitor of CD320 expression and an inhibitor of LRP2 expression in an amount effective to inhibit proliferation or kill cancer cells (CC) of the cancer wherein the CC is from a skin cancer and wherein the inhibitor is a combination of (i) a first double-stranded ribonucleic acid (dsRNA) for inhibiting the expression of a CD320 gene wherein the first dsRNA comprises a sense strand and an antisense strand forming a duplex, and (ii) a second dsRNA for inhibiting the expression of a LRP2 gene wherein the second dsRNA comprises a sense strand and an antisense strand forming a duplex, and wherein the sense strand of the first dsRNA is at least substantially complementary to the antisense strand of the first dsRNA and the sense strand of the second dsRNA is at least substantially complementary to the antisense strand of the second dsRNA wherein the antisense strand of (i) the first dsRNA is selected from SEQ ID NO:1-93 and the antisense strand of (ii) the second dsRNA is selected from SEQ ID NO: 187-560 wherein * is a phosphorothioate linkage.

8. The method of claim 7 wherein the step of administering is topical administration to the skin cancer of the subject being treated.
